(19) United States
(12) Patent Application Publication
(10) Pub. No.: US 2025/0312355 A1
(43) Pub. Date: Oct. 9, 2025

(54) DHCR24 INHIBITORY COMPOUNDS

(71) Applicant: ACADEMISCH ZIEKENHUIS LEIDEN (H.O.D.N. LUMC), Leiden (NL)

(72) Inventors: Franz BRACHER, Leiden (NL); Martin Andreas GIERA, Leiden (NL); Christoph Anton MÜLLER, Munich (DE); Patrick Cornelis Nicolaas RENSEN, Leiden (NL)

(21) Appl. No.: 18/844,633

(22) PCT Filed: Mar. 7, 2023
(86) PCT No.: PCT/NL2023/050109

§ 371 Date: Sep. 6, 2024

(30) Foreign Application Priority Data
Mar. 7, 2022 (NL) 2031175

(51) Int. Cl. A61K 31/57; A61K 31/575; A61K 31/58; A61P 1/16; C07J 7/00; C07J 9/00; C07J 31/00; C07J 41/00; C07J 43/00

(52) U.S. Cl. CPC A61K 31/57; A61K 31/575; A61K 31/58; A61P 1/16; C07J 7/0005; C07J 9/00; C07J 31/006; C07J 41/005; C07J 41/0088; C07J 43/003

(57) ABSTRACT

The present disclosure relates to compounds suitable for the inhibition of 424-dehydrocholesterol reductase (DHCR24), particularly the selective inhibition of DHCR24. These compounds are for use as therapeutic agents, in particular, agents for use in the treatment and/or prevention of a DHCR24-mediated disorder, such as non-alcoholic steatohepatitis (NASH), atherosclerotic cardiovascular disease (asCVD) or multiple sclerosis (MS).

### FIELD OF THE INVENTION

The invention relates to compounds suitable for the inhibition of Δ24-dehydrocholesterol reductase (DHCR24), particularly the selective inhibition of DHCR24. These compounds are for use as therapeutic agents, in particular, therapeutic agents for use in the treatment and/or prevention of a DHCR24-mediated disorder, such as non-alcoholic steatohepatitis (NASH), atherosclerotic cardiovascular disease (asCVD) or multiple sclerosis (MS).

### BACKGROUND

Cholesterol is known to be an important storage lipid and cell-building material. The understanding of cholesterol biosynthesis and the biological role of its biosynthetic precursors has significantly evolved over the last decade. Cholesterol biosynthesis has been linked to a variety of different diseases and research has focused on the biological functions of key intermediates and enzymes involved in cholesterol biosynthesis.

Cholesterol biosynthesis is divided into the pre-mevalonate pathway and the post-squalene pathway, the latter is also known as distal cholesterol biosynthesis. De novo, cholesterol biosynthesis is accomplished by eleven enzymes within the mevalonate pathway (starting from acetyl coenzyme A) and nine enzymes take part in distal cholesterol biosynthesis. The latter is further divided into the Bloch and Kandutsch-Russell pathways (see FIG. 1).

The Bloch pathway comprises the Δ24-unsaturated intermediates and is interconnected with the Kandutsch-Russell branch by the actions of the enzyme DHCR24. DHCR24 is a membrane-bound enzyme that catalyses the anaerobic reduction of the Δ24-double bond in the side chain of precursor sterols (see FIGS. 1 and 2). It has been shown that both pathways are not strictly separated, but rather a tissue and cell-type specific interaction of both pathways with a preference for the Kandutsch-Russel pathway is observed. The predominant substrates of DHCR24 are lanosterol (4,4,14-trimethylcholesta-8,24-dien-3β-ol, see (1) in FIG. 1) and zymosterol (cholesta-8,24-dien-3β-ol, see (11) in FIG. 1), as well as cholesta-7,24-dien-3β-ol (see (12) in FIG. 1).

Reduction of the Δ24 double bond can take place in the final step of the Bloch pathway converting desmosterol (cholesta-5,24-dien-3β-ol, see (14) in FIGS. 1 and 2) into cholesterol (see (8) in FIGS. 1 and 2). DHCR24 needs no cofactors other than NADPH. The reduction of the Δ24 double bond proceeds in two steps through an initial introduction of a proton at C24 generating a cationic high energy intermediate (HEI) at C25, followed by nucleophilic addition of hydride from NADPH (see FIG. 2). Dysfunction or inhibition of DHCR24 causes mammalian cholesterol biosynthesis to proceed via the Bloch pathway, ultimately leading to the accumulation of desmosterol.

Desmosterolosis (MIM 602398) is a rare genetic disorder affecting the DHCR24 gene. Desmosterolosis is a very rare disease with only a few clinically described cases. Desmosterolosis is accompanied by severe abnormalities, such as microcephaly with agenesis of the corpus callosum, convulsions, nystagmus, strabismus, and micrognathia. It has been found that a mild accumulation of desmosterol has no influence on vitality, especially in combination with a cholesterol-rich diet, as exemplified by heterozygous carriers of a DHCR24 mutation. Hence, a moderate in vivo accumulation of desmosterol by inhibiting DHCR24 proves non-toxic. Carriers of a DHCR24 mutation on a single allele have been shown to possess normal cholesterol levels with only a 1.5-fold increased plasma concentration of desmosterol.

Biologically, the role of DHCR24 is diverse and the inhibition of DHCR24 is a promising drug target for the treatment of a variety of diseases. There is therefore a need for selective, potent and non-toxic inhibitors of DHCR24, which may be useful in many therapeutic areas.

### SUMMARY OF THE INVENTION

In a first aspect, the invention provides a compound of formula (10) or a salt, solvate, hydrate or prodrug thereof. The compound of formula (10) is:

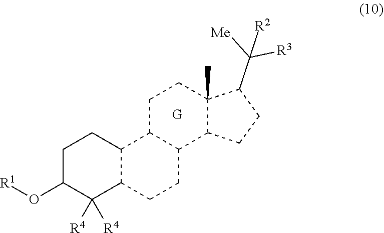

wherein:
G is a fused ring system selected from:

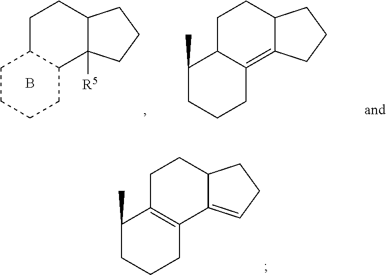

B is selected from:

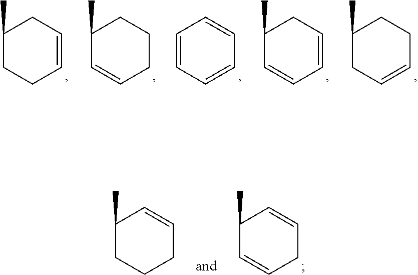

R¹ is selected from hydrogen, —C(═O)R⁶ and C₁₋₆ alkyl;
R² is selected from hydrogen and C₁₋₆ alkyl;
R³ is selected from a group represented by formula (2), —[C(R⁷)₂]ₙ—X, —CH═CR⁶R⁷, —CR⁷═N—N(R⁶)₂, 5- to 10-membered heteroaryl and 5- to 10-membered heterocycloalkyl, wherein the 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy;

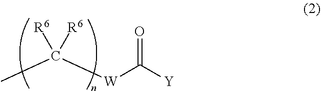

n is an integer from 1 to 6;
W is selected from O and NR⁶;
Y is selected from hydrogen and C₂ alkenyl, wherein the C₂ alkenyl is optionally substituted with one or more halo;
X is selected from halo, —OH, —SH, —O—Z, —S—Z, —S—S—Z, 5- to 10-membered heteroaryl and 5- to 10-membered heterocycloalkyl, wherein the 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy;
each R⁴ is independently selected from hydrogen and C₁₋₆ alkyl;
R⁵ is selected from hydrogen and C₁₋₆ alkyl;
each R⁶ is independently selected from hydrogen and C₁₋₆ alkyl;
each R⁷ is independently selected from H, C₁₋₆ alkyl and C₂₋₆ alkenyl, wherein the C₁₋₆ alkyl and C₂₋₆ alkenyl are optionally substituted with one or more halo;
Z is selected from C₆₋₁₀ aryl, 5- to 10-membered heteroaryl and 5- to 10-membered heterocycloalkyl, wherein the C₆₋₁₀ aryl, 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy.

The compound of formula (10) is not one of:

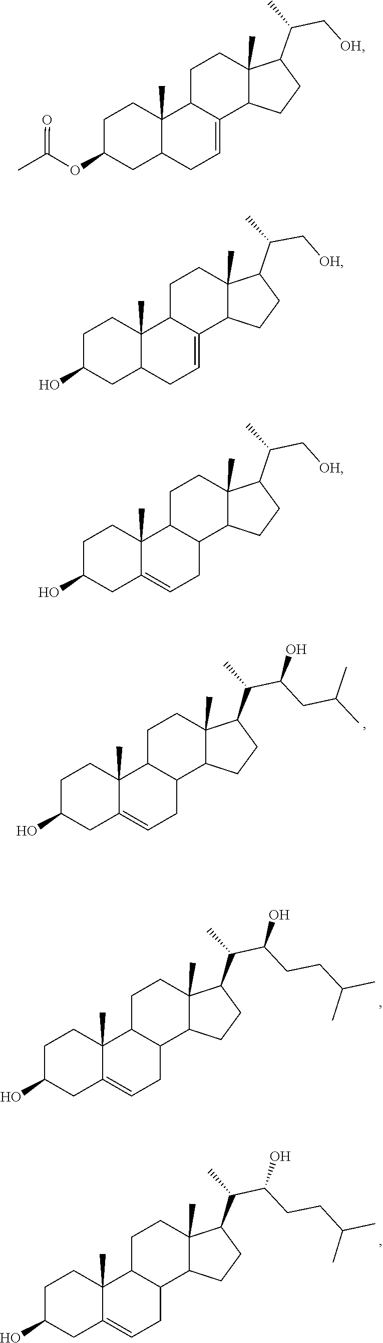 [SMILES: CC(=O)O[C@H]1CC[C@@]2(C)C(CC=C3C2CC[C@@]2(C)C3CCC2[C@H](C)CO)C1 ; C[C@H](CO)C1CCC2C3=CCC4C[C@@H](O)CC[C@]4(C)C3CC[C@@]21C ; C[C@H](CO)C1CCC2C3CC=C4C[C@@H](O)CC[C@]4(C)C3CC[C@@]21C ; CC(C)C[C@H](O)[C@@H](C)[C@H]1CCC2C3CC=C4C[C@@H](O)CC[C@]4(C)C3CC[C@@]21C ; CC(C)CC[C@H](O)[C@@H](C)[C@H]1CCC2C3CC=C4C[C@@H](O)CC[C@]4(C)C3CC[C@@]21C ; CC(C)CC[C@@H](O)[C@@H](C)[C@H]1CCC2C3CC=C4C[C@@H](O)CC[C@]4(C)C3CC[C@@]21C]

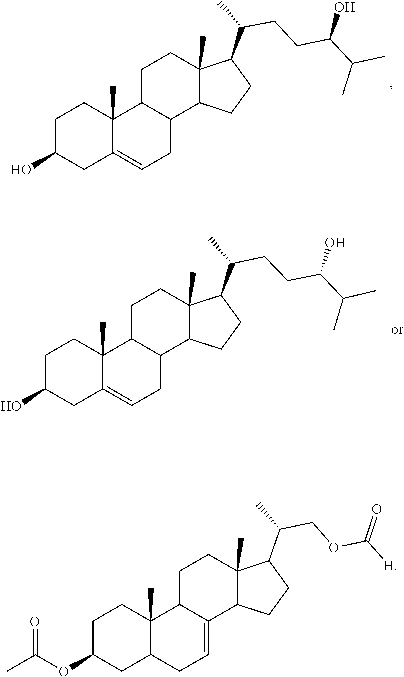

The dashed lines in formula (10) represent the position of the fused ring system represented by G. Similarly, the dashed lines in one of the structures for G represents the position of the group represented by B.

The inventors have unexpectedly identified a class of compounds that inhibit DHCR24. The compounds are selective, potent and non-toxic inhibitors of DHCR24. The selectivity of the compounds toward inhibiting DHCR24 is important for their medicinal or clinical use in the treatment or prevention of a DHCR24-mediated disorder, given the diverse biological role of DHCR24. The inhibition of DHCR24 by the compounds of the invention increases endogenous desmosterol levels.

In comparison to other inhibitors of DHCR24, the compounds identified by the invention have properties that are advantageous when formulating a drug product for clinical use (e.g. Lipinski's rules may apply). Thus, the compounds have increased water solubility and better stability. The compounds are also expected to be suitable for direct oral administration.

A further aspect of the invention provides a compound of formula (10) or a pharmaceutically acceptable salt, solvate, hydrate or prodrug thereof, for use in the treatment or prevention of a DHCR24-mediated disorder. This aspect of the invention also provides a method of treating or preventing a DHCR24-mediated disorder. The method comprises administering to a subject a compound of formula (10) or a pharmaceutically acceptable salt, solvate, hydrate or prodrug thereof.

In a second aspect, the invention provides a compound of formula (1) or a pharmaceutically acceptable salt, solvate, hydrate or prodrug thereof, for use in the treatment or prevention of a DHCR24-mediated disorder.

The second aspect of the invention also provides a method of treating or preventing a DHCR24-mediated disorder. The method comprises administering to a subject a compound of formula (1) or a pharmaceutically acceptable salt, solvate, hydrate or prodrug thereof.

In the second aspect of the invention, the compound of formula (1) is:

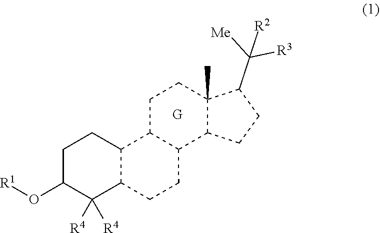

wherein:
G is a fused ring system selected from:

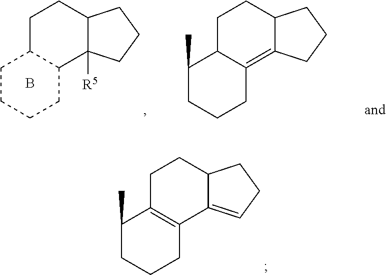

B is selected from:

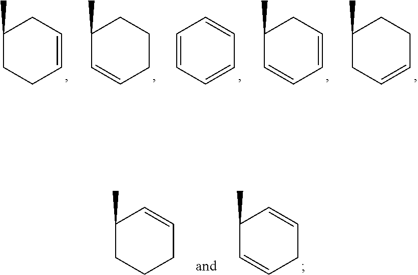

R¹ is selected from hydrogen, —C(═O)R⁶ and C₁₋₆ alkyl;
R² is selected from hydrogen and C₁₋₆ alkyl;
R³ is selected from a group represented by formula (2), —[C(R⁷)₂]ₙ—X, —C(═O)NHOR⁸, —C(═O)OR⁸, —CR⁷═N—N(R⁶)₂, —CH═CR⁶R⁷, C₆₋₁₀ aryl, 5- to 10-membered heteroaryl and 5- to 10-membered heterocycloalkyl, wherein the C₆₋₁₀ aryl, 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy;

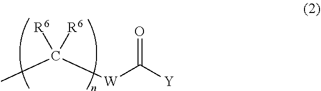

n is an integer from 1 to 6;
W is selected from O, S and NR⁶;
Y is selected from hydrogen, C₁₋₆ alkyl, C₂₋₆ alkenyl, amino-C₁₋₆ alkyl, (mono-C₁₋₆ alkylamino) C₁₋₆ alkyl and (di-C₁₋₆ alkylamino)C₁₋₆ alkyl, wherein the C₁₋₆ alkyl and C₂₋₆ alkenyl are optionally substituted with one or more halo;
X is selected from halo, —OH, —SH, —N(R⁶)₂, —O—Z, —S—Z, —S—S—Z, —C(═O)R⁷, C₆₋₁₀ aryl, 5- to 10-membered heteroaryl and 5- to 10-membered heterocycloalkyl, wherein the C₆₋₁₀ aryl, 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy;
each R⁴ is independently selected from hydrogen and C₁₋₆ alkyl;
R⁵ is selected from hydrogen and C₁₋₆ alkyl;
each R⁶ is independently selected from hydrogen and C₁₋₆ alkyl;
each R⁷ is independently selected from H, C₁₋₆ alkyl and C₂₋₆ alkenyl, wherein the C₁₋₆ alkyl and C₂₋₆ alkenyl are optionally substituted with one or more halo;
R⁸ is selected from C₁₋₆ alkyl and C₂₋₆ alkenyl; and
Z is selected from C₆₋₁₀ aryl, 5- to 10-membered heteroaryl and 5- to 10-membered heterocycloalkyl, wherein the C₆₋₁₀ aryl, 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy.

The dashed lines in formula (1) represent the position of the fused ring system represented by G. Similarly, the dashed lines in one of the structures for G represents the position of the group represented by B.

The experimental data presented herein, particularly the in vivo data, provide compelling evidence that the compounds can be used clinically in the treatment or prevention of a DHCR24-mediated disorder. The compounds can produce an effect within the brain (e.g. it is not prevented by the blood-brain barrier).

Another aspect of the invention is the provision of a pharmaceutical composition. The pharmaceutical composition comprises a compound of formula (1) or (10) as defined herein, or a pharmaceutically acceptable salt, solvate, hydrate or prodrug thereof, and a pharmaceutically acceptable excipient.

In another aspect, the invention provides a compound of formula (1) or formula (10) as defined herein, or a pharmaceutically acceptable salt, solvate, hydrate or prodrug thereof, or a pharmaceutical composition as defined herein, for use in therapy and/or for use as a medicament.

In another aspect, the present invention provides the use of the compound of formula (1) or formula (10), or a pharmaceutically acceptable salt, solvate, hydrate or prodrug thereof, in the manufacture of a medicament for the treatment or prevention of a DHCR24-mediated disorder.

A further aspect of the invention provides a method of inhibiting the activity of DHCR24 in vivo or in vitro. The in vivo or the in vitro method comprises contacting a cell with the compound of formula (1) or formula (10), or a pharmaceutically acceptable salt, solvate, hydrate or prodrug thereof, or a pharmaceutical composition as defined herein. Alternatively, the in vivo method may comprise administering to a subject a compound of formula (1) or formula (10), or a pharmaceutically acceptable salt, solvate, hydrate or prodrug thereof, or a pharmaceutical composition as defined herein.

In another aspect, the present invention provides a combination comprising a compound of formula (1) or formula (10), or a pharmaceutically acceptable salt, solvate, hydrate or prodrug thereof, with one or more additional therapeutic agents.

Preferred, suitable, and optional features of any one particular aspect of the invention are also preferred, suitable, and optional features of any other aspect.

### BRIEF DESCRIPTION OF THE DRAWINGS

The invention is further described hereinafter with reference to the accompanying drawings.

FIG. 1 is a scheme showing the enzymatic steps of distal cholesterol biosynthesis. Involved enzymes: (A) sterol C24-reductase (Δ24-dehydrocholesterol reductase (DHCR24)), (B) sterol C14-demethylase (CYP51), (C) sterol C14-reductase (Δ14-dihydrocholesterol reductase (DHCR14)), (D) sterol C4-methyl oxidase, (E) sterol C3-dehydrogenase, (F) sterol C3-keto reductase, (G) sterol C8-isomerase (emapomil binding protein (EBP)), (H) sterol C5-desaturase (SC5D)), (I) sterol C7-reductase (Δ7-dehydrocholesterol reductase (DHCR7)). Intermediates: (1) 4,4,14-trimethylcholesta-8,24-dien-3β-ol (lanosterol), (2) 4,4,14-trimethylcholesta-8-en-3β-ol (dihydrolanosterol), (3) 4,4-dimethylcholesta-8,14-dien-3β-ol, (4) 4,4-dimethylcholest-8-en-3β-ol, (5) cholest-8-en-3β-ol (zymostenol), (6) cholest-7-en-3β-ol (lathosterol), (7) cholesta-5,7-dien-3β-ol (7-dehydrocholesterol), (8) cholest-5-en-3β-ol (cholesterol), (9) 4,4-dimethylcholesta-8,14,24-trien-3β-ol (follicular fluid meiosis activating sterol (FF-MAS)), (10) 4,4-dimethylcholesta-8,24-dien-3β-ol (testis meiosis activating sterol (T-MAS)), (11) cholesta-8,24-dien-3β-ol (zymosterol), (12) cholesta-7,24-dien-3β-ol, (13) cholesta-5,7,24-trien-3β-ol (7-dehydrocholesterol), (14) cholesta-5,24-dien-3β-ol (desmosterol).

FIG. 2 is a reaction scheme showing the postulated mechanism of the DHCR24 enzyme reaction.

FIG. 3 is a series of histograms showing the results of various in vivo experiments using Example 11 (SH42).

FIGS. 4 and 5 show the results of in vivo experiments in which the levels of desmosterol were measured in the brains of mice after treatment with Example 11 or Example 33.

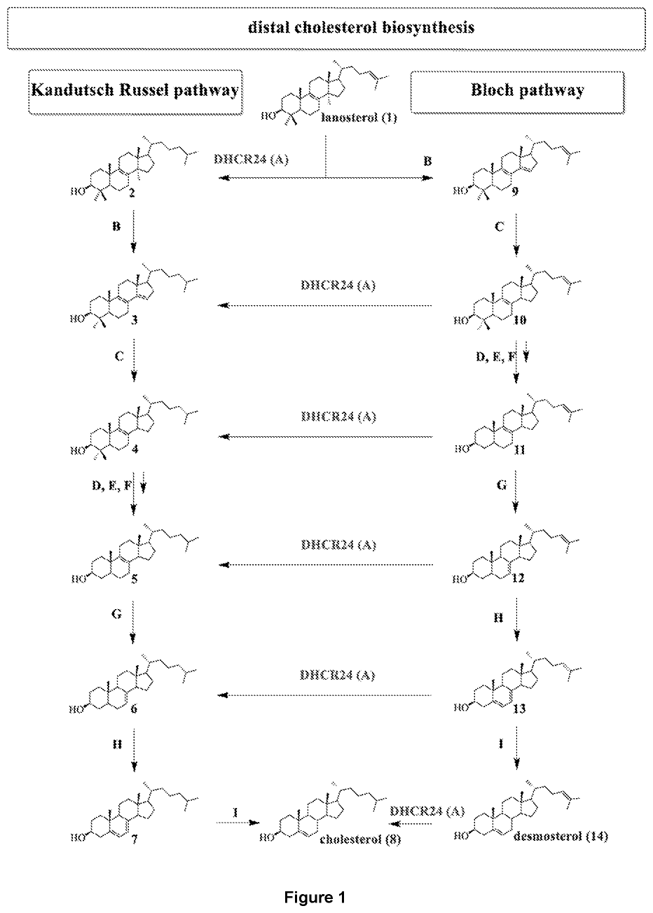

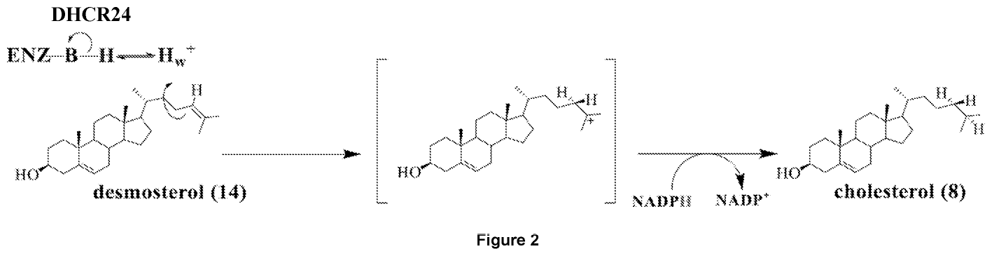

### DETAILED DESCRIPTION

### Definitions

The compounds and intermediates described herein may be named according to either the IUPAC (International Union for Pure and Applied Chemistry) or CAS (Chemical Abstracts Service) nomenclature systems. It should be understood that unless expressly stated to the contrary, the terms “compounds of formula (1)” and “compounds of formula (10)” refer to and include any and all compounds described by and/or with reference to formula (1) or formula (10), respectively. It should also be understood that these terms encompass all stereoisomers, i.e. cis and trans isomers, as well as optical isomers, i.e. R and S enantiomers and diastereomers, of such compounds and all salts thereof, in substantially pure form and/or any mixtures of the foregoing in any ratio. This understanding extends to pharmaceutical compositions and methods of treatment that employ or comprise one or more compounds of formula (1) or formula (10), either by themselves or in combination with additional agents.

The various hydrocarbon-containing moieties provided herein may be described using a prefix designating the minimum and maximum number of carbon atoms in the moiety, e.g. “C_{a-b}” or “Cₐ-C_{b}”. For example, C_{a-b} alkyl indicates an alkyl moiety having the integer “a” to the integer “b” number of carbon atoms, inclusive. Certain moieties may also be described according to the minimum and maximum number of members with or without specific reference to a particular atom or overall structure. For example, the terms “a to b membered ring” or “having between a to b members” refer to a moiety having the integer “a” to the integer “b” number of atoms, inclusive.

The term “about” when used herein in conjunction with a measurable value such as, for example, an amount or a period of time and the like, is meant to encompass reasonable variations of the value, for instance, to allow for experimental error in the measurement of said value.

As used herein by itself or in conjunction with another term or terms, the term “alkyl” or “alkyl group” refer to a branched or unbranched saturated hydrocarbon chain. Unless specified otherwise, alkyl groups typically contain 1-6 carbon atoms, such as 1-4 carbon atoms or 1-3 carbon atoms, and can be substituted or unsubstituted. The alkyl group is unsubstituted, unless the context indicates otherwise. Representative examples include, but are not limited to, methyl, ethyl, n-propyl, i-propyl, n-butyl, i-butyl, s-butyl, t-butyl, n-pentyl, n-hexyl, isopropyl, tert-butyl, isobutyl, etc.

As used herein by itself or in conjunction with another term or terms, the term “alkenyl” or “alkenyl group” refers to a branched or unbranched hydrocarbon chain containing at least one double bond. Unless specified otherwise, alkenyl groups typically contain 2-6 carbon atoms, such as 2-4 carbon atoms, and can be substituted or unsubstituted. The alkenyl group is unsubstituted, unless the context indicates otherwise. Representative examples include, but are not limited to, ethenyl, 3-buten-1-yl, 2-ethenylbutyl, and 3-hexen-1-yl.

As used herein by itself or in conjunction with another term or terms, the term “amino-alkyl” or “amino-alkyl group” refers to an alkyl group in which one hydrogen atom, preferably is replaced by a primary amino group (—NH₂). The term “amino-C₁₋₆ alkyl” and “amino-C₁₋₆ alkyl group” refers to an amino-alkyl group in which the alkyl group contains 1-6 carbon atoms. Representative examples include, but are not limited to, —CH₂NH₂, CH₂CH₂NH₂, and —CH(CH₃)NH₂. Amino-alkyl groups can be substituted or unsubstituted. The amino-alkyl group is unsubstituted, unless the context indicates otherwise.

As used herein by itself or in conjunction with another term or terms, the term “(mono-alkylamino)-alkyl” or “(mono-alkylamino)-alkyl group” refers to an alkyl group in which one hydrogen atom, preferably is replaced by an amino group (—NHR′), in which the amino group has a single alkyl substituent (e.g. R′). The term “mono-C₁₋₆ alkylamino” in the expression “(mono-C₁₋₆ alkylamino) C₁₋₆ alkyl” refers to the single alkyl group on the amino group, which contains 1-6 carbon atoms. Representative examples include, but are not limited to, —CH₂NH(CH₃), —CH₂NH(CH₂CH₃), and —CH₂CH₂NH(CH₃). The (mono-alkylamino)-alkyl groups can be substituted or unsubstituted. The (mono-alkylamino)-alkyl group is unsubstituted, unless the context indicates otherwise.

As used herein by itself or in conjunction with another term or terms, the term “(di-alkylamino)-alkyl” or “(di-alkylamino)-alkyl group” refers to an alkyl group in which one hydrogen atom, preferably is replaced by an amino group (—NR′₂), in which the amino group has two alkyl substituents (e.g. each represented by R′). The term “di-C₁₋₆ alkylamino” in the expression “(di-C₁₋₆ alkylamino) C₁₋₆ alkyl” refers to the two alkyl groups on the amino group, where each alkyl group independently contains 1-6 carbon atoms. Representative examples include, but are not limited to, —CH₂N(CH₃)₂, —CH₂N(CH₂CH₃)(CH₃), and —CH₂CH₂N(CH₃)₂. The (di-alkylamino)-alkyl groups can be substituted or unsubstituted. The (di-alkylamino)-alkyl group is unsubstituted, unless the context indicates otherwise.

As used herein by itself or in conjunction with another term or terms, the term “alkoxy” and “alkoxy group” refers to an alkyl-oxy group, i.e. an alkyl group in which one hydrogen atom is replaced by an oxy group (—O—). Representative examples include, but are not limited to, OCH₃, OCH₂CH₃ and OCH(CH₃)₂. Alkoxy groups can be substituted or unsubstituted. The alkoxy group is unsubstituted, unless the context indicates otherwise.

As used herein by itself or in conjunction with another term or terms, the term “aromatic” refers to monocyclic and polycyclic ring systems containing 4n+2 pi electrons, where n is an integer. Aromatic should be understood as referring to and including ring systems that contain only carbon atoms (i.e. “aryl”) as well as ring systems that contain at least one heteroatom selected from N, O or S (i.e. “heteroaromatic” or “heteroaryl”). An aromatic ring system can be substituted or unsubstituted.

As used herein by itself or in conjunction with another term or terms, the term “non-aromatic” refers to a monocyclic or polycyclic ring system that is saturated or has at least one double bond that is not part of an extended conjugated pi system. As used herein, non-aromatic refers to and includes ring systems that contain only carbon atoms as well as ring systems that contain at least one heteroatom selected from N, O or S. A non-aromatic ring system can be substituted or unsubstituted.

As used herein by itself or in conjunction with another term or terms, the term “aryl” or “aryl group” refers to phenyl and 6-10 membered bicyclic hydrocarbon ring systems, including fused ring systems, in which at least one of the rings is aromatic. Aryl groups can be substituted or unsubstituted. Unless specified otherwise, an aryl group may contain 6 ring atoms (i.e., phenyl) or a ring system containing 6 to 10 atoms, such as 9 or 10 ring atoms. Representative examples include, but are not limited to, naphthyl, indanyl and 1,2,3,4-tetrahydronaphthalenyl. It is preferable that the aryl group is phenyl or naphthyl, more preferably phenyl. Aryl groups can be substituted or unsubstituted. The aryl group is unsubstituted, unless the context indicates otherwise.

As used herein by itself or in conjunction with another term or terms, the terms “halo”, “halo group”, “halogen” and “halogen group” include fluoro (—F), chloro (—Cl), bromo (—Br) or iodo (—I) atoms and substituents.

As used herein by itself in conjunction with another term or terms, the term “heteroaryl” or “heteroaryl group” refers to:
(a) 5- and 6-membered monocyclic aromatic rings, which contain, in addition to carbon atom(s), at least one heteroatom, such as nitrogen, oxygen or sulfur; and
(b) 7- to 10-membered bicyclic rings, which contain, in addition to carbon atom(s), at least one heteroatom, such as nitrogen, oxygen or sulfur;
   and in which at least one of the rings is aromatic. In some instances, a heteroaryl group can contain two or more heteroatoms, which may be the same or different. Heteroaryl groups can be substituted or unsubstituted, and may be fused. In some instances, a heteroaryl group may contain 5, 6, or 8 to 10 ring atoms. In other instances, a heteroaryl group may contain 5 to 10 ring atoms, such as 5, 6, 9, or 10 ring atoms. Representative examples include, but are not limited to, 2,3-dihydrobenzofuranyl, 1,2-dihydroquinolinyl, 3,4-dihydroisoquinolinyl, 1,2,3,4-tetrahydroisoquinolinyl, 1,2,3,4-tetrahydroquinolinyl, benzoxazinyl, benzothiazinyl, chromanyl, furanyl (e.g. 2-furanyl, 3-furanyl), imidazolyl, isoxazolyl, isothiazolyl, oxadiazolyl, oxazolyl, pyridinyl (e.g. 2-, 3-, or 4-pyridinyl), pyrimidinyl (e.g. 2-, 4-, or 5-pyrimidinyl), pyrazolyl, pyrrolyl (e.g. 2- or 3-pyrrolyl), pyrazinyl, pyridazinyl (e.g. 3- or 4-pyridazinyl, 2-pyrazinyl), thienyl (e.g. 2-thienyl, 3-thienyl), tetrazolyl, thiazolyl, thiadiazolyl, triazinyl, triazolyl, pyridin-2-yl, pyridin-4-yl, pyrimidin-2-yl, pyridazin-4-yl, pyrazin-2-yl, naphthyridinyl, pteridinyl, phthalazinyl, purinyl, benzimidazolyl, benzofuranyl, benzofurazanyl, 2H-1-benzopyranyl, benzothiadiazine, benzothiazinyl, benzothiazolyl, benzothiophenyl, benzoxazolyl, cinnolinyl, furopyridinyl, indolinyl, indolizinyl, indolyl, or 2-, 3-, 4-, 5-, 6-, or 7-indolyl, 3H-indolyl, quinazolinyl, quinoxalinyl, isoindolyl and isoquinolinyl. It is preferred that a heteroaryl is a 5- or 6-membered heteroaryl ring comprising one, two or three heteroatoms selected from N, O or S. The heteroaryl group is unsubstituted, unless the context indicates otherwise.

As used herein by themselves or in conjunction with another term or terms, the term “heterocycloalkyl” or “heterocycloalkyl group” refer to a 3- to 10-membered monocyclic or bicyclic, non-aromatic ring system, which contains, in addition to carbon atom(s), at least one heteroatom, such as nitrogen, oxygen, sulfur or phosphorus. Heterocycloalkyl groups may be fully saturated or contain unsaturated portions and may be bridged, spiro, and/or fused ring systems. In some instances, a heterocycloalkyl group may contain at least two or heteroatoms, which may be the same or different. Heterocycloalkyl groups can be substituted or unsubstituted. In some instances, a heterocycloalkyl group may contain from 3 to 10 ring atoms or from 3 to 7 ring atoms or from 5 to 7 ring atoms, such as 5 ring atoms, 6 ring atoms, or 7 ring atoms. Representative examples include, but are not limited to, tetrahydrofuranyl, pyrrolidinyl, pyrrolinyl, imidazolidinyl, imidazolinyl, pyrazolidinyl, pyrazolinyl, piperidyl, piperazinyl, indolinyl, isoindolinyl, morpholinyl, thiomorpholinyl, homomorpholinyl, homopiperidyl, homopiperazinyl, thiomorpholinyl-5-oxide, thiomorpholinyl-S,S-dioxide, pyrrolidinyl, tetrahydropyranyl, piperidinyl, tetrahydrothienyl, homopiperidinyl, homothiomorpholinyl-S, S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, azetidinyl, azepanyl, diazepanyl (such as 1,4-diazepanyl), oxazepanyl (such as 1,4-oxazepanyl), thiazepanyl (such as 1,4-thiazepanyl), tetrahydrothienyl-5-oxide, tetrahydrothienyl-S,S-dioxide, homothiomorpholinyl-5-oxide, quinuclidinyl, 2-oxa-5-azabicyclo[2.2.1]heptanyl, 8-oxa-3-aza-bicyclo[3.2.1]octanyl, 3,8-diaza-bicyclo[3.2.1]octanyl, 2,5-diaza-bicyclo[2.2.1]heptanyl, 3,8-diaza-bicyclo[3.2.1]octanyl, 3,9-diaza-bicyclo[4.2.1]nonanyl, and 2,6-diaza-bicyclo[3.2.2]nonanyl. It is preferred that the heterocyclylalkyl group as defined herein is a monocyclic, bicyclic or spiro group comprising one, two or three heteroatoms selected from N, O or S. The heterocycloalkyl group is unsubstituted, unless the context indicates otherwise.

As used herein by itself or in conjunction with another term or terms, the term “pharmaceutically acceptable” refers to materials that are generally chemically and/or physically compatible with other ingredients (such as, for example, with reference to a formulation), and/or is generally physiologically compatible with the recipient (such as, for example, a subject) thereof.

As used herein by itself or in conjunction with another term or terms, the term “pharmaceutical composition” refers to a composition that can be used to treat a disease, condition, or disorder in a subject, including a human.

As used herein by themselves or in conjunction with another term or terms, the terms “subject(s)” and “patient(s)”, suitably refer to mammals, in particular humans.

As used herein by itself or in conjunction with another term or terms, the term “substituted” indicates that a hydrogen atom on a molecule has been replaced with a different atom or group of atoms and the atom or group of atoms replacing the hydrogen atom is a “substituent.” It should be understood that the terms “substituent”, “substituents”, “moiety”, “moieties”, “group”, or “groups” refer to substituent(s).

As used herein by themselves or in conjunction with another term or terms, the terms “therapeutic” and “therapeutically effective amount” refer to an amount a compound, composition or medicament that (a) inhibits or causes an improvement in a particular disease, condition or disorder; (b) attenuates, ameliorates or eliminates one or more symptoms of a particular disease, condition or disorder; (c) or delays the onset of one or more symptoms of a particular disease, condition or disorder described herein. It should be understood that the terms “therapeutic” and “therapeutically effective” encompass any one of the aforementioned effects (a)-(c), either alone or in combination with any of the others (a)-(c). It should be understood that in, for example, a human or other mammal, a therapeutically effective amount can be determined experimentally in a laboratory or clinical setting, or a therapeutically effective amount may be the amount required by the guidelines of the United States Food and Drug Administration (FDA) or equivalent foreign regulatory body, for the particular disease and subject being treated. It should be appreciated that determination of proper dosage forms, dosage amounts, and routes of administration is within the level of ordinary skill in the pharmaceutical and medical arts.

As used herein whether by themselves or in conjunction with another term or terms, the terms “treating”, “treated” and “treatment” refer to and include prophylactic, ameliorative, palliative, and curative uses and results. In some embodiments, the terms “treating”, “treated”, and “treatment” refer to curative uses and results as well as uses and results that diminish or reduce the severity of a particular condition, characteristic, symptom, disorder, or disease described herein. For example, treatment can include diminishment of several symptoms of a condition or disorder or complete eradication of said condition or disorder. It should be understood that the term “prophylactic” as used herein is not absolute but rather refers to uses and results where the administration of a compound or composition diminishes the likelihood or seriousness of a condition, symptom, or disease state, and/or delays the onset of a condition, symptom, or disease state for a period of time.

As used herein, a “therapeutically active agent”, whether used alone or in conjunction with another term or terms, refers to any compound, i.e. a drug, that has been found to be useful in the treatment of a disease, disorder or condition and is not described by formula (1) or formula (10). It should be understood that a therapeutically active agent may not be approved by the FDA or an equivalent foreign regulatory body.

A “therapeutically effective amount” means the amount of a compound that, when administered to a subject or patient for treating a disease, is sufficient to effect such treatment for the disease. The “therapeutically effective amount” will vary depending on the compound, the disease and its severity and the age, weight, etc., of the subject or patient to be treated.

As used herein, the term “DHCR24-mediated disorder” means any disease, disorder, or other pathological condition in which DHCR24 and/or desmosterol, preferably DCHR24, is known to play a role. In particular, the term “DHCR24-mediated disorder” includes any disease, disorder or other pathological conditions that can be treated and/or prevented by inhibition of DHCR24. Inhibition of DHCR24 increases desmosterol, which activates the liver X receptor, particularly liver X receptor alpha (LXRα). Accordingly, in some embodiments, the present disclosure relates to treating or lessening the severity of one or more diseases in which DHCR24 is known to play a role.

### DESCRIPTION OF THE INVENTION

In general, the invention relates to a compound of formula (1) or a compound of formula (10).

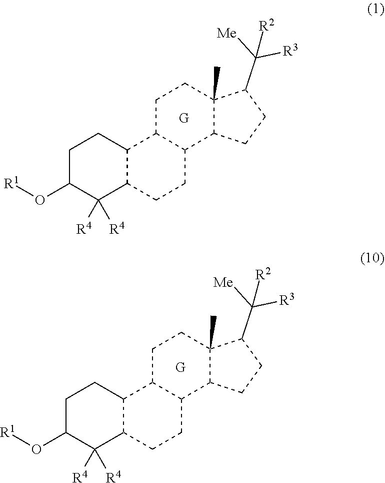

In formula (1) or formula (10), G is a fused ring system selected from:

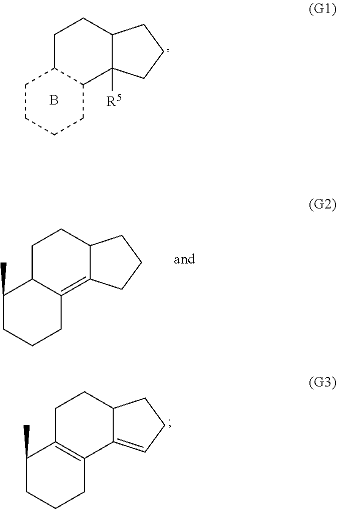

When G in formula (1) or formula (10) is a fused ring system represented by (G1), then the compound may be represented by formula (2-G1) or formula (20-G1) respectively.

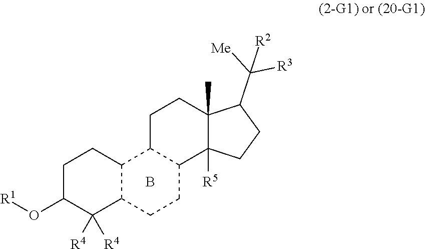

When G in formula (1) or formula (10) is a fused ring system represented by (G2), then the compound may be represented by formula (2-G2) or formula (20-G2) respectively.

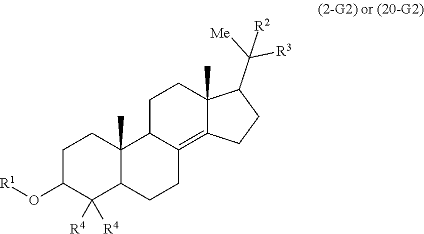

When G in formula (1) or formula (10) is a fused ring system represented by (G3), then the compound may be represented by formula (2-G3) or formula (20-G3) respectively.

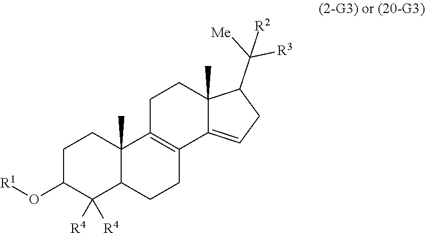

When G is a fused ring system represented by (G1), then B is selected from:

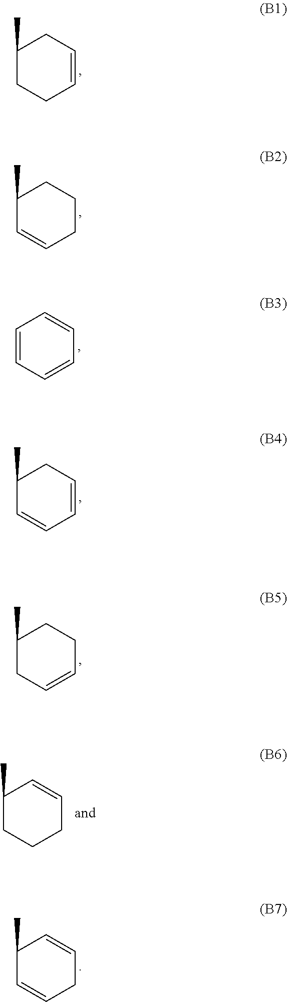

When B is represented by formula (B1) and the compound is represented by formula (2-G1) or formula (20-G1), then the compound may be represented by formula (2-B1) or formula (20-B1) respectively.

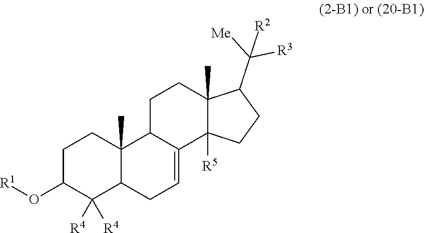

When B is represented by formula (B2) and the compound is represented by formula (2-G1) or formula (20-G1), then the compound may be represented by formula (2-B2) or formula (20-B2) respectively.

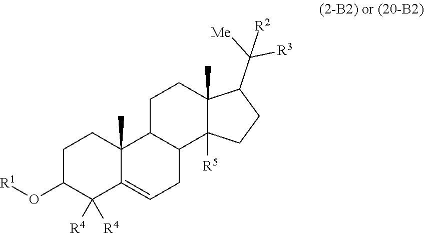

When B is represented by formula (B3) and the compound is represented by formula (2-G1) or formula (20-G1), then the compound may be represented by formula (2-B3) or formula (20-B3) respectively.

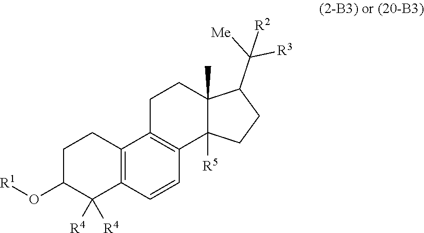

When B is represented by formula (B4) and the compound is represented by formula (2-G1) or formula (20-G1), then the compound may be represented by formula (2-B4) or formula (20-B4) respectively.

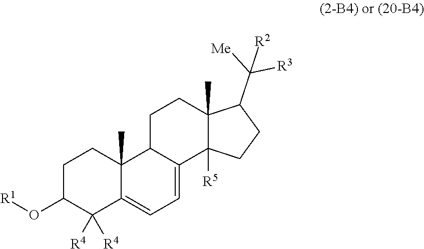

When B is represented by formula (B5) and the compound is represented by formula (2-G1) or formula (20-G1), then the compound may be represented by formula (2-B5) or formula (20-B5) respectively.

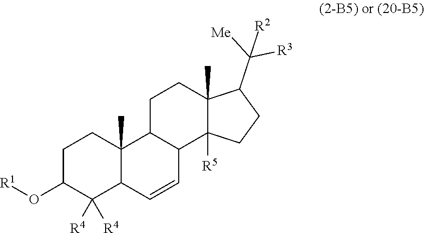

When B is represented by formula (B6) and the compound is represented by formula (2-G1) or formula (20-G1), then the compound may be represented by formula (2-B6) or formula (20-B6) respectively.

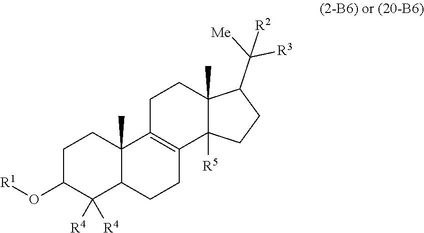

When B is represented by formula (B7) and the compound is represented by formula (2-G1) or formula (20-G1), then the compound may be represented by formula (2-B3) or formula (20-B3) respectively.

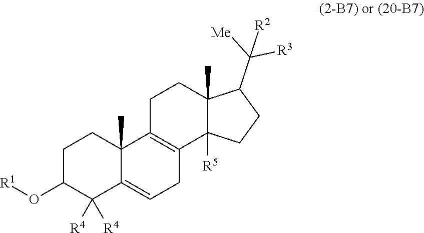

In the compound of formula (1), formula (10) or any of the formulae above, R³ may represented by formula (2).

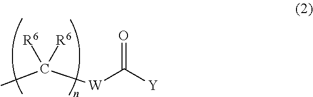

When R³ is represented by formula (2), then the compound of formula (1) or formula (10) may be represented by formula (1-R³) or formula (10-R³) respectively.

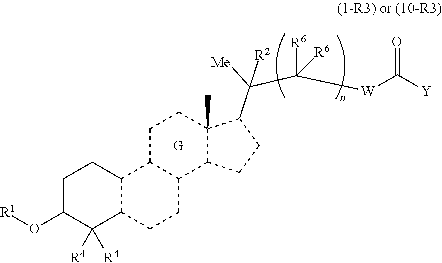

The invention will now be further described by way of the following numbered paragraphs.

In a first aspect, the invention provides a compound of formula (10) or a salt, solvate, hydrate or prodrug thereof, as defined herein above and below. The salt is preferably a pharmaceutically acceptable salt.

1. The compound of formula (10) is:

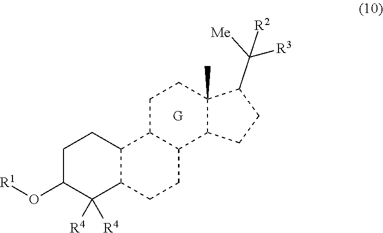

wherein:
G is a fused ring system selected from:

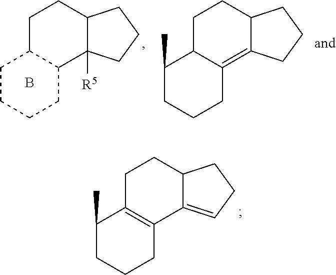

B is selected from:

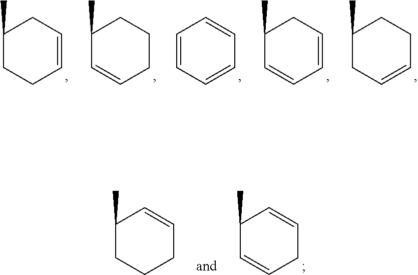

R¹ is selected from hydrogen and —C(═O)R⁶;
R² is selected from hydrogen and C₁₋₆ alkyl;
R³ is selected from a group represented by formula (2), —[C(R⁷)₂]ₙ—X, —CH═CR⁶R⁷, —CR⁷═N—N(R⁶)₂, 5- to 10-membered heteroaryl and 5- to 10-membered heterocycloalkyl, wherein the 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy;

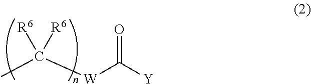

n is an integer from 1 to 6;
W is selected from O and NR⁶;
Y is selected from hydrogen and C₂ alkenyl, wherein the C₂ alkenyl is optionally substituted with one or more halo;
X is selected from halo, —OH, —SH, —O—Z, —S—Z, —S—S—Z, 5- to 10-membered heteroaryl and 5- to 10-membered heterocycloalkyl, wherein the 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy;
each R⁴ is independently selected from hydrogen and C₁₋₆ alkyl;
R⁵ is selected from hydrogen and C₁₋₆ alkyl;
each R⁶ is independently selected from hydrogen and C₁₋₆ alkyl;
each R⁷ is independently selected from H, C₁₋₆ alkyl and C₂₋₆ alkenyl, wherein the C₁₋₆ alkyl and C₂₋₆ alkenyl are optionally substituted with one or more halo;
Z is selected from C₆₋₁₀ aryl, 5- to 10-membered heteroaryl and 5- to 10-membered heterocycloalkyl, wherein the C₆₋₁₀ aryl, 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy.

The prodrug is typically an ester, an amide, a hydrazone or a disulfide of the compound of formula (10).

2. A compound according to paragraph 1, wherein:
R³ is selected from a group represented by formula (2), —[C(R⁷)₂]ₙ—X and —CH═CR⁶R⁷; and
X is selected from halo and —OH.
   3. A compound according to paragraph 1 or paragraph 2, which is represented by formula (10a):

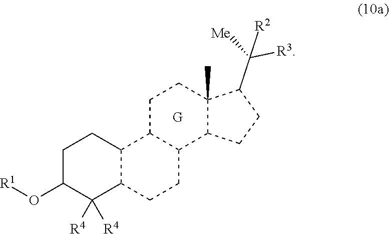

4. A compound according to paragraph 1 or paragraph 2, which is represented by formula (10b):

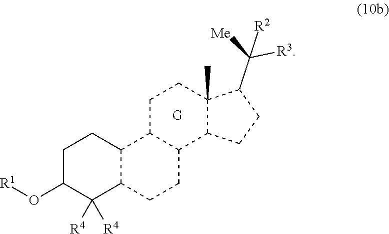

5. A compound according to paragraph 1 or paragraph 2, which is represented by formula (10p):

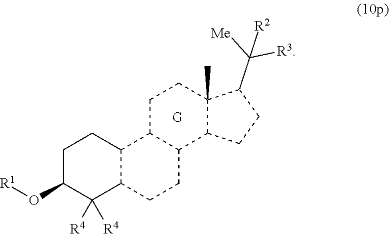

6. A compound according to paragraph 1 or paragraph 2, which is represented by formula (10q):

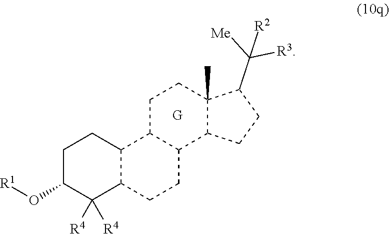

7. A compound according to any one of paragraphs 1 to 3 or 5, which is represented by formula (10ap):

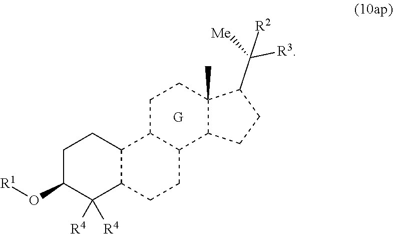

8. A compound according to any one of paragraphs 1 to 3 or 6, which is represented by formula (10aq):

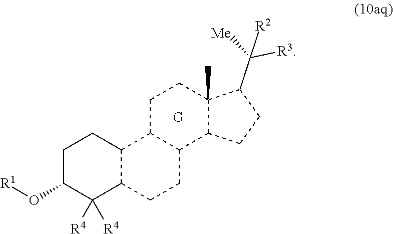

9. A compound according to any one of paragraphs 1, 2, 4 or 5, which is represented by formula (10 bp):

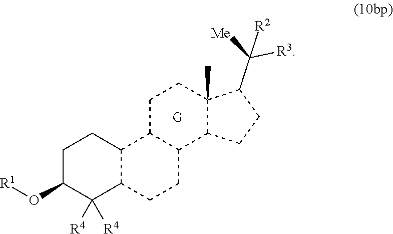

10. A compound according to any one of paragraphs 1, 2, 4 or 6, which is represented by formula (10bq):

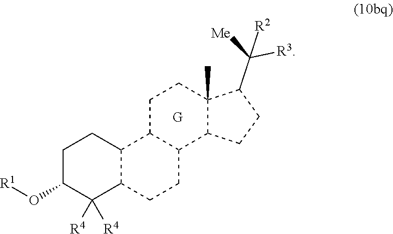

11. A compound according to any one of paragraphs 1 to 10, wherein R¹ is hydrogen.
12. A compound according to any one of paragraphs 1 to 10, wherein R¹ is —C(═O)R⁶.
13. A compound according to any one of paragraphs 1 to 12, wherein when R¹ is —C(═O)R⁶, then the R⁶ is C₁₋₆ alkyl (e.g. R¹ is —C(═O)—C₁₋₆ alkyl).
14. A compound according to paragraph 13, wherein the R⁶ is methyl or ethyl (e.g. R¹ is —C(═O)Me or —C(═O)Et).
15. A compound according to paragraph 14, wherein the R⁶ is methyl (e.g. R¹ is —C(═O)-Me).
16. A compound according to any one of paragraphs 1 to 15, wherein G is:

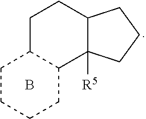

17. A compound according to paragraph 16, wherein G is:

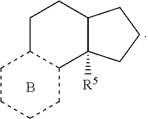

18. A compound according to any one of paragraphs 1 to 17, wherein R⁵ is selected from hydrogen, methyl and ethyl.
19. A compound according to paragraph 18, wherein R⁵ is selected from hydrogen and methyl.
20. A compound according to any one of paragraphs 1 to 19, wherein R⁵ is hydrogen.
21. A compound according to any one of paragraphs 1 to 19, wherein R⁵ is methyl.
22. A compound according to any one of paragraphs 1 to 21, wherein each R⁴ is independently selected from hydrogen, methyl and ethyl.
23. A compound according to any one of paragraphs 1 to 22, wherein each R⁴ is independently selected from hydrogen and methyl.
24. A compound according to any one of paragraphs 1 to 23, wherein at least one R⁴ is hydrogen.
25. A compound according to any one of paragraphs 1 to 24, wherein at least one R⁴ is methyl.
26. A compound according to any one of paragraphs 1 to 24, wherein both R⁴ are hydrogen.
27. A compound according to any one of paragraphs 1 to 23, wherein both R⁴ are methyl.
28. A compound according to any one of paragraphs 1 to 27, wherein B is selected from:

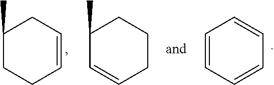

29. A compound according to any one of paragraphs 1 to 28, wherein B is:

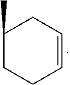

30. A compound according to any one of paragraphs 1 to 28, wherein B is:

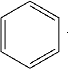

31. A compound according to any one of paragraphs 1 to 30, wherein R² is hydrogen.
32. A compound according to any one of paragraphs 1 to 31, wherein R³ is a group represented by formula (2):

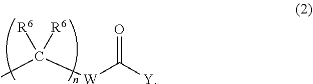

33. A compound according to any one of paragraphs 1 to 32, wherein when R³ is a group represented by formula (2), then each R⁶ is independently selected from hydrogen, methyl and ethyl.
34. A compound according to any one of paragraphs 1 to 33, wherein when R³ is a group represented by formula (2), then at least one R⁶ is hydrogen.
35. A compound according to any one of paragraphs 1 to 34, wherein R³ is a group represented by formula (2a):

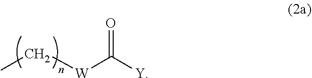

36. A compound according to any one of paragraphs 1 to 35, wherein when R³ is a group represented by formula (2) or formula (2a) and when W is NR⁶, then the R⁶ is selected from hydrogen, methyl and ethyl (e.g. W is selected from NH, NMe and NEt).
37. A compound according to paragraph 36, wherein the R⁶ is selected from hydrogen and methyl (e.g. W is selected from NH and NMe).
38. A compound according to paragraph 36 or paragraph 37, wherein the R⁶ is hydrogen (e.g. W is NH).
39. A compound according to paragraph 36 or paragraph 37, wherein the R⁶ is methyl (e.g. W is NMe).
40. A compound according to any one of paragraphs 1 to 39, wherein when R³ is a group represented by formula (2) or formula (2a), Y is selected from hydrogen, C₂ alkenyl and halo-C₂ alkenyl. The halo-C₂ alkenyl is C₂ alkenyl substituted with one or more halo.
41. A compound according to paragraph 40, wherein the halo-C₂ alkenyl is C₂ alkenyl substituted with one, two or three halo.
42. A compound according to paragraph 41, wherein the halo-C₂ alkenyl is C₂ alkenyl substituted with one halo.
43. A compound according to any one of paragraphs 40 to 42, wherein the halo of the halo-C₂ alkenyl is each independently selected from chloro (—Cl), bromo (—Br) and fluoro (—F).
44. A compound according to paragraph 43, wherein the halo of the halo-C₂ alkenyl is fluoro (—F).
45. A compound according to paragraph 40, wherein Y is hydrogen.
46. A compound according to paragraph 40, wherein Y is C₂ alkenyl.
47. A compound according to paragraph 40, wherein Y is halo-C₂ alkenyl (e.g. Y is C₂ alkenyl substituted with one or more halo groups).
48. A compound according to any one of paragraphs 1 to 31, wherein R³ is —[C(R⁷)₂]ₙ—X.
49. A compound according to any one of paragraphs 1 to 31 or 48, wherein when R³ is —[C(R⁷)₂]ₙ—X, then each R⁷ is independently selected from hydrogen, C₁₋₆ alkyl, halo-C₁₋₆ alkyl, C₂₋₆ alkenyl and halo-C₂₋₆ alkenyl. The halo-C₁₋₆ alkyl is C₁₋₆ alkyl substituted with one or more halo. The halo-C₂₋₆ alkenyl is C₂₋₆ alkenyl substituted with one or more halo.
50. A compound according to paragraph 49, wherein the halo-C₁₋₆ alkyl is C₁₋₆ alkyl substituted with one, two or three halo.
51. A compound according to paragraph 50, wherein the halo-C₁₋₆ alkyl is C₁₋₆ alkyl substituted with one halo.
52. A compound according to paragraph 49, wherein the halo-C₂₋₆ alkenyl is C₂₋₆ alkenyl substituted with one, two or three halo.
53. A compound according to paragraph 52, wherein the halo-C₂₋₆ alkenyl is C₂₋₆ alkenyl substituted with one halo.
54. A compound according to any one of paragraphs 49 to 53, wherein the halo of the halo-C₁₋₆ alkyl and the halo-C₂₋₆ alkenyl is each independently selected from chloro (—Cl), bromo (—Br) and fluoro (—F).
55. A compound according to paragraph 54, wherein the halo of the halo-C₁₋₆ alkyl and the halo-C₂₋₆ alkenyl is fluoro (—F).
56. A compound according to any one of paragraphs 1 to 55, wherein when R³ is —[C(R⁷)₂]ₙ—X, then each R⁷ is independently selected from hydrogen, C₁₋₆ alkyl, and C₂₋₆ alkenyl. The C₁₋₆ alkyl is unsubstituted. The C₂₋₆ alkenyl is unsubstituted.
57. A compound according to any one of paragraphs 1 to 55, wherein when R³ is —[C(R⁷)₂]ₙ—X, then at least one R⁷ on each carbon atom is hydrogen and the other R⁷ on each carbon atom is selected from hydrogen, C₁₋₆ alkyl, halo-C₁₋₆ alkyl, C₂₋₆ alkenyl and halo-C₂₋₆ alkenyl (e.g. R³ is —[CHR⁷], —X).
58. A compound according to paragraph 57, wherein when R³ is —[C(R⁷)₂]ₙ—X, then at least one R⁷ on each carbon atom is hydrogen and the other R⁷ on each carbon atom is selected from hydrogen, C₁₋₆ alkyl and C₂₋₆ alkenyl.
59. A compound according to paragraph 58, wherein when R³ is —[C(R⁷)₂]ₙ—X, then at least one R⁷ on each carbon atom is hydrogen and at least one R⁷ is selected from C₁₋₆ alkyl and C₂₋₆ alkenyl.
60. A compound according to paragraph 59, wherein when R³ is —[C(R⁷)₂]ₙ—X, then at least one R⁷ on each carbon atom is hydrogen and one of the other R⁷ is C₂₋₆ alkenyl.
61. A compound according to any one of paragraphs 1 to 55, wherein when R³ is —[C(R⁷)₂]ₙ—X, then all R⁷ are hydrogen (e.g. R³ is —[CH₂]ₙ—X).
62. A compound according to any one of paragraphs 1 to 61, wherein when R³ is —[C(R⁷)₂]ₙ—X, X is selected from halo and —OH. It is preferred that X is selected from fluoro (—F) and —OH.
63. A compound according to any one of paragraphs 1 to 61, wherein when R³ is —[C(R⁷)₂]ₙ—X and X is halo, then X is chloro (—Cl), bromo (—Br) or fluoro (—F).
64. A compound according to paragraph 63, wherein X is fluoro (—F).
65. A compound according to any one of paragraphs 1 to 31 or 48 to 62, wherein X is —OH.
66. A compound according to any one of paragraphs 1 to 31 or 48 to 61, wherein X is —SH.
67. A compound according to any one of paragraphs 1 to 31 or 48 to 61, wherein when R³ is —[C(R⁷)₂]ₙ—X and X is —O—Z, —S—Z or —S—S—Z, then Z is selected from C₆₋₁₀ aryl, 5- to 10-membered heteroaryl and 5- to 10-membered heterocycloalkyl, wherein the 5- to 10-membered heteroaryl and 5- to 10-membered heterocycloalkyl each contain at least one nitrogen atom.
68. A compound according to any one of paragraphs 1 to 31, 48 to 61 or 67, wherein when R³ is —[C(R⁷)₂]ₙ—X and X is —O—Z, —S—Z or —S—S—Z, then Z is selected from C₆₋₁₀ aryl and 5- to 10-membered heteroaryl.
69. A compound according to any one of paragraphs 1 to 31, 48 to 61, 67 or 68, wherein when R³ is —[C(R⁷)₂]ₙ—X and X is —O—Z, —S—Z or —S—S—Z, then Z is selected from phenyl and 5-, 6-, 9- or 10-membered heteroaryl.
70. A compound according to any one of paragraphs 1 to 31, 48 to 61 or 67 to 69, wherein when R³ is —[C(R⁷)₂]ₙ—X and X is —O—Z, —S—Z or —S—S—Z, then Z is selected from phenyl and 6- or 9-membered heteroaryl.
71. A compound according to any one of paragraphs 1 to 31, 48 to 61 or 67 to 70, wherein when R³ is —[C(R⁷)₂]ₙ—X and X is —O—Z, —S—Z or —S—S—Z, then Z is selected from phenyl, pyridinyl and benzothiazolyl.
72. A compound according to any one of paragraphs 1 to 31, 48 to 61 or 67 to 71, wherein when Z is selected from C₆₋₁₀ aryl, 5- to 10-membered heteroaryl and 5- to 10-membered heterocycloalkyl, then the C₆₋₁₀ aryl, 5- to 10-membered heteroaryl and 5- to 10-membered heterocycloalkyl are optionally substituted by one or more groups selected from —OH, chloro, bromo, fluoro, —NH₂, C₁₋₆ alkyl and C₁₋₆ alkoxy.
73. A compound according to paragraph 72, wherein the C₆₋₁₀ aryl, 5- to 10-membered heteroaryl and 5- to 10-membered heterocycloalkyl are optionally substituted by one or more groups selected from —OH, —NH₂, C₁₋₆ alkyl and C₁₋₆ alkoxy.
74. A compound according to paragraph 73, wherein the C₆₋₁₀ aryl, 5- to 10-membered heteroaryl and 5- to 10-membered heterocycloalkyl are optionally substituted by one or more groups selected from —OH and —NH₂.
75. A compound according to any one of paragraphs 1 to 31, 48 to 61 or 67 to 74, wherein X is —O—Z, —S—Z or —S—S—Z.
76. A compound according to paragraph 75, wherein X is —O—Z.
77. A compound according to paragraph 75, wherein X is —S—Z.
78. A compound according to paragraph 75, wherein X is —S—S—Z.
79. A compound according to any one of paragraphs 1 to 31 or 48 to 61, wherein when R³ is —[C(R⁷)₂]ₙ—X, X is selected from 5- to 10-membered heteroaryl and 5- to 10-membered heterocycloalkyl, wherein the 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy.
80. A compound according to paragraph 79, wherein the 5- to 10-membered heteroaryl and 5- to 10-membered heterocycloalkyl each contain at least one nitrogen atom.
81. A compound according to paragraph 80, wherein X is 5- to 10-membered heteroaryl.
82. A compound according to paragraph 81, wherein X is 5-, 6-, 9- or 10-membered heteroaryl.
83. A compound according to paragraph 82, wherein X is 5- or 6-membered heteroaryl.
84. A compound according to paragraph 83, wherein X is triazolyl.
85. A compound according to any one of paragraphs 1 to 31, 48 to 61 or 79 to 84, wherein when X is selected from 5- to 10-membered heteroaryl and 5- to 10-membered heterocycloalkyl, then the 5- to 10-membered heteroaryl and 5- to 10-membered heterocycloalkyl are optionally substituted by one or more groups selected from —OH, chloro, bromo, fluoro, —NH₂, C₁₋₆ alkyl and C₁₋₆ alkoxy.
86. A compound according to paragraph 85, wherein the 5- to 10-membered heteroaryl and 5- to 10-membered heterocycloalkyl are optionally substituted by one or more groups selected from —OH, —NH₂, C₁₋₆ alkyl and C₁₋₆ alkoxy.
87. A compound according to paragraph 86, wherein the 5- to 10-membered heteroaryl and 5- to 10-membered heterocycloalkyl are optionally substituted by one or more groups selected from —OH and —NH₂.
88. A compound according to any one of paragraphs 1 to 31, wherein R³ is —CH═CR⁶R⁷.
89. A compound according to paragraph 88, wherein the R⁶ is H (e.g. R³ is —CH═CHR⁷).
90. A compound according to paragraph 88 or paragraph 89, wherein R₇ is selected from hydrogen, C₁₋₆ alkyl, halo-C₁₋₆ alkyl, C₂₋₆ alkenyl and halo-C₂₋₆ alkenyl. The halo-C₁₋₆ alkyl is C₁₋₆ alkyl substituted with one or more halo. The halo-C₂₋₆ alkenyl is C₂₋₆ alkenyl substituted with one or more halo.
91. A compound according to paragraph 90, wherein the halo-C₁₋₆ alkyl is C₁₋₆ alkyl substituted with one, two or three halo.
92. A compound according to paragraph 91, wherein the halo-C₁₋₆ alkyl is C₁₋₆ alkyl substituted with one halo.
93. A compound according to any one of paragraphs 90 to 92, wherein the halo-C₂₋₆ alkenyl is C₂₋₆ alkenyl substituted with one, two or three halo.
94. A compound according to paragraph 93, wherein the halo-C₂₋₆ alkenyl is C₂₋₆ alkenyl substituted with one halo.
95. A compound according to any one of paragraphs 90 to 94, wherein the halo of the halo-C₁₋₆ alkyl and the halo-C₂₋₆ alkenyl is each independently selected from chloro (—Cl), bromo (—Br) and fluoro (—F).
96. A compound according to paragraph 95, wherein the halo of the halo-C₁₋₆ alkyl and the halo-C₂₋₆ alkenyl is fluoro (—F).
97. A compound according to any one of paragraphs 90 to 96, wherein R₇ is selected from hydrogen, C₁₋₆ alkyl, C₂₋₆ alkenyl and halo-C₂₋₆ alkenyl.
98. A compound according to paragraph 97, wherein R₇ is halo-C₂₋₆ alkenyl.
99. A compound according to any one of paragraphs 1 to 31, wherein R³ is —CR⁷═N—N(R⁶)₂.
100. A compound according to paragraph 99, wherein the R⁷ is selected from H and C₁₋₆ alkyl.
101. A compound according to paragraph 100, wherein the R⁷ is H.
102. A compound according to any one of paragraphs 99 to 101, wherein both the R⁶ are hydrogen (e.g. R³ is —CR⁷═N—NH₂).
103. A compound according to any one of paragraphs 1 to 31, wherein R³ is selected from 5- to 10-membered heteroaryl and 5- to 10-membered heterocycloalkyl, wherein the 5- to 10-membered heteroaryl and 5- to 10-membered heterocycloalkyl each contain at least one nitrogen atom.
104. A compound according to paragraph 103, wherein R³ is selected from 5-, 6-, 9- or 10-membered heteroaryl and 5-, 6-, 9- or 10-membered heterocycloalkyl.
105. A compound according to paragraph 104, wherein R³ is selected from 5- or 6-membered heteroaryl and 5- or 6-membered heterocycloalkyl.
107. A compound according to paragraph 105, wherein R³ is 5- or 6-membered heterocycloalkyl.
108. A compound according to paragraph 107, wherein R³ is imidazolinyl.
109. A compound according to any one of paragraphs 1 to 31 or 103 to 108, wherein when R³ is selected from 5- to 10-membered heteroaryl and 5- to 10-membered heterocycloalkyl, then the 5- to 10-membered heteroaryl and 5- to 10-membered heterocycloalkyl are optionally substituted by one or more groups selected from —OH, —NH₂, C₁₋₆ alkyl and C₁₋₆ alkoxy.
110. A compound according to paragraph 109, wherein the 5- to 10-membered heteroaryl and 5- to 10-membered heterocycloalkyl are optionally substituted by one or more groups selected from —OH and —NH₂.
111. A compound according to any one of paragraphs 1 to 110, wherein each n is independently an integer selected from 1, 2, 3, 4 and 5 (e.g. when R³ is a group represented by formula (2) or formula (2a) or is —[C(R⁷)₂]ₙ—X).
112. A compound according to paragraph 111, wherein each n is independently an integer selected from 1, 2 and 3.
113. A compound according to paragraph 112, wherein n is 1 (e.g. when X is —OH).
114. A compound according to paragraph 112, wherein n is 3 (e.g. when X is —OH).
115. A compound according paragraph 112, wherein n is 2 (e.g. when R³ is formula (2)).
116. A compound according to any one of paragraphs 1 to 115, wherein the compound of formula (10) is not one of:

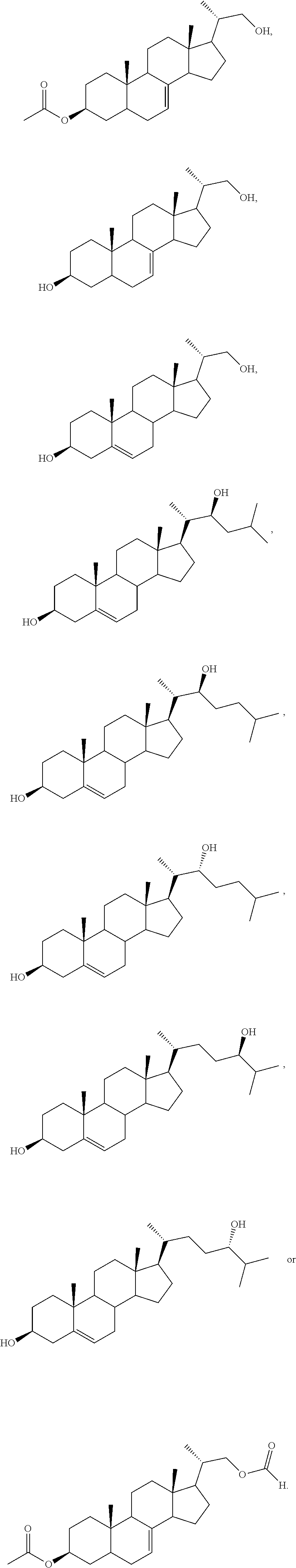

117. A compound according to any one of paragraphs 1 to 116 selected from:

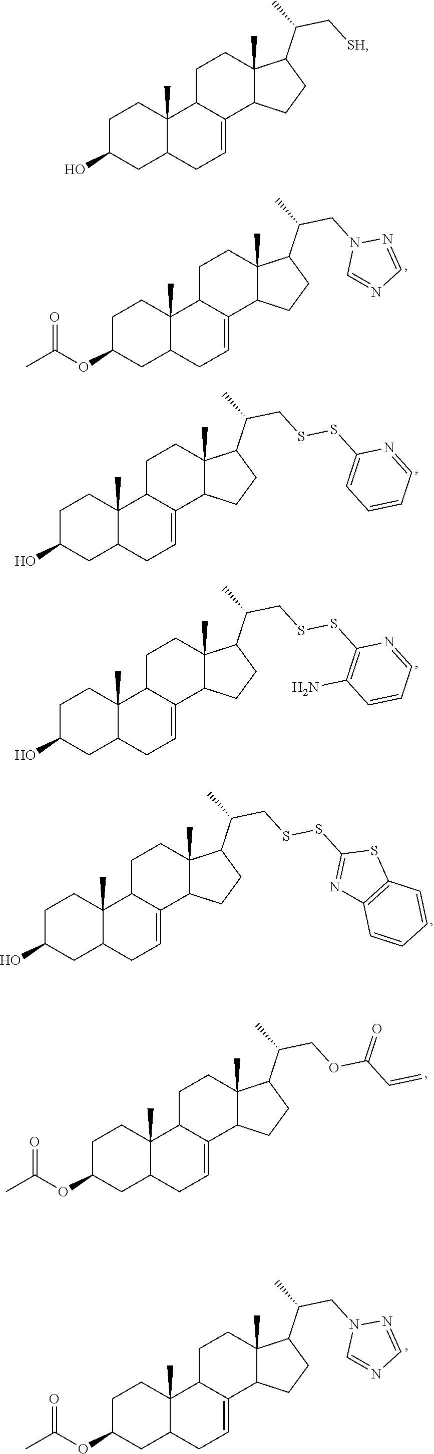

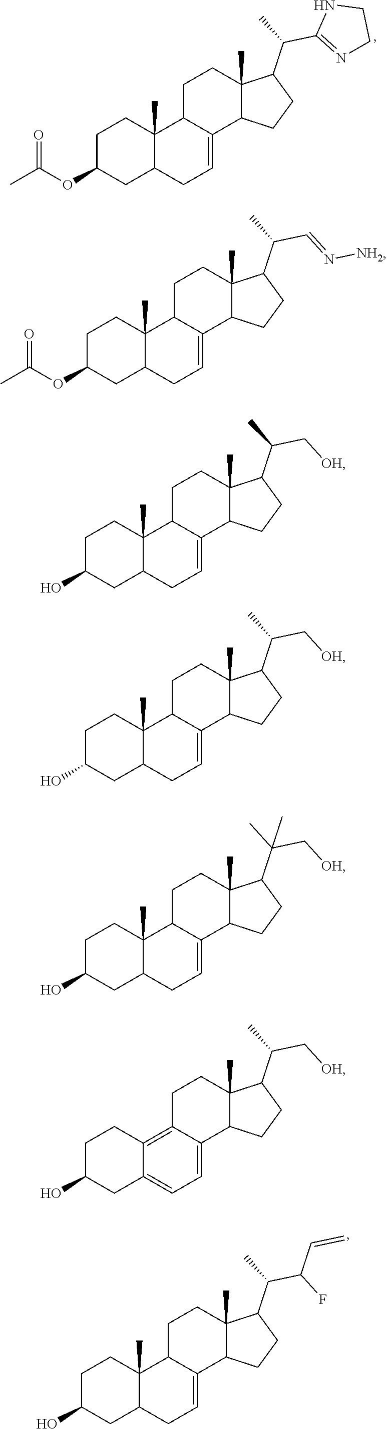

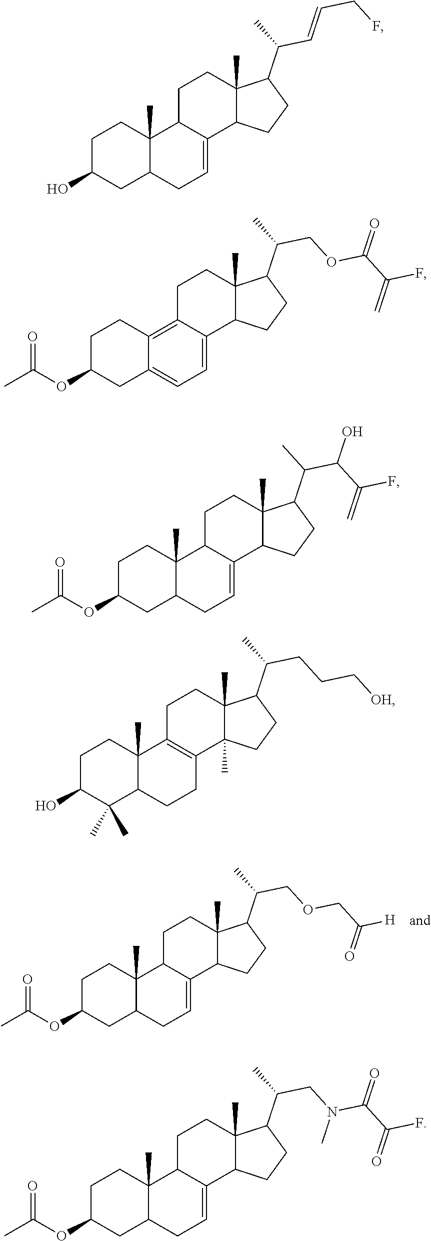

118. A compound according to any one of paragraphs 1 to 117, which is selected from:

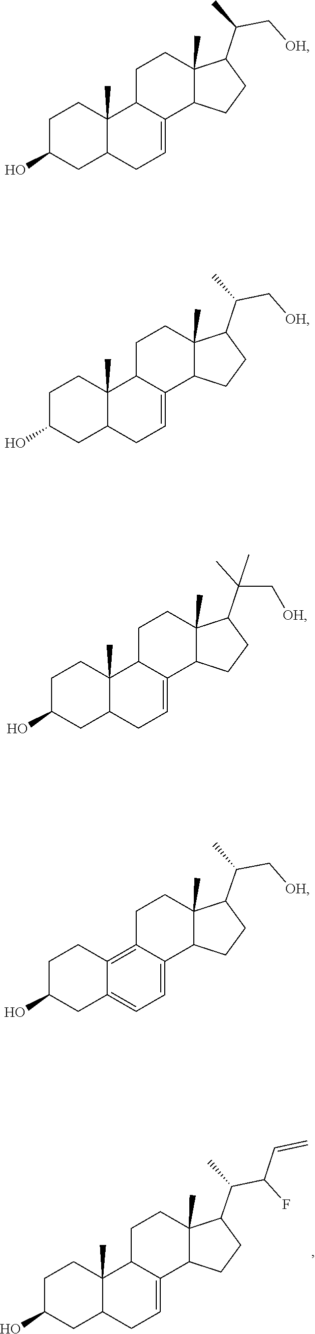 [SMILES: C[C@@H](CO)C1CCC2C3=CCC4C[C@@H](O)CC[C@]4(C)C3CC[C@@]21C ; C[C@H](CO)C1CCC2C3=CCC4C[C@H](O)CC[C@]4(C)C3CC[C@@]21C ; CC(C)(CO)C1CCC2C3=CCC4C[C@@H](O)CC[C@]4(C)C3CC[C@@]21C ; C[C@H](CO)C1CCC2c3ccc4c(c3CC[C@@]21C)CC[C@H](O)C4 ; C=CC(F)[C@@H](C)C1CCC2C3=CCC4C[C@@H](O)CC[C@]4(C)C3CC[C@@]21C]

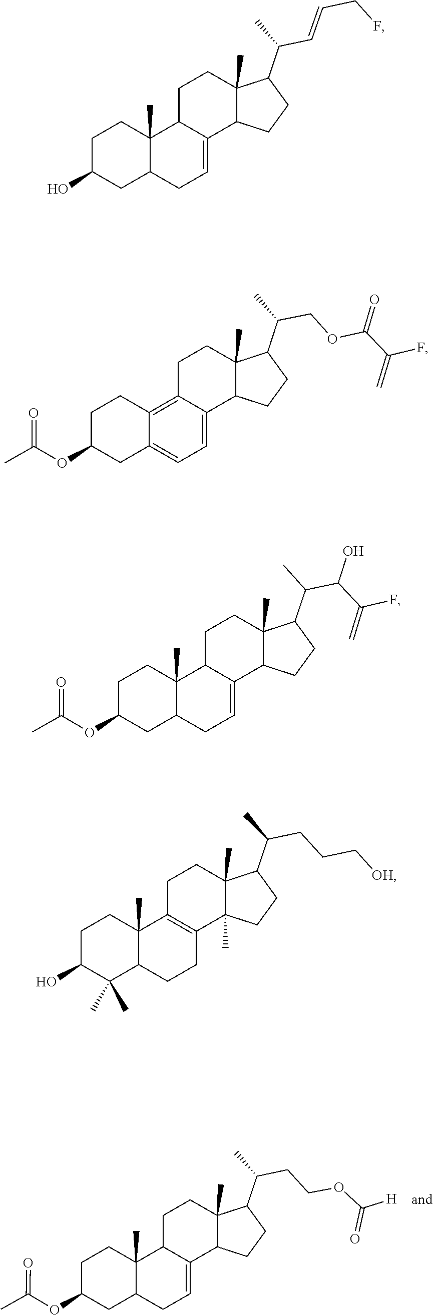

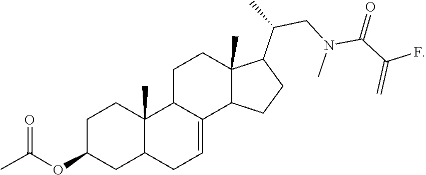 [SMILES: C=C(F)C(=O)N(C)C[C@@H](C)C1CCC2C3=CCC4C[C@@H](OC(C)=O)CC[C@]4(C)C3CC[C@@]21C]

The invention also provides a compound of formula (10), as defined in paragraphs 1 to 118 above, or a pharmaceutically acceptable salt, solvate, hydrate or prodrug thereof, for use in the treatment or prevention of a DHCR24-mediated disorder. This aspect of the invention also provides a method of treating or preventing a DHCR24-mediated disorder. The method comprises administering to a subject a compound of formula (10) or a pharmaceutically acceptable salt, solvate, hydrate or prodrug thereof.

The second aspect of the invention as described herein relates to a compound of formula (1) or a salt, solvate, hydrate or prodrug thereof. The salt is preferably a pharmaceutically acceptable salt.

### 2.1. In the Second Aspect of the Invention, the Compound of Formula (1) is:

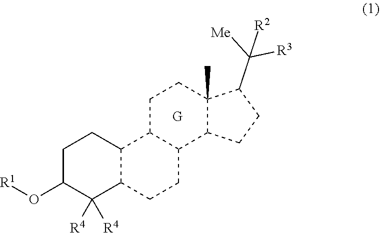

wherein:
G is a fused ring system selected from:

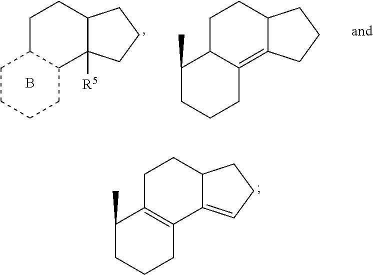

B is selected from:

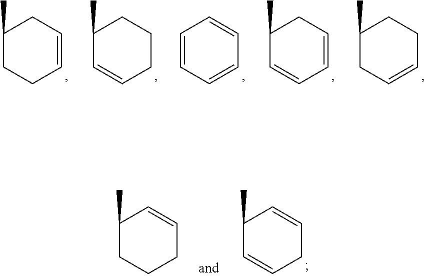

R¹ is selected from hydrogen, —C(═O)R⁶ and C₁₋₆ alkyl;
R² is selected from hydrogen and C₁₋₆ alkyl;
R³ is selected from a group represented by formula (2), —[C(R⁷)₂], —X, —C(═O)NHOR⁸, —C(═O)OR⁸, —CR⁷═N—N(R⁶)₂, —CH═CR⁸R⁷, C₆₋₁₀ aryl, 5- to 10-membered heteroaryl and 5- to 10-membered heterocycloalkyl, wherein the C₆₋₁₀ aryl, 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy;

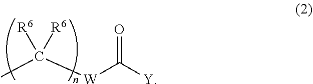

(2)
n is an integer from 1 to 6;
W is selected from O, S and NR⁶;
Y is selected from hydrogen, C₁₋₆ alkyl, C₂₋₆ alkenyl, amino-C₁₋₆ alkyl, (mono-C₁₋₆ alkylamino) C₁₋₆ alkyl and (di-C₁₋₆ alkylamino) C₁₋₆ alkyl, wherein the C₁₋₆ alkyl and C₂₋₆ alkenyl are optionally substituted with one or more halo;
X is selected from halo, —OH, —SH, —N(R⁶)₂, —O—Z, —S—Z, —S—S—Z, —C(═O)R⁷, C₆₋₁₀ aryl, 5- to 10-membered heteroaryl and 5- to 10-membered heterocycloalkyl, wherein the C₆₋₁₀ aryl, 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy;
each R⁴ is independently selected from hydrogen and C₁₋₆ alkyl;
R⁵ is selected from hydrogen and C₁₋₆ alkyl;
each R⁶ is independently selected from hydrogen and C₁₋₆ alkyl;
each R⁷ is independently selected from H, C₁₋₆ alkyl and C₂₋₆ alkenyl, wherein the C₁₋₆ alkyl and C₂₋₆ alkenyl are optionally substituted with one or more halo;
R⁸ is selected from C₁₋₆ alkyl and C₂₋₆ alkenyl; and
Z is selected from C₆₋₁₀ aryl, 5- to 10-membered heteroaryl and 5- to 10-membered heterocycloalkyl, wherein the C₆₋₁₀ aryl, 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy.
   2.2. A compound according to paragraph 2.1, which is represented by formula (Ia):

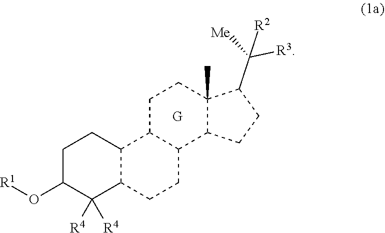

2.3. A compound according to paragraph 2.1, which is represented by formula (Ib):

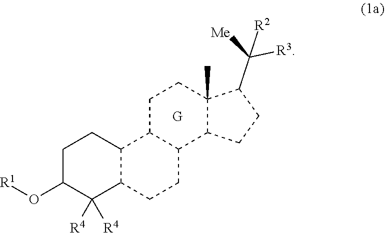

2.4. A compound according to paragraph 2.1, which is represented by formula (Ip):

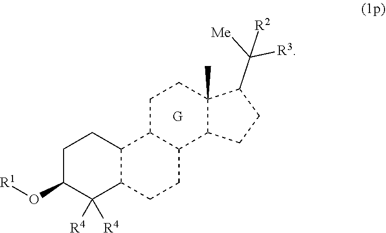

2.5. A compound according to paragraph 2.1, which is represented by formula (Iq):

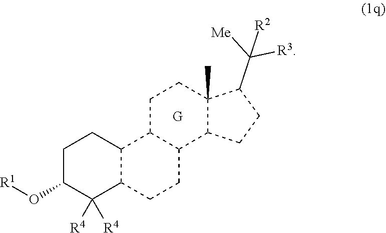

2.6. A compound according to any one of paragraphs 2.1, 2.2 or 2.4, which is represented by formula (1ap):

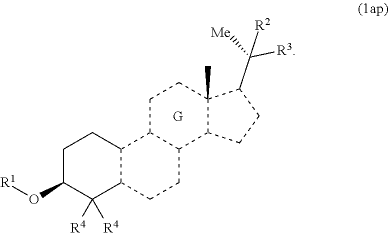

2.7. A compound according to any one of paragraphs 2.1, 2.2 or 2.5, which is represented by formula (1aq):

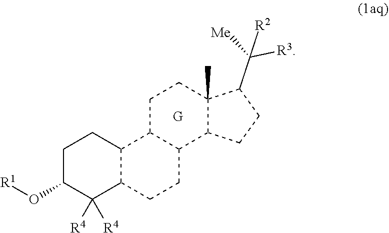

2.8. A compound according to any one of paragraphs 2.1, 2.3 or 2.4, which is represented by formula (1 bp):

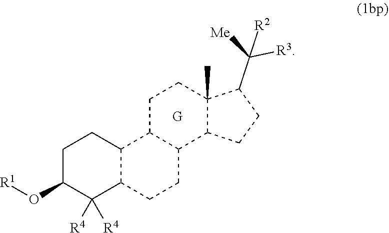

2.9. A compound according to any one of paragraphs 2.1, 2.3 or 2.5, which is represented by formula (1bq):

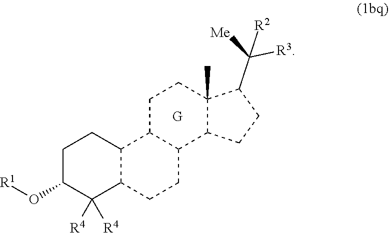

2.10. A compound according to any one of paragraphs 2.1 to 2.9, wherein R¹ is hydrogen or —C(═O)R⁶.
2.11. A compound according to any one of paragraphs 2.1 to 2.10, wherein R¹ is hydrogen.
2.12. A compound according to any one of paragraphs 2.1 to 2.10, wherein R¹ is —C(═O)R⁶.
2.13. A compound according to any one of paragraphs 2.1 to 2.12, wherein when R¹ is —C(═O)R⁶, then the R⁶ is C₁₋₆ alkyl (e.g. R⁶ is —C(═O)—C₁₋₆ alkyl).
2.14. A compound according to paragraph 2.13, wherein the Re is methyl or ethyl (e.g. R¹ is —C(═O)Me or —C(═O)Et).
2.15. A compound according to paragraph 2.14, wherein the R⁶ is methyl (e.g. R¹ is —C(═O)-Me).
2.16. A compound according to any one of paragraphs 2.1 to 2.15, wherein G is:

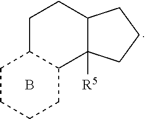

2.17. A compound according to paragraph 2.16, wherein G is:

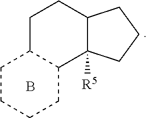

2.18. A compound according to any one of paragraphs 2.1 to 2.17, wherein R⁵ is selected from hydrogen, methyl and ethyl.
2.19. A compound according to paragraph 2.18, wherein R⁵ is selected from hydrogen and methyl.
2.20. A compound according to any one of paragraphs 2.1 to 2.19, wherein R⁵ is hydrogen.
2.21. A compound according to any one of paragraphs 2.1 to 2.19, wherein R⁵ is methyl.
2.22. A compound according to any one of paragraphs 2.1 to 2.21, wherein each R⁴ is independently selected from hydrogen, methyl and ethyl.
2.23. A compound according to any one of paragraphs 2.1 to 2.22, wherein each R⁴ is independently selected from hydrogen and methyl.
2.24. A compound according to any one of paragraphs 2.1 to 2.23, wherein at least one R⁴ is hydrogen.
2.25. A compound according to any one of paragraphs 2.1 to 2.24, wherein at least one R⁴ is methyl.
2.26. A compound according to any one of paragraphs 2.1 to 2.24, wherein both R⁴ are hydrogen.
2.27. A compound according to any one of paragraphs 2.1 to 2.23, wherein both R⁴ are methyl.
2.28. A compound according to any one of paragraphs 2.1 to 2.27, wherein B is selected from:

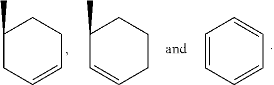

2.29. A compound according to any one of paragraphs 2.1 to 2.28, wherein B is:

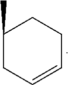

2.30. A compound according to any one of paragraphs 2.1 to 2.28, wherein B is:

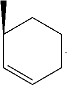

2.31. A compound according to any one of paragraphs 2.1 to 2.28, wherein B is:

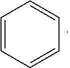

2.32. A compound according to any one of paragraphs 2.1 to 2.31, wherein R² is hydrogen.
2.33. A compound according to any one of paragraphs 2.1 to 2.32, wherein R³ is a group represented by formula (2):

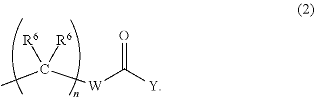

2.34. A compound according to any one of paragraphs 2.1 to 2.33, wherein when R³ is a group represented by formula (2), then each R⁶ is independently selected from hydrogen, methyl and ethyl.
2.35. A compound according to any one of paragraphs 2.1 to 2.34, wherein when R³ is a group represented by formula (2), then at least one R⁶ is hydrogen.
2.36. A compound according to any one of paragraphs 2.1 to 2.35, wherein R³ is a group represented by formula (2a):

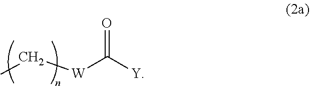

2.37. A compound according to any one of paragraphs 2.1 to 2.36, wherein when R³ is a group represented by formula (2) or formula (2a) and when W is NR⁶, then the R⁶ is selected from hydrogen, methyl and ethyl (e.g. W is selected from NH, NMe and NEt).
2.38. A compound according to paragraph 2.37, wherein the R⁶ is selected from hydrogen and methyl (e.g. W is selected from NH and NMe).
2.39. A compound according to paragraph 2.37 or paragraph 2.38, wherein the R⁶ is hydrogen (e.g. W is NH).
2.40. A compound according to paragraph 2.37 or paragraph 2.38, wherein the R⁶ is methyl (e.g. W is NMe).
2.41. A compound according to any one of paragraphs 2.1 to 2.40, wherein when R³ is a group represented by formula (2) or formula (2a), Y is selected from hydrogen, C₁₋₆ alkyl, halo-C₁₋₆ alkyl, C₂₋₆ alkenyl, halo-C₂₋₆ alkenyl, amino-C₁₋₆ alkyl, (mono-C₁₋₆ alkylamino) C₁₋₆ alkyl and (di-C₁₋₆ alkylamino) C₁₋₆ alkyl. The halo-C₁₋₆ alkyl is C₁₋₆ alkyl substituted with one or more halo. The halo-C₂₋₆ alkenyl is C₂₋₆ alkenyl substituted with one or more halo.
2.42. A compound according to paragraph 2.41, wherein the halo-C₁₋₆ alkyl is C₁₋₆ alkyl substituted with one, two or three halo.
2.43. A compound according to paragraph 2.42, wherein the halo-C₁₋₆ alkyl is C₁₋₆ alkyl substituted with one halo.
2.44. A compound according to any one of paragraphs 2.41 to 2.43, wherein the halo-C₂₋₆ alkenyl is C₂₋₆ alkenyl substituted with one, two or three halo.
2.45. A compound according to paragraph 2.44, wherein the halo-C₂₋₆ alkenyl is C₂₋₆ alkenyl substituted with one halo.
2.46. A compound according to any one of paragraphs 2.41 to 2.45, wherein the halo of the halo-C₁₋₆ alkyl and the halo-C₂₋₆ alkenyl is each independently selected from chloro (—Cl), bromo (—Br) and fluoro (—F).
2.47. A compound according to paragraph 2.46, wherein the halo of the halo-C₁₋₆ alkyl and the halo-C₂₋₆ alkenyl is fluoro (—F).
2.48. A compound according to any one of paragraphs 2.41 to 2.47, wherein R³ is a group represented by formula (2) or formula (2a), Y is selected from hydrogen, C₁₋₆ alkyl, C₂₋₆ alkenyl, halo-C₂₋₆ alkenyl, amino-C₁₋₆ alkyl, (mono-C₁₋₆ alkylamino) C₁₋₆ alkyl and (di-C₁₋₆ alkylamino) C₁₋₆ alkyl.
2.49. A compound according to any one of paragraphs 2.41 to 2.48, wherein Y is hydrogen.
2.50. A compound according to any one of paragraphs 2.41 to 2.48, wherein Y is C₁₋₆ alkyl.
2.51. A compound according to paragraph 2.50, wherein Y is C₁₋₃ alkyl.
2.52. A compound according to any one of paragraphs 2.41 to 2.48, wherein Y is C₂₋₆ alkenyl.
2.53. A compound according to paragraph 2.52, wherein Y is C₂₋₄ alkenyl.
2.54. A compound according to any one of paragraphs 2.41 to 2.48, wherein Y is halo-C₂₋₆ alkenyl (e.g. Y is C₂₋₆ alkenyl substituted with one or more halo groups).
2.55. A compound according to paragraph 2.54, wherein Y is halo-C₂₋₄ alkenyl.
2.56. A compound according to any one of paragraphs 2.41 to 2.48, wherein Y is selected from amino-C₁₋₆ alkyl, (mono-C₁₋₆ alkylamino) C₁₋₆ alkyl and (di-C₁₋₆ alkylamino) C₁₋₆ alkyl.
2.57. A compound according to paragraph 2.56, wherein Y is selected from amino-C₁₋₃ alkyl, (mono-C₁₋₃ alkylamino) C₁₋₃ alkyl and (di-C₁₋₃ alkylamino) C₁₋₃ alkyl.
2.58. A compound according to any one of paragraphs 2.1 to 2.36 or 2.41 to 2.57, wherein when R³ is a group represented by formula (2) or formula (2a), W is S.
2.59. A compound according to paragraph 2.58, wherein Y is hydrogen or C₁₋₆ alkyl.
2.60. A compound according to any one of paragraphs 2.1 to 2.57, wherein when R³ is a group represented by formula (3) or formula (3a), W is O or NR⁶.
2.61. A compound according to paragraph 2.60, wherein when R³ is a group represented by formula (3) or formula (3a), W is O.
2.62. A compound according to paragraph 2.60, wherein when R³ is a group represented by formula (3) or formula (3a), W is NR⁶.
2.63. A compound according to any one of paragraphs 2.1 to 2.32, wherein R³ is —[C(R⁷)₂]ₙ—X.
2.64. A compound according to any one of paragraphs 2.1 to 2.32 or 2.63, wherein when R³ is —[C(R⁷)₂]ₙ—X, then each R⁷ is independently selected from hydrogen, C₁₋₆ alkyl, halo-C₁₋₆ alkyl, C₂₋₆ alkenyl and halo-C₂₋₆ alkenyl. The halo-C₁₋₆ alkyl is C₁₋₆ alkyl substituted with one or more halo. The halo-C₂₋₆ alkenyl is C₂₋₆ alkenyl substituted with one or more halo.
2.65. A compound according to paragraph 2.64, wherein the halo-C₁₋₆ alkyl is C₁₋₆ alkyl substituted with one, two or three halo.
2.66. A compound according to paragraph 2.65, wherein the halo-C₁₋₆ alkyl is C₁₋₆ alkyl substituted with one halo.
2.67. A compound according to any one of paragraphs 2.64 to 2.66, wherein the halo-C₂₋₆ alkenyl is C₂₋₆ alkenyl substituted with one, two or three halo.
2.68. A compound according to paragraph 2.67, wherein the halo-C₂₋₆ alkenyl is C₂₋₆ alkenyl substituted with one halo.
2.69. A compound according to any one of paragraphs 2.64 to 2.68, wherein the halo of the halo-C₁₋₆ alkyl and the halo-C₂₋₆ alkenyl is each independently selected from chloro (—Cl), bromo (—Br) and fluoro (—F).
2.70. A compound according to paragraph 2.69, wherein the halo of the halo-C₁₋₆ alkyl and the halo-C₂₋₆ alkenyl is fluoro (—F).
2.71. A compound according to any one of paragraphs 2.1 to 2.32, 2.63 or 2.64, wherein when R³ is —[C(R⁷)₂]ₙ—X, then each R⁷ is independently selected from hydrogen, C₁₋₆ alkyl, and C₂₋₆ alkenyl. The C₁₋₆ alkyl is unsubstituted. The C₂₋₆ alkenyl is unsubstituted.
2.72. A compound according to any one of paragraphs 2.1 to 2.32 or 2.63 to 2.71, wherein when R³ is —[C(R⁷)₂]ₙ—X, then at least one R⁷ on each carbon atom is hydrogen and the other R⁷ on each carbon atom is selected from hydrogen, C₁₋₆ alkyl, halo-C₁₋₆ alkyl, C₂₋₆ alkenyl and halo-C₂₋₆ alkenyl (e.g. R³ is —[CHR⁷], —X).
2.73. A compound according to paragraph 2.72, wherein when R³ is —[C(R⁷)₂]ₙ—X, then at least one R⁷ on each carbon atom is hydrogen and the other R⁷ on each carbon atom is selected from hydrogen, C₁₋₆ alkyl and C₂₋₆ alkenyl.
2.74. A compound according to paragraph 2.73, wherein when R³ is —[C(R⁷)₂]ₙ—X, then at least one R⁷ on each carbon atom is hydrogen and at least one R⁷ is selected from C₁₋₆ alkyl and C₂₋₆ alkenyl.
2.75. A compound according to paragraph 2.74, wherein when R³ is —[C(R⁷)₂]ₙ—X, then at least one R⁷ on each carbon atom is hydrogen and one of the other R⁷ is C₂₋₆ alkenyl.
2.76. A compound according to any one of paragraphs 2.1 to 2.32 or 2.63 to 2.73, wherein when R³ is —[C(R⁷)₂]ₙ—X, then all R⁷ are hydrogen (e.g. R³ is —[CH₂]ₙ—X).
2.77. A compound according to any one of paragraphs 2.1 to 2.32 or 2.63 to 2.76, wherein when R³ is —[C(R⁷)₂]ₙ—X and X is halo, then X is chloro (—Cl), bromo (—Br) or fluoro (—F).
2.78. A compound according to paragraph 2.77, wherein X is fluoro (—F).
2.79. A compound according to any one of paragraphs 2.1 to 2.32 or 2.63 to 2.76, wherein X is —OH.
2.80. A compound according to any one of paragraphs 2.1 to 2.32 or 2.63 to 2.76, wherein X is —SH.
2.81. A compound according to any one of paragraphs 2.1 to 2.32 or 2.63 to 2.76, wherein when R³ is —[C(R⁷)₂]ₙ—X and X is —N(R⁶)₂, then each R⁶ is independently selected from hydrogen and C₁₋₃ alkyl.
2.82. A compound according to any one of paragraphs 2.1 to 2.32, 2.63 to 2.76 or 2.81, wherein when R³ is —[C(R⁷)₂]ₙ—X and X is —N(R⁶)₂, then at least one Re is hydrogen and the other R⁶ is selected from hydrogen and C₁₋₆ alkyl (e.g. X is —NHR⁶).
2.83. A compound according to paragraph 2.81 or 2.82, wherein both R⁶ are hydrogen (e.g. X is —NH₂).
2.84. A compound according to any one of paragraphs 2.1 to 2.32, 2.63 to 2.76 or 2.81 to 2.83, wherein X is —N(R⁶)₂.
2.85. A compound according to any one of paragraphs 2.1 to 2.32 or 2.63 to 2.76, wherein when R³ is —[C(R⁷)₂]ₙ—X and X is —O—Z, —S—Z or —S—S—Z, then Z is selected from C₆₋₁₀ aryl, 5- to 10-membered heteroaryl and 5- to 10-membered heterocycloalkyl, wherein the 5- to 10-membered heteroaryl and 5- to 10-membered heterocycloalkyl each contain at least one nitrogen atom.
2.86. A compound according to any one of paragraphs 2.1 to 2.32, 2.63 to 2.76 or 2.85, wherein when R³ is —[C(R⁷)₂]ₙ—X and X is —O—Z, —S—Z or —S—S—Z, then Z is selected from C₆₋₁₀ aryl and 5- to 10-membered heteroaryl.
2.87. A compound according to any one of paragraphs 2.1 to 2.32, 2.63 to 2.76, 2.85 or 2.86, wherein when R³ is —[C(R⁷)₂]ₙ—X and X is —O—Z, —S—Z or —S—S—Z, then Z is selected from phenyl and 5-, 6-, 9- or 10-membered heteroaryl.
2.88. A compound according to any one of paragraphs 2.1 to 2.32, 2.63 to 2.76 or 2.85 to 2.87, wherein when R³ is —[C(R⁷)₂]ₙ—X and X is —O—Z, —S—Z or —S—S—Z, then Z is selected from phenyl and 6- or 9-membered heteroaryl.
2.89. A compound according to any one of paragraphs 2.1 to 2.32, 2.63 to 2.76 or 2.85 to 2.88, wherein when R³ is —[C(R⁷)₂]ₙ—X and X is —O—Z, —S—Z or —S—S—Z, then Z is selected from phenyl, pyridinyl and benzothiazolyl.
2.90. A compound according to any one of paragraphs 2.1 to 2.32, 2.63 to 2.76 or 2.85 to 2.89, wherein when Z is selected from C₆₋₁₀ aryl, 5- to 10-membered heteroaryl and 5- to 10-membered heterocycloalkyl, then the C₆₋₁₀ aryl, 5- to 10-membered heteroaryl and 5- to 10-membered heterocycloalkyl are optionally substituted by one or more groups selected from —OH, chloro, bromo, fluoro, —NH₂, C₁₋₆ alkyl and C₁₋₆ alkoxy.
2.91. A compound according to paragraph 2.90, wherein the C₆₋₁₀ aryl, 5- to 10-membered heteroaryl and 5- to 10-membered heterocycloalkyl are optionally substituted by one or more groups selected from —OH, —NH₂, C₁₋₆ alkyl and C₁₋₆ alkoxy.
2.92. A compound according to paragraph 2.91, wherein the C₆₋₁₀ aryl, 5- to 10-membered heteroaryl and 5- to 10-membered heterocycloalkyl are optionally substituted by one or more groups selected from —OH and —NH₂.
2.93. A compound according to any one of paragraphs 2.1 to 2.32, 2.63 to 2.76 or 2.85 to 2.92, wherein X is —O—Z, —S—Z or —S—S—Z.
2.94. A compound according to paragraph 2.93, wherein X is —O—Z.
2.95. A compound according to paragraph 2.93, wherein X is —S—Z.
2.96. A compound according to paragraph 2.93, wherein X is —S—S—Z.
2.97. A compound according to any one of paragraphs 2.1 to 2.32 or 2.63 to 2.76, wherein X is —C(═O)R⁷.
2.98. A compound according to any one of paragraphs 2.1 to 2.32 or 2.63 to 2.76, wherein when R³ is —[C(R⁷)₂]ₙ—X, X is selected from C₆₋₁₀ aryl, 5- to 10-membered heteroaryl and 5- to 10-membered heterocycloalkyl, wherein the C₆₋₁₀ aryl, 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy.
2.99. A compound according to paragraph 2.98, wherein X is selected from C₆₋₁₀ aryl, 5- to 10-membered heteroaryl and 5- to 10-membered heterocycloalkyl, wherein the 5- to 10-membered heteroaryl and 5- to 10-membered heterocycloalkyl each contain at least one nitrogen atom.
2.100. A compound according to paragraph 2.99, wherein X is selected from C₆₋₁₀ aryl and 5- to 10-membered heteroaryl.
2.101. A compound according to paragraph 2.100, wherein X is selected from phenyl and 5-, 6-, 9- or 10-membered heteroaryl.
2.102. A compound according to paragraph 2.101, wherein X is 5- or 6-membered heteroaryl.
2.103. A compound according to paragraph 2.102, wherein X is triazolyl.
2.104. A compound according to any one of paragraphs 2.1 to 2.32, 2.63 to 2.76 or 2.98 to 2.103, wherein when X is selected from C₆₋₁₀ aryl, 5- to 10-membered heteroaryl and 5- to 10-membered heterocycloalkyl, then the C₆₋₁₀ aryl, 5- to 10-membered heteroaryl and 5- to 10-membered heterocycloalkyl are optionally substituted by one or more groups selected from —OH, chloro, bromo, fluoro, —NH₂, C₁₋₆ alkyl and C₁₋₆ alkoxy.
2.105. A compound according to paragraph 2.104, wherein the C₆₋₁₀ aryl, 5- to 10-membered heteroaryl and 5- to 10-membered heterocycloalkyl are optionally substituted by one or more groups selected from —OH, —NH₂, C₁₋₆ alkyl and C₁₋₆ alkoxy.
2.106. A compound according to paragraph 2.105, wherein the C₆₋₁₀ aryl, 5- to 10-membered heteroaryl and 5- to 10-membered heterocycloalkyl are optionally substituted by one or more groups selected from —OH and —NH₂.
2.107. A compound according to any one of paragraphs 2.1 to 2.32, wherein R³ is —C(═O)NHOR⁸.
2.108. A compound according to paragraph 2.107, wherein the R⁸ is C₁₋₆ alkyl.
2.109. A compound according to paragraph 2.108, wherein the R⁸ is C₁-3 alkyl.
2.110. A compound according to any one of paragraphs 2.1 to 2.32, wherein R³ is —C(═O)OR⁸.
2.111. A compound according to paragraph 2.110, wherein the R⁸ is C₂₋₆ alkenyl.
2.112. A compound according to any one of paragraphs 2.1 to 2.32, wherein R³ is —CR⁷═N—N(R⁶)₂.
2.113. A compound according to paragraph 2.112, wherein the R⁷ is selected from H and C₁₋₆ alkyl.
2.114. A compound according to paragraph 2.113, wherein the R⁷ is H.
2.115. A compound according to any one of paragraphs 2.112 to 2.114, wherein both the R⁶ are hydrogen (e.g. R³ is —CR⁷═N—NH₂).
2.116. A compound according to any one of paragraphs 2.1 to 2.32, wherein R³ is —CH═CR⁶R⁷.
2.117. A compound according to paragraph 2.116, wherein the R⁶ is H (e.g. R³ is —CH═CHR⁷).
2.118. A compound according to paragraph 2.116 or paragraph 2.117, wherein R₇ is selected from hydrogen, C₁₋₆ alkyl, halo-C₁₋₆ alkyl, C₂₋₆ alkenyl and halo-C₂₋₆ alkenyl. The halo-C₁₋₆ alkyl is C₁₋₆ alkyl substituted with one or more halo. The halo-C₂₋₆ alkenyl is C₂₋₆ alkenyl substituted with one or more halo.
2.119. A compound according to paragraph 2.118, wherein the halo-C₁₋₆ alkyl is C₁₋₆ alkyl substituted with one, two or three halo.
2.120. A compound according to paragraph 2.119, wherein the halo-C₁₋₆ alkyl is C₁₋₆ alkyl substituted with one halo.
2.121. A compound according to any one of paragraphs 2.118 to 2.120, wherein the halo-C₂₋₆ alkenyl is C₂₋₆ alkenyl substituted with one, two or three halo.
2.122. A compound according to paragraph 2.121, wherein the halo-C₂₋₆ alkenyl is C₂₋₆ alkenyl substituted with one halo.
2.123. A compound according to any one of paragraphs 2.118 to 2.122, wherein the halo of the halo-C₁₋₆ alkyl and the halo-C₂₋₆ alkenyl is each independently selected from chloro (—Cl), bromo (—Br) and fluoro (—F).
2.124. A compound according to paragraph 2.123, wherein the halo of the halo-C₁₋₆ alkyl and the halo-C₂₋₆ alkenyl is fluoro (—F).
2.125. A compound according to any one of paragraphs 2.118 to 2.124, wherein R₇ is selected from hydrogen, C₁₋₆ alkyl, C₂₋₆ alkenyl and halo-C₂₋₆ alkenyl.
2.126. A compound according to paragraph 2.125, wherein R₇ is halo-C₂₋₆ alkenyl.
2.127. A compound according to any one of paragraphs 2.1 to 2.32, wherein R³ is selected from C₆₋₁₀ aryl, 5- to 10-membered heteroaryl and 5- to 10-membered heterocycloalkyl, wherein the 5- to 10-membered heteroaryl and 5- to 10-membered heterocycloalkyl each contain at least one nitrogen atom.
2.128. A compound according to any one of paragraphs 2.1 to 2.32 or 2.127, wherein R³ is selected from 5- to 10-membered heteroaryl and 5- to 10-membered heterocycloalkyl.
2.129. A compound according to paragraph 2.128, wherein R³ is selected from 5-, 6-, 9- or 10-membered heteroaryl and 5-, 6-, 9- or 10-membered heterocycloalkyl.
2.130. A compound according to paragraph 2.129, wherein R³ is selected from 5- or 6-membered heteroaryl and 5- or 6-membered heterocycloalkyl.
2.131. A compound according to paragraph 2.130, wherein R³ is 5- or 6-membered heterocycloalkyl.
2.132. A compound according to paragraph 2.131, wherein R³ is imidazolinyl.
2.133. A compound according to any one of paragraphs 2.127 to 2.132, wherein the C₆₋₁₀ aryl, 5- to 10-membered heteroaryl and 5- to 10-membered heterocycloalkyl are optionally substituted by one or more groups selected from —OH, —NH₂, C₁₋₆ alkyl and C₁₋₆ alkoxy.
2.134. A compound according to paragraph 2.133, wherein the C₆₋₁₀ aryl, 5- to 10-membered heteroaryl and 5- to 10-membered heterocycloalkyl are optionally substituted by one or more groups selected from —OH and —NH₂.
2.135. A compound according to any one of paragraphs 2.1 to 2.134, wherein n is an integer selected from 1, 2, 3, 4 and 5.
2.136. A compound according to paragraph 2.135, wherein n is an integer selected from 1, 2 and 3.
2.137. A compound according to paragraph 2.136, wherein n is 1 (e.g. when X is —OH or —OZ). 2.138. A compound according to paragraph 2.136, wherein n is 3 (e.g. when X is —OH or —OZ).
2.139. A compound according paragraph 2.136, wherein n is 2 (e.g. when R³ is formula (2)).
2.140. A compound according to paragraph 2.1 selected from:

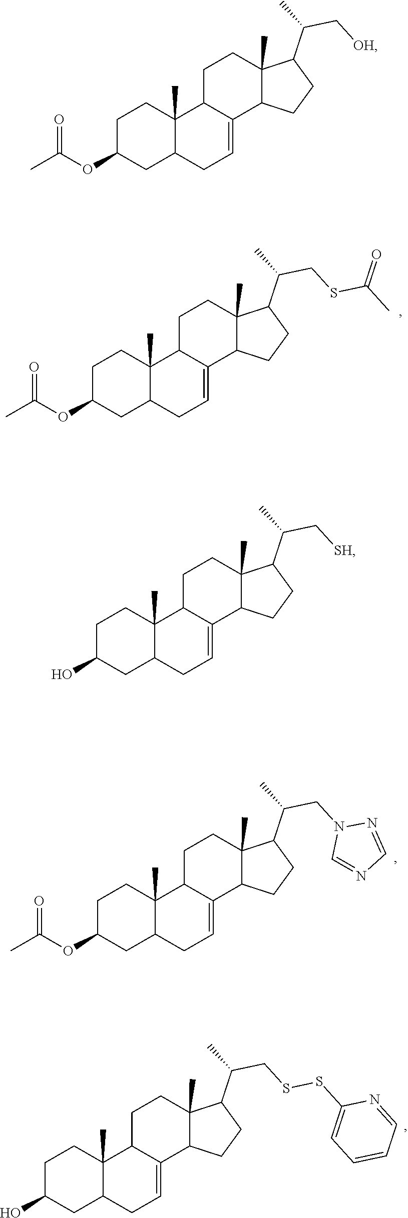

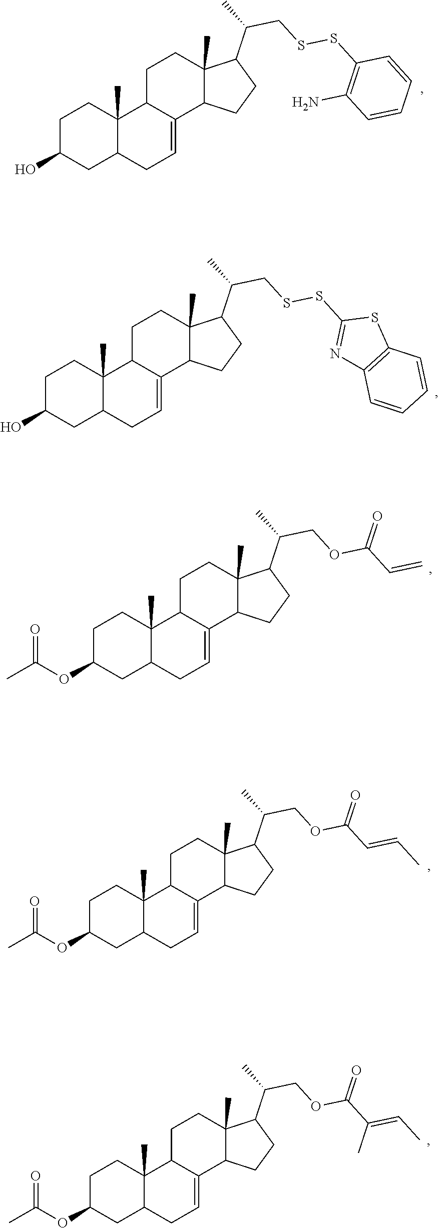

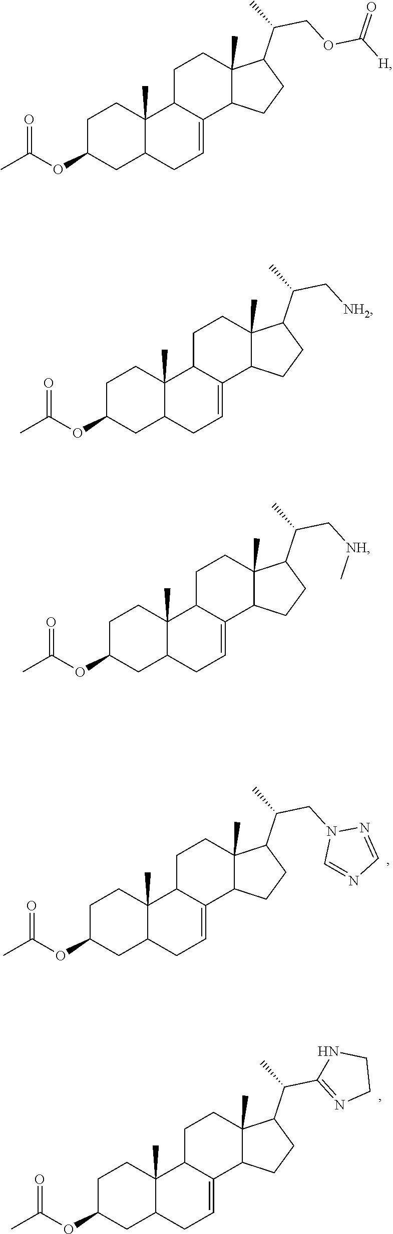

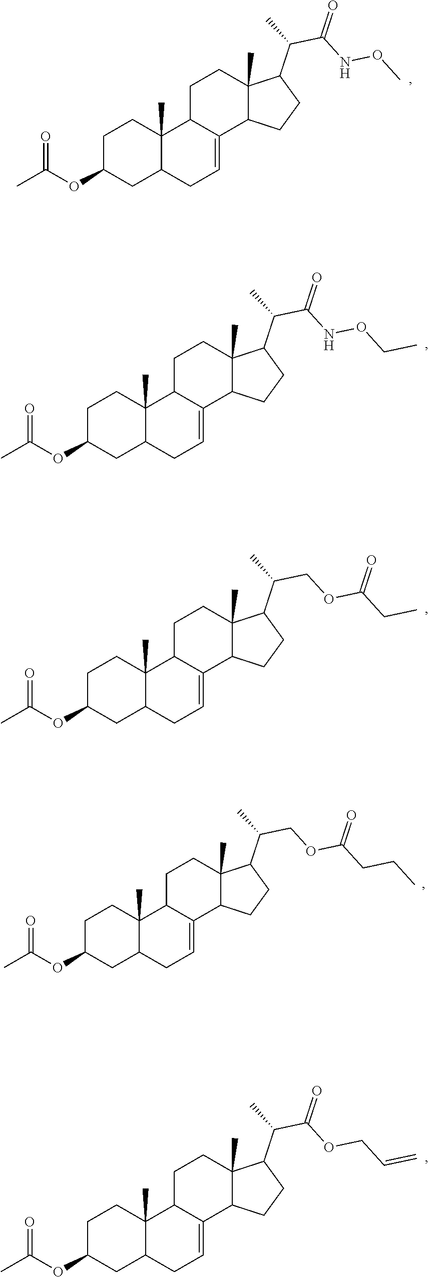

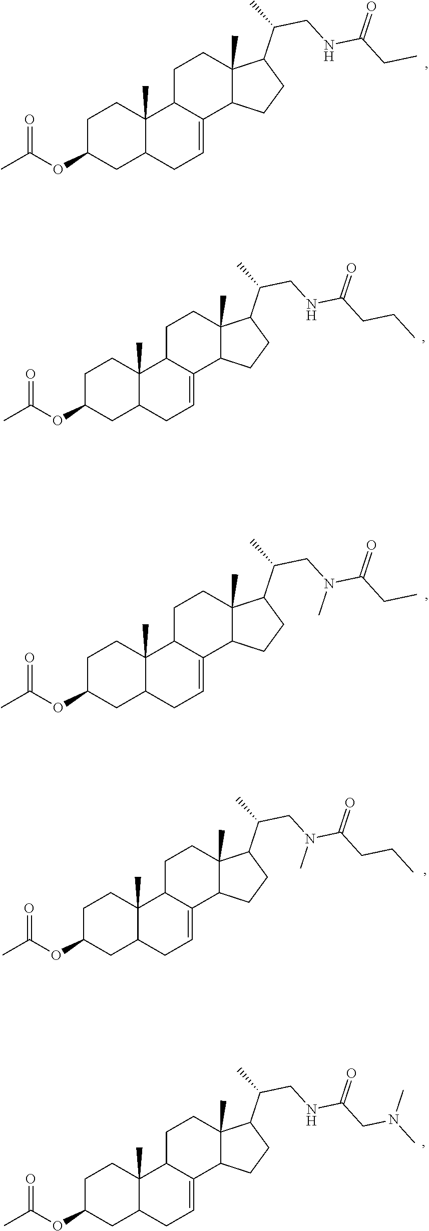

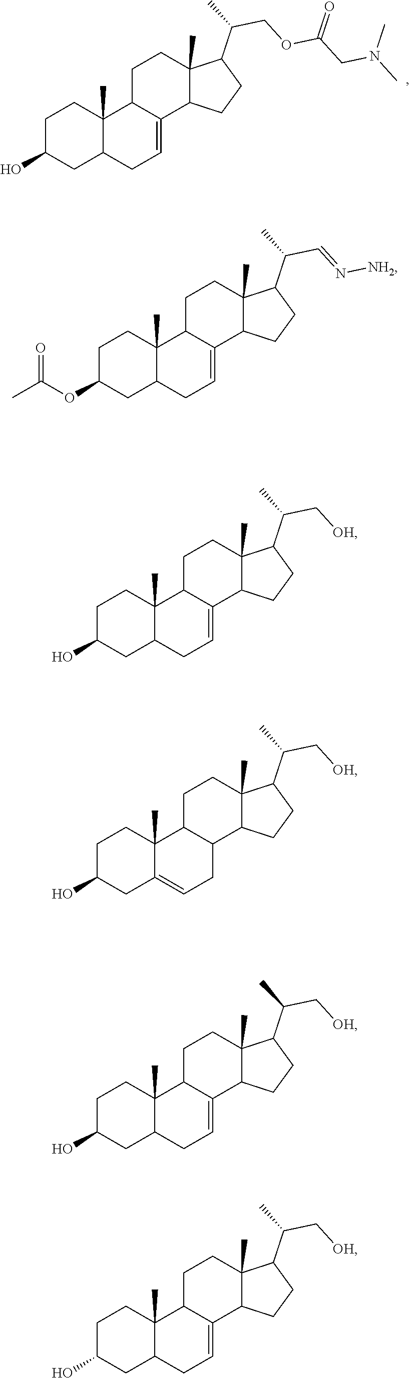

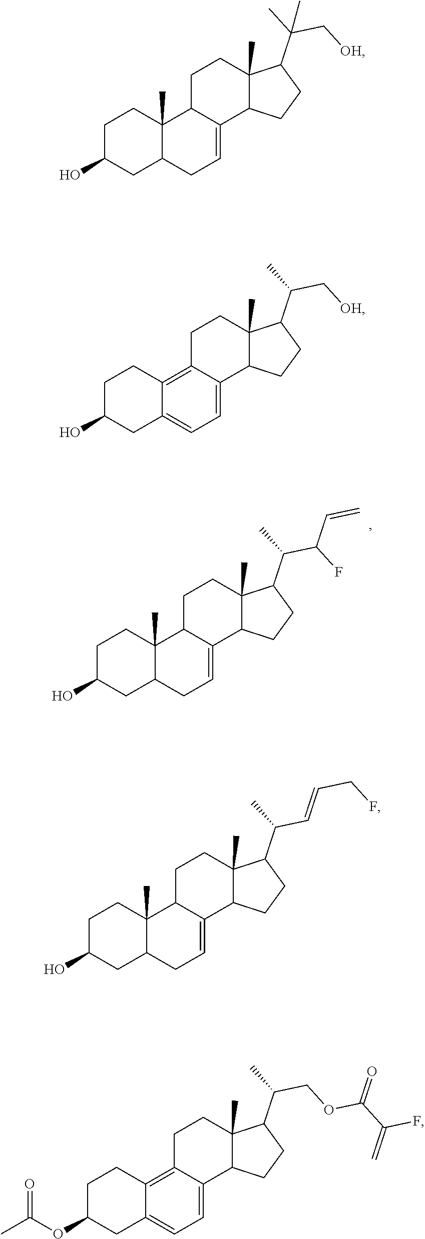

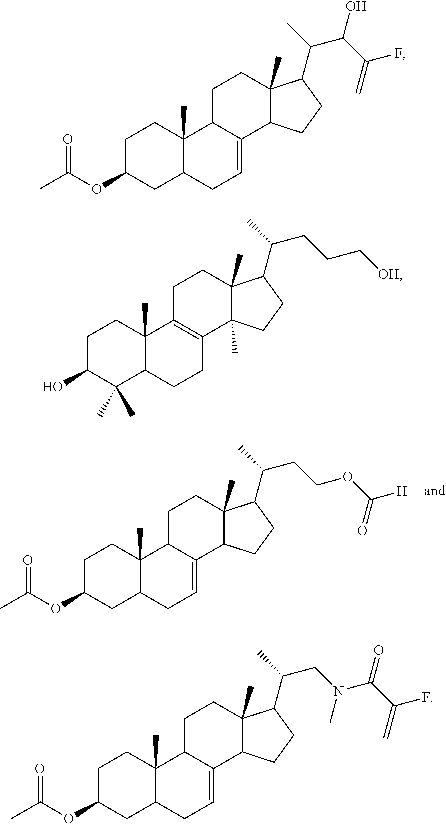

In the second aspect of the invention, the compound of formula (1) is preferably:

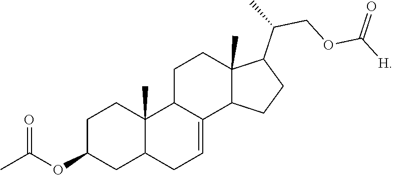

Another aspect of the invention is the provision of a pharmaceutical composition. The pharmaceutical composition comprises a compound of formula (1), such as defined in paragraphs 2.1 to 2.140 above, or a compound of formula (10), such as defined in paragraphs 1 to 118 above, or a pharmaceutically acceptable salt, solvate, hydrate or prodrug thereof, and a pharmaceutically acceptable excipient.

In another aspect, the invention provides a compound of formula (1), such as defined in paragraphs 2.1 to 2.140 above, or a compound of formula (10), such as defined in paragraphs 1 to 118 above, or a pharmaceutically acceptable salt, solvate, hydrate or prodrug thereof, or a pharmaceutical composition as defined herein, for use in therapy and/or for use as a medicament.

In another aspect, the present invention provides the use of the compound of formula (1), such as defined in paragraphs 2.1 to 2.140 above, or a compound of formula (10), such as defined in paragraphs 1 to 118 above, or a pharmaceutically acceptable salt, solvate, hydrate or prodrug thereof, in the manufacture of a medicament for the treatment or prevention of a DHCR24-mediated disorder.

A further aspect of the invention provides a method of inhibiting the activity of DHCR24 in vivo or in vitro. The in vivo or the in vitro method comprises contacting a cell with the compound of formula (1), such as defined in paragraphs 2.1 to 2.140 above, or a compound of formula (10), such as defined in paragraphs 1 to 118 above, or a pharmaceutically acceptable salt, solvate, hydrate or prodrug thereof, or a pharmaceutical composition as defined herein. Alternatively, the in vivo method may comprise administering to a subject a compound of formula (1), such as defined in paragraphs 2.1 to 2.140 above, or a compound of formula (10), such as defined in paragraphs 1 to 118 above, or a pharmaceutically acceptable salt, solvate, hydrate or prodrug thereof, or a pharmaceutical composition as defined herein.

In another aspect, the present invention provides a combination comprising a compound of formula (1), such as defined in paragraphs 2.1 to 2.140 above, or a compound of formula (10), such as defined in paragraphs 1 to 118 above, or a pharmaceutically acceptable salt, solvate, hydrate or prodrug thereof, with one or more additional therapeutic agents.

As a general feature of the invention, the DHCR24-mediated disorder may be selected from non-alcoholic steatohepatitis (NASH), atherosclerotic cardiovascular disease (asCVD), multiple sclerosis, hepatocellular carcinoma, breast cancer, endometrial carcinoma, B-cell lymphoma, drug-resistant cancer and hepatitis C. It is preferred that the DHCR24-mediated disorder is selected from non-alcoholic steatohepatitis (NASH), atherosclerotic cardiovascular disease (asCVD), multiple sclerosis (MS) and a combination of non-alcoholic steatohepatitis (NASH) and atherosclerotic cardiovascular disease (asCVD).

The DHCR24-mediated disorder is preferably non-alcoholic steatohepatitis (NASH).

The DHCR24-mediated disorder is preferably atherosclerotic cardiovascular disease (asCVD).

The DHCR24-mediated disorder is preferably multiple sclerosis (MS).

In general, the prodrug of a compound of formula (1), such as defined in paragraphs 2.1 to 2.140 above, or a compound of formula (10), such as defined in paragraphs 1 to 118 above, is an ester, an amide, a hydrazone or a disulfide of the compound of formula (1) or formula (10), respectively. It is preferred that the prodrug is an ester, an amide or a disulfide of the compound of formula (1) or formula (10). More preferably, the prodrug is an ester or an amide of the compound of formula (1) or formula (10).

When the prodrug is an ester or an amide, then the ester or amide group may be present at R¹ and/or R³ in formula (1) or formula (10) above, preferably at R¹. When the prodrug is a disulfide, then the disulfide group may be present at R³ in formula (1) or formula (10) above.

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Salts and Solvates

The compounds (including final products and intermediates) described herein may be isolated and used per se or may be isolated in the form of a salt, suitably pharmaceutically acceptable salts. It should be understood that the terms “salt(s)” and “salt form(s)” used by themselves or in conjunction with another term or terms encompasses all inorganic and organic salts, including industrially acceptable salts, as defined herein, and pharmaceutically acceptable salts, as defined herein, unless otherwise specified. As used herein, industrially acceptable salts are salts that are generally suitable for manufacturing and/or processing (including purification) as well as for shipping and storage, but may not be salts that are typically administered for clinical or therapeutic use. Industrially acceptable salts may be prepared on a laboratory scale, i.e. multi-gram or smaller, or on a larger scale, i.e. up to and including a kilogram or more.

Pharmaceutically acceptable salts, as used herein, are salts that are generally chemically and/or physically compatible with the other ingredients comprising a formulation, and/or are generally physiologically compatible with the recipient thereof. Pharmaceutically acceptable salts may be prepared on a laboratory scale, i.e. multi-gram or smaller, or on a larger scale, i.e. up to and including a kilogram or more. It should be understood that pharmaceutically acceptable salts are not limited to salts that are typically administered or approved by the FDA or equivalent foreign regulatory body for clinical or therapeutic use in humans. A practitioner of ordinary skill will readily appreciate that some salts are both industrially acceptable as well as pharmaceutically acceptable salts. It should be understood that all such salts, including mixed salt forms, are within the scope of the application.

In one embodiment, the compounds of formula (1) or formula (10) are isolated as pharmaceutically acceptable salts.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

In general, salts of the present application can be prepared in situ during the isolation and/or purification of a compound (including intermediates), or by separately reacting the compound (or intermediate) with a suitable organic or inorganic acid or base (as appropriate) and isolating the salt thus formed. The degree of ionisation in the salt may vary from completely ionised to almost non-ionised. In practice, the various salts may be precipitated (with or without the addition of one or more co-solvents and/or anti-solvents) and collected by filtration or the salts may be recovered by evaporation of solvent(s). Salts of the present application may also be formed via a “salt switch” or ion exchange/double displacement reaction, i.e. reaction in which one ion is replaced (wholly or in part) with another ion having the same charge. One skilled in the art will appreciate that the salts may be prepared and/or isolated using a single method or a combination of methods.

Representative salts include, but are not limited to, acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate and the like. Other examples of representative salts include alkali or alkaline earth metal cations such as sodium, lithium, potassium, calcium, magnesium, and the like, as well as non-toxic ammonium, quaternary ammonium and amine cations including, but not limited to, ammonium, tetramethylammonium, tetraethylammonium, lysine, arginine, benzathine, choline, tromethamine, diolamine, glycine, meglumine, oleamine and the like.

Certain compounds of the formula (1) or formula (10) may exist in solvated as well as unsolvated forms such as, for example, hydrated forms. It is to be understood that the invention encompasses all such solvated forms that possess antiproliferative activity.

### Polymorphs

It is also to be understood that certain compounds of the formula (1) or the formula (10) may exhibit polymorphism, and that the invention encompasses all such forms that possess antiproliferative activity.

### N-Oxides

Compounds of the formula (1) or formula (10) containing an amine function may also form N-oxides. A reference herein to a compound of the formula (1) or the formula (10) that contains an amine function also includes the corresponding N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

### Tautomers

Compounds of the formula (1) or formula (10) may exist in a number of different tautomeric forms and references to compounds of the formula (1) or the formula (10) include all such forms. For the avoidance of doubt, where a compound can exist in one of several tautomeric forms, and only one is specifically described or shown, all others are nevertheless embraced by formula (1) or formula (10). Examples of tautomeric forms include keto-, enol-, and enolate-forms, as in, for example, the following tautomeric pairs: keto/enol (illustrated below), pyrimidone/hydroxypyrimidine, imine/enamine, amide/imino alcohol, amidine/amidine, nitroso/oxime, thioketone/enethiol, and nitro/aci-nitro.

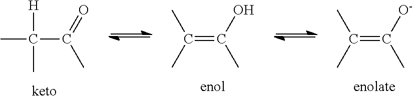

### Isomers

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is called a “racemic mixture”.

Certain compounds of formula (1) or formula (10) may have one or more asymmetric centers and therefore can exist in a number of stereoisomeric configurations. Consequently, such compounds can be synthesized and/or isolated as mixtures of enantiomers and/or as individual (pure) enantiomers, and, in the case of two or more asymmetric centers, single diastereomers and/or mixtures of diastereomers. It should be understood that the present application includes all such enantiomers and diastereomers and mixtures thereof in all ratios.

### Isotopes

The compounds of the present invention are described herein using structural formulas that do not specifically recite the mass numbers or the isotope ratios of the constituent atoms. As such it is intended that the present application includes compounds in which the constituent atoms are present in any ratio of isotope forms. For example, carbon atoms may be present in any ratio of ¹²C, ¹³C, and ¹⁴C; hydrogen atoms may be present in any ratio of ¹H, ²H, and ³H; etc. Preferably, the constituent atoms in the compounds of the present invention are present in their naturally occurring ratios of isotope forms.

### Prodrugs and Metabolites

The compounds of formula (1) or formula (10) may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the formula (1) or formula (10) and in-vivo cleavable amide derivatives that may be formed at a carboxy group or an amino group in a compound of the formula (1) or formula (10).

Accordingly, the present invention includes those compounds of formula (1) or formula (10) as defined hereinbefore when made available by organic synthesis and when made available within the human or animal body by way of cleavage of a pro-drug thereof. Accordingly, the present invention includes those compounds of formula (1) or formula (10) that are produced by organic synthetic means and also such compounds that are produced in the human or animal body by way of metabolism of a precursor compound, that is a compound of the formula (1) or formula (10) may be a synthetically-produced compound or a metabolically-produced compound.

A suitable pharmaceutically acceptable pro-drug of a compound of formula (1) or formula (10) is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of formula (1) or formula (10) that possesses a carboxy group is, for example, an in vivo cleavable ester thereof. The ester may be present at R¹ and/or in R³. An in vivo cleavable ester of a compound of formula (1) or formula (10) containing a carboxy group is, for example, a pharmaceutically acceptable ester which is cleaved in the human or animal body to produce the parent acid. Suitable pharmaceutically acceptable esters for carboxy include C₁₋₆ alkyl esters such as methyl, ethyl and tert-butyl, C₁₋₆ alkoxymethyl esters such as methoxymethyl esters, C₁₋₆alkanoyloxymethyl esters such as pivaloyloxymethyl esters, 3-phthalidyl esters, C₃₋₈cycloalkylcarbonyloxy-C₁₋₆ alkyl esters such as cyclopentylcarbonyloxymethyl and 1-cyclohexylcarbonyloxyethyl esters, 2-oxo-1,3-dioxolanylmethyl esters such as 5-methyl-2-oxo-1,3-dioxolen-4-ylmethyl esters and C₁₋₆ alkoxycarbonyloxy-C₁₋₆ alkyl esters such as methoxycarbonyloxymethyl and 1-methoxycarbonyloxyethyl esters.

A suitable pharmaceutically acceptable pro-drug of a compound of formula (1) or formula (10) that possesses a hydroxy group is, for example, an in vivo cleavable ester or ether thereof. The hydroxy group (—OH) may be present at R¹ and/or in R³. An in vivo cleavable ester or ether of a compound of formula (1) or formula (10) containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁₋₁₀ alkanoyl groups such as acetyl, benzoyl, phenylacetyl and substituted benzoyl and phenylacetyl groups, C₁₋₁₀ alkoxycarbonyl groups such as ethoxycarbonyl, N,N—(C₁₋₆)₂ carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4—(C₁₋₄ alkyl) piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of formula (1) or formula (10) that possesses a carboxy group is, for example, an in vivo cleavable amide thereof, for example an amide formed with an amine such as ammonia, a C₁₋₄ alkylamine such as methylamine, a (C₁₋₄ alkyl)₂ amine such as dimethylamine, N-ethyl-N-methylamine or diethylamine, a C₁₋₄ alkoxy-C₂₋₄ alkylamine such as 2-methoxyethylamine, a phenyl-C₁₋₄ alkylamine such as benzylamine and amino acids such as glycine or an ester thereof. These groups may be present at R¹ and/or in R³.

A suitable pharmaceutically acceptable pro-drug of a compound of formula (1) or formula (10) that possesses an amino group is, for example, an in vivo cleavable amide derivative thereof. Suitable pharmaceutically acceptable amides from an amino group include, for example an amide formed with C₁₋₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4—(C₁₋₄ alkyl) piperazin-1-ylmethyl. These groups may be present at R¹ and/or in R³.

The in vivo effects of a compound of formula (1) or formula (10) may be exerted in part by one or more metabolites that are formed within the human or animal body after administration of a compound of the formula (1) or formula (10), respectively. As stated hereinbefore, the in vivo effects of a compound of the formula (1) or formula (10) may also be exerted by way of metabolism of a precursor compound (a pro-drug).

### Pharmaceutical Compositions

According to a further aspect of the invention there is provided a pharmaceutical composition which comprises a compound of the invention as defined hereinbefore, or a pharmaceutically acceptable salt, solvate, hydrate or prodrug thereof, in association with a pharmaceutically acceptable excipient, such as a pharmaceutically acceptable diluent or a pharmaceutically acceptable carrier.

The pharmaceutical composition preferably comprises an effective amount, more preferably a therapeutically effective amount, of the compound of formula (1) or formula (10), or a pharmaceutically acceptable salt, solvate, hydrate or prodrug thereof.

The pharmaceutical composition may be formulated for delivery, particularly the selective delivery, to macrophages.

For example, the pharmaceutical composition may comprise a compound of the invention as defined hereinbefore, or a pharmaceutically acceptable salt, solvate, hydrate or prodrug thereof, in the form of nanoparticles.

In general, the compositions of the invention may be in a form suitable for oral use (for example as tablets (e.g. swallowable tablets), lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount, particularly a therapeutically effective amount, of a compound of the present invention for use in therapy is an amount sufficient to treat or prevent a DHCR24-mediated disorder referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula (1) or formula (10) will naturally vary according to the nature and severity of the conditions, the age and sex of the animal or patient and the route of administration, according to well known principles of medicine.

It is to be noted that dosages and dosing regimens may vary with the type and severity of the condition to be alleviated, and may include the administration of single or multiple doses, i.e. QD (once daily), BID (twice daily), etc., over a particular period of time (days or hours). It is to be further understood that for any particular subject or patient, specific dosage regimens may need to be adjusted over time according to the individual need and the professional judgment of the person administering or supervising the administration of the pharmaceutical compositions. For example, doses may be adjusted based on pharmacokinetic or pharmacodynamic parameters, which may include clinical effects such as toxic effects and/or laboratory values. Thus, the present application encompasses intra-patient dose-escalation as determined by the person skilled in the art. Procedures and processes for determining the appropriate dosage(s) and dosing regimen(s) are well-known in the relevant art and would readily be ascertained by the skilled artisan. As such, one of ordinary skill would readily appreciate and recognize that the dosage ranges set forth herein are exemplary only and are not intended to limit the scope or practice of the pharmaceutical compositions described herein.

In using a compound of the invention for therapeutic or prophylactic purposes it will generally be administered so that a daily dose in the range, for example, 0.1 mg/kg to 75 mg/kg body weight is received, given if required in divided doses. In general, lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

In another aspect, the present invention provides a compound of formula (1) or formula (10) as defined herein, or a pharmaceutically acceptable salt, solvate, hydrate or prodrug thereof, or a pharmaceutical composition as defined herein, for use in therapy and/or as a medicament. The therapy may be the treatment of the human or animal body.

In another aspect, the present invention provides a compound of formula (1) or formula (10) as defined herein, or a pharmaceutically acceptable salt, solvate, hydrate or prodrug thereof, or a pharmaceutical composition as defined herein, for use in the treatment or prevention of a DHCR24-mediated disorder.

In another aspect, the present invention provides a compound of formula (1) or formula (10) as defined herein, or a pharmaceutically acceptable salt, solvate, hydrate or prodrug thereof, or a pharmaceutical composition as defined herein, for use in the treatment or prevention of non-alcoholic steatohepatitis (NASH). The compound of formula (1) or formula (10) as defined herein, or a pharmaceutically acceptable salt, solvate, hydrate or prodrug thereof, or a pharmaceutical composition as defined herein, may be for use in the treatment or prevention of non-alcoholic steatohepatitis (NASH) without inducing hyperlipidemia and/or hypertriglyceridemia.

In another aspect, the present invention provides a compound of formula (1) or formula (10) as defined herein, or a pharmaceutically acceptable salt, solvate, hydrate or prodrug thereof, or a pharmaceutical composition as defined herein, for use in the treatment or prevention of atherosclerotic cardiovascular disease (asCVD). The compound of formula (1) or formula (10) as defined herein, or a pharmaceutically acceptable salt, solvate, hydrate or prodrug thereof, or a pharmaceutical composition as defined herein, may be for use in the treatment or prevention of atherosclerotic cardiovascular disease (asCVD) without inducing hyperlipidemia and/or hypertriglyceridemia.

In another aspect, the present invention provides a compound of formula (1) or formula (10) as defined herein, or a pharmaceutically acceptable salt, solvate, hydrate or prodrug thereof, or a pharmaceutical composition as defined herein, for use in the treatment or prevention of multiple sclerosis (MS).

In another aspect, the present invention provides the use of a compound of formula (1) or formula (10) as defined herein, or a pharmaceutically acceptable salt, solvate, hydrate or prodrug thereof, in the manufacture of a medicament for the treatment or prevention of a DHCR24-mediated disorder.

In another aspect, the present invention provides the use of a compound of formula (1) or formula (10) as defined herein, or a pharmaceutically acceptable salt, solvate, hydrate or prodrug thereof, in the manufacture of a medicament for the treatment or prevention of non-alcoholic steatohepatitis (NASH), preferably without inducing hyperlipidemia and/or hypertriglyceridemia.

In another aspect, the present invention provides the use of a compound of formula (1) or formula (10) as defined herein, or a pharmaceutically acceptable salt, solvate, hydrate or prodrug thereof, in the manufacture of a medicament for the treatment or prevention of atherosclerotic cardiovascular disease (asCVD), preferably without inducing hyperlipidemia and/or hypertriglyceridemia.

In another aspect, the present invention provides the use of a compound of formula (1) or formula (10) as defined herein, or a pharmaceutically acceptable salt, solvate, hydrate or prodrug thereof, in the manufacture of a medicament for the treatment or prevention of multiple sclerosis (MS).

In another aspect, the present invention provides a method of treating or preventing a DHCR24-mediated disorder, said method comprising administering to a subject in need thereof an effective amount of a compound of formula (1) or formula (10) as defined herein, or a pharmaceutically acceptable salt, solvate, hydrate or prodrug thereof, or a pharmaceutical composition as defined herein.

In another aspect, the present invention provides a method of treating or preventing non-alcoholic steatohepatitis (NASH), without inducing hyperlipidemia and/or hypertriglyceridemia, said method comprising administering to a subject in need thereof an effective amount of a compound of formula (1) or formula (10) as defined herein, or a pharmaceutically acceptable salt, solvate, hydrate or prodrug thereof, or a pharmaceutical composition as defined herein.

In another aspect, the present invention provides a method of treating or preventing atherosclerotic cardiovascular disease (asCVD), without inducing hyperlipidemia and/or hypertriglyceridemia, said method comprising administering to a subject in need thereof an effective amount of a compound of formula (1) or formula (10) as defined herein, or a pharmaceutically acceptable salt, solvate, hydrate or prodrug thereof, or a pharmaceutical composition as defined herein.

In another aspect, the present invention provides a method of treating or preventing multiple sclerosis (MS), said method comprising administering to a subject in need thereof an effective amount of a compound of formula (1) or formula (10) as defined herein, or a pharmaceutically acceptable salt, solvate, hydrate or prodrug thereof, or a pharmaceutical composition as defined herein.

A further aspect of the invention provides a method of inhibiting the activity of DHCR24 in vivo or in vitro.

The in vivo or the in vitro method comprises contacting a cell with the compound of formula (1) or formula (10), or a pharmaceutically acceptable salt, solvate, hydrate or prodrug thereof, or a pharmaceutical composition as defined herein.

Alternatively, the in vivo method may comprise administering to a subject in need thereof a therapeutically effective amount of a compound of formula (1) or formula (10), or a pharmaceutically acceptable salt, solvate, hydrate or prodrug thereof, or a pharmaceutical composition as defined herein.

In another aspect, the present invention provides a combination comprising a compound of formula (1) or formula (10), or a pharmaceutically acceptable salt, solvate, hydrate or prodrug thereof, with one or more additional therapeutic agents.

In each of the above aspects, the DHCR24-mediated disorder may be selected from non-alcoholic steatohepatitis (NASH), atherosclerotic cardiovascular disease (asCVD), multiple sclerosis (MS), hepatocellular carcinoma (HCC), breast cancer, endometrial carcinoma, B-cell lymphoma, drug-resistant cancer and hepatitis C.

Additionally or as alternative to the DHCR24-mediate disorders defined above, in each of the above aspects of the invention, the DHCR24-mediated disorder may be liver inflammation, liver fibrosis and/or liver injury. It is preferred that the DHCR24-mediated disorder is liver fibrosis and/or liver injury.

The breast cancer is preferably luminal breast cancer or HER2 positive breast cancer.

The drug-resistant cancer is preferably methotrexate resistant cancer. The cancer may be gestational trophoblastic neoplasia (GTN).

It is preferred that the DHCR24-mediated disorder is non-alcoholic steatohepatitis (NASH), atherosclerotic cardiovascular disease (asCVD) or multiple sclerosis (MS). The DHCR24-mediated disorder is preferably non-alcoholic steatohepatitis (NASH). The DHCR24-mediated disorder is preferably atherosclerotic cardiovascular disease (asCVD). The DHCR24-mediated disorder is preferably multiple sclerosis (MS).

Non-alcoholic steatohepatitis (NASH) and atherosclerotic cardiovascular disease (asCVD) cause massive morbidity and mortality. In fact, asCVD is the main cause of death in the Western world. Both diseases are driven by lipids/cholesterol and inflammation. asCVD is mainly treated by cholesterol-lowering statins, while no effective drugs for NASH currently exist, and NASH is mainly treated by lifestyle interventions. Besides lipid-lowering approaches, both diseases would highly benefit from a concomitant anti-inflammatory approach.

By inhibiting the enzyme 24-dehydrocholesterol reductase (DHCR24), the terminal step in the de novo synthesis of cholesterol is mediated. This is the step where the enzyme converts desmosterol into cholesterol. DHCR24 inhibition not only reduces cholesterol synthesis, but also induces an increase of desmosterol, which is the endogenous ligand of liver X receptor alpha (LXRα). Activation of LXRα results in an efficient anti-inflammatory effect, which dampens macrophage activation. Synthetic LXRα ligands in the prior art generate massive hypertriglyceridemia (another risk factor for asCVD) by inducing lipogenesis through SREBP in hepatocytes. In contrast, the increase in desmosterol elicited by the selective DHCR24 inhibitor compounds of the invention activate LXRα without side effects, such as inducing hypertriglyceridemia.

It has been found that desmosterol plays a critical role in resolving inflammation in demyelinated lesions in multiple sclerosis (see Nature Neuroscience, 2021, 24(1), pp. 47-60). The increase in desmosterol produced by the selective DHCR24 inhibitor compounds may be used in the treatment or prevention, preferably treatment, of multiple sclerosis.

It has been shown that overexpression of DHCR24 correlates with a poor clinical outcome in patients with hepatocellular carcinoma (see British Journal of Cancer, 2000, 123, pp. 1673-1685). DCHR24 inhibitors may therefore be useful in the prevention or treatment of HCC.

Cholesterol has been shown to be a risk factor for breast cancer. It has been shown that DHCR24 expression was higher in breast cancer, especially in luminal and HER2 positive breast cancer tissues compared with normal breast (see Cancer Sci., 2020, 111(10), pp. 3653-3664). Compounds that inhibit DHCR24 may therefore be useful in the treatment or prevention of breast cancer.

DHCR24 has also been associated with urogenital neoplasms. It has been shown that DHCR24 is significantly elevated in patients with endometrial cancer (EC), and that the up-regulation of DHCR24 was associated with advanced clinical stage, histological grading, vascular invasion, lymphatic metastasis, and reduced overall survival (see Scientific Reports, 2017, 7 (1), pp. 41404; DOI: 10.1038/srep41404). Compounds that inhibit DHCR24 may therefore be useful in the treatment or prevention of EC.

DHCR24 has been identified as being a direct target of SOX9, the oncogenic stem cell regulator, in B-cell lymphomas. It has been shown that SOX9 can drive lymphomagenesis through DHCR24 and the cholesterol biosynthesis pathway (see Blood, 2022, 139(1), pp. 73-86). Compounds that inhibit DHCR24 may therefore be useful in the treatment or prevention of B-cell lymphoma.

DHCR24 has been identified as a potential downstream effector of DPP4 (see Front. Oncol., 2 Dec. 2021; https://doi.org/10.3389/fonc.2021.704024). Targeting DPP4/DHCR24 signalling may help sensitize methotrexate-resistant cancer, particularly gestational trophoblastic neoplasia (GTN).

It has been shown that expression of DHCR24 in human hepatocytes was induced by the hepatitis C virus (HCV) infection (see J. Hepatol., 2011, 55(3), pp. 512-521). The inhibition of DHCR24 can decrease HCV replication. Thus, Compounds that inhibit DHCR24 may therefore be useful in the treatment or prevention of hepatitis C.

### Routes of Administration

The compounds or the pharmaceutical compositions of the invention may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapy

The treatment defined hereinbefore may be applied as a sole therapy or may involve, in addition to the compound of formula (1) or formula (10), the administration of one or more therapeutic agents.

The therapeutic agent may be a lipid-lowering agent, such as a statin, a PCSK9 inhibitor (e.g. Evolocumab, Alirocumab) or an NPC1L1 inhibitor (e.g. Ezetimibe), particularly when the DHCR24-mediated disorder is atherosclerotic cardiovascular disease (asCVD).

According to this aspect of the invention there is provided a combination for use in the treatment or prevention of a DHCR24-mediated disorder, particularly the treatment or the prevention of non-alcoholic steatohepatitis (NASH) or atherosclerotic cardiovascular disease (asCVD), comprising a compound of formula (1) or formula (10) as defined hereinbefore, or a pharmaceutically acceptable salt, solvate, hydrate or prodrug thereof, and another therapeutic agent, such as a therapeutic agent for treating or preventing non-alcoholic steatohepatitis (NASH) or atherosclerotic cardiovascular disease (asCVD).

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination. In one embodiment, a combination refers to a combination product.

According to a further aspect of the invention, there is provided a combination comprising a compound of formula (1) or formula (10) as defined hereinbefore, or a pharmaceutically acceptable salt, solvate, hydrate or prodrug thereof, and a therapeutic agent, such as described above.

In one embodiment, there is provided a pharmaceutical composition which comprises a compound of formula (1) or formula (10) as defined hereinbefore, or a pharmaceutically acceptable salt, solvate, hydrate or prodrug thereof, and another therapeutic agent, in combination with a therapeutic agent, such as described above, in association with a pharmaceutically acceptable diluent or carrier.

### EXAMPLES

The following examples are provided to illustrate the invention and are not intended to limit the scope of the invention, as described herein.

The compounds of the invention may be prepared using synthetic techniques that are known in the art (as illustrated by the examples herein).

Several methods for the chemical synthesis of the compounds of the present application are described herein. These and/or other well-known methods may be modified and/or adapted in various ways in order to facilitate the synthesis of additional compounds within the scope of the present application and claims. Such alternative methods and modifications should be understood as being within the spirit and scope of this application and claims. Accordingly, it should be understood that the methods set forth in the following descriptions, schemes and examples are intended for illustrative purposes and are not to be construed as limiting the scope of the disclosure.

### Synthesis of Compounds

The compounds were prepared from commercially available starting materials. The general reaction scheme shown in Scheme 1 exemplifies the published synthesis of versatile/reactive key intermediates starting from commercially available ergosterol, which can be used in the synthesis of most of the compounds set out below.

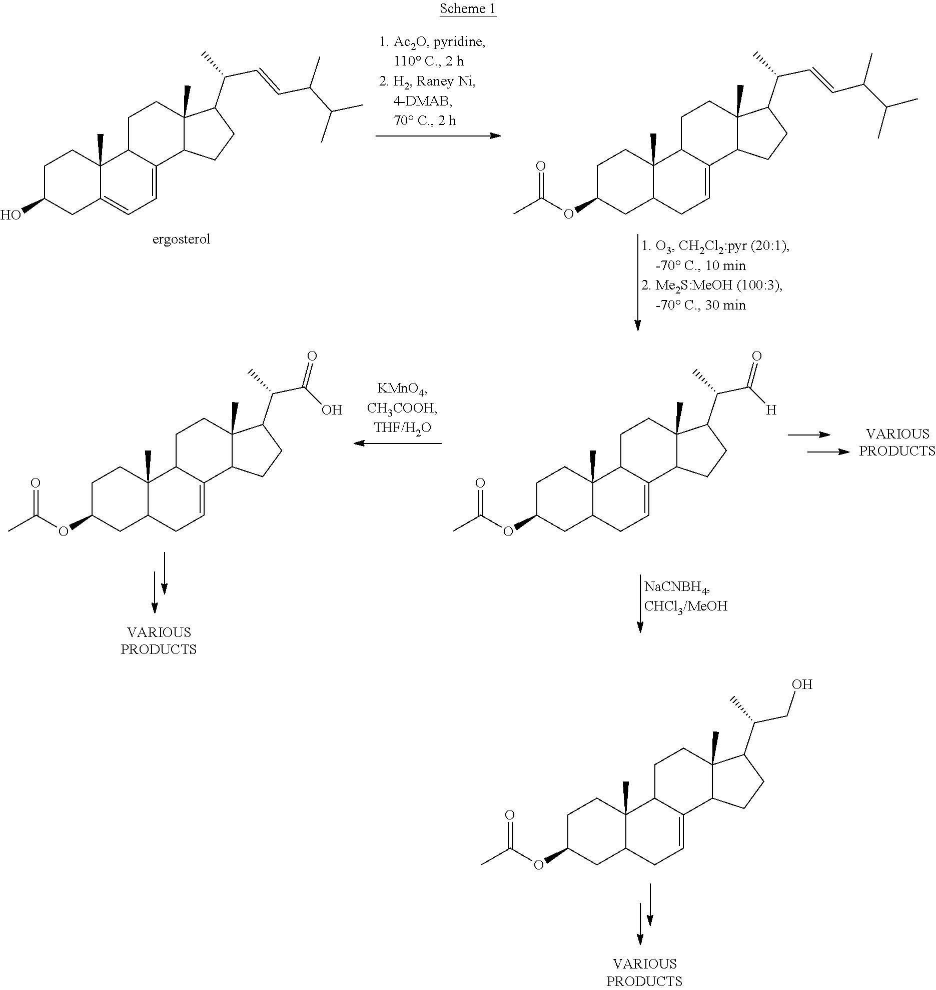 [SMILES: CC(C)C(C)/C=C/[C@@H](C)C1CCC2C3=CC=C4C[C@@H](O)CC[C@]4(C)C3CC[C@@]21C ; CC(=O)O[C@H]1CC[C@@]2(C)C(CC=C3C2CC[C@@]2(C)C3CCC2[C@H](C)/C=C/C(C)C(C)C)C1 ; CC(=O)O[C@H]1CC[C@@]2(C)C(CC=C3C2CC[C@@]2(C)C3CCC2[C@H](C)C(=O)O)C1 ; CC(=O)O[C@H]1CC[C@@]2(C)C(CC=C3C2CC[C@@]2(C)C3CCC2[C@H](C)C=O)C1 ; CC(=O)O[C@H]1CC[C@@]2(C)C(CC=C3C2CC[C@@]2(C)C3CCC2[C@H](C)CO)C1]

The compounds of Examples 1, 9 to 11, 16, 21, 25, 28, 29 were prepared as described in C. Müller et al., Eur. J. Med. Chem., 2017, 140, 305-320.

The compounds of Example 2 to 8, 12, 14, 15, 17 to 20, 22 to 24, 26, 27 were prepared as described in Sandra Hemmers, “Seitenkettenfunktionalisierte Steroide als Inhibitoren der Ergosterol-und Cholesterolbiosynthese”, PHD thesis, Munich, 2012 (DOI: 10.5282/edoc. 15485).

The compounds of Example 13 were prepared as described in D. Renard et al., Bioorg. Med. Chem., 2009, 17, 8123-8137.

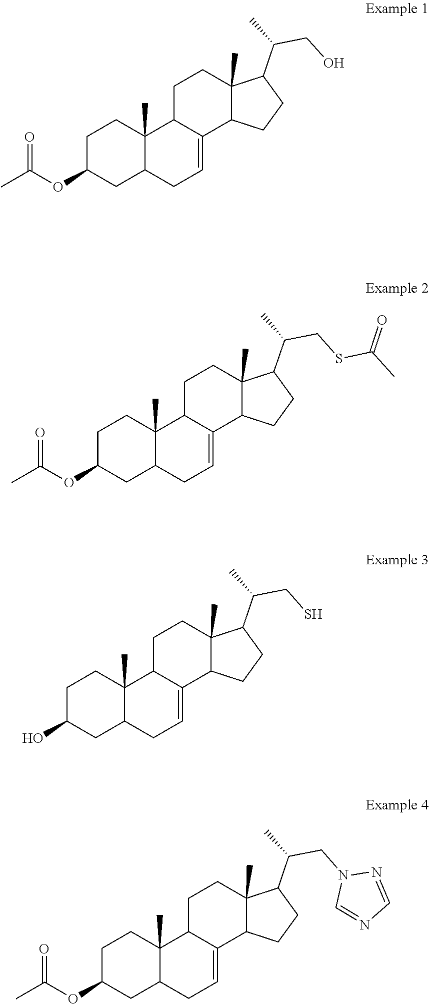

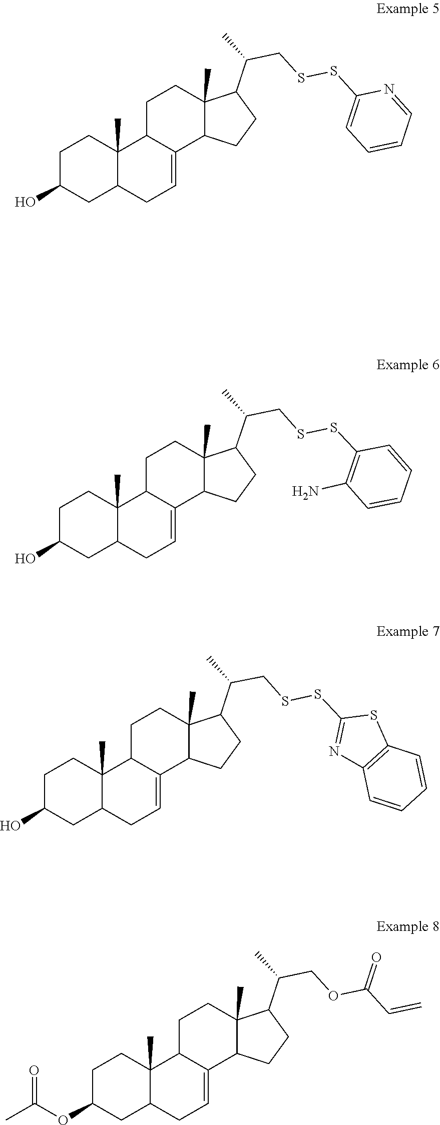 [SMILES: C[C@H](CSSc1ccccn1)C1CCC2C3=CCC4C[C@@H](O)CC[C@]4(C)C3CC[C@@]21C ; C[C@H](CSSc1ccccc1N)C1CCC2C3=CCC4C[C@@H](O)CC[C@]4(C)C3CC[C@@]21C ; C[C@H](CSSc1nc2ccccc2s1)C1CCC2C3=CCC4C[C@@H](O)CC[C@]4(C)C3CC[C@@]21C ; C=CC(=O)OC[C@@H](C)C1CCC2C3=CCC4C[C@@H](OC(C)=O)CC[C@]4(C)C3CC[C@@]21C]

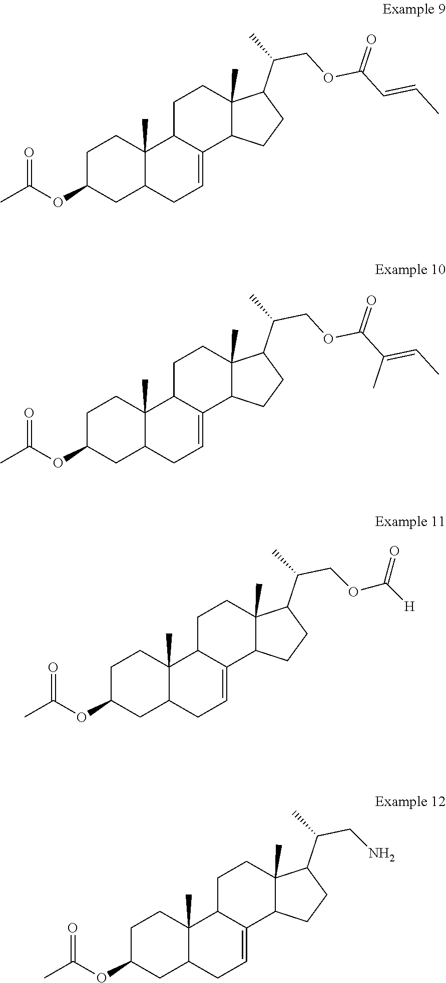

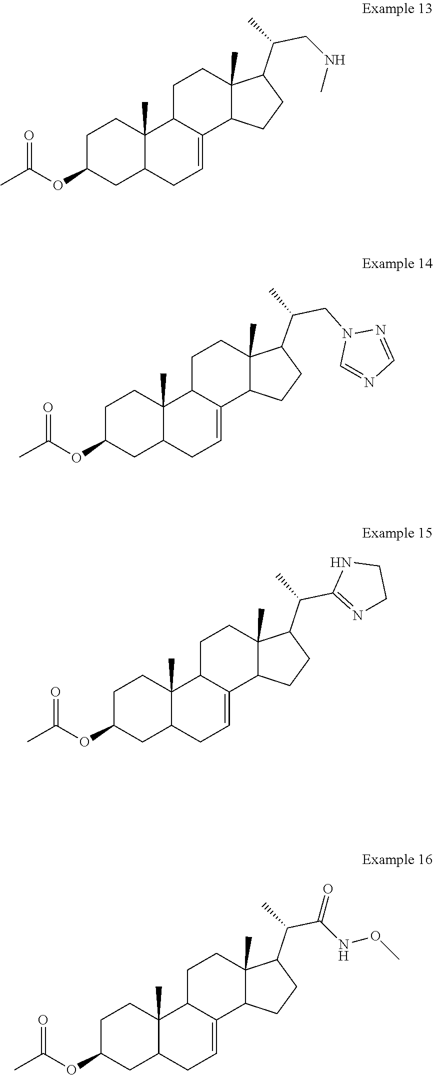 [SMILES: CNC[C@@H](C)C1CCC2C3=CCC4C[C@@H](OC(C)=O)CC[C@]4(C)C3CC[C@@]21C ; CC(=O)O[C@H]1CC[C@@]2(C)C(CC=C3C2CC[C@@]2(C)C3CCC2[C@H](C)Cn2cncn2)C1 ; CC(=O)O[C@H]1CC[C@@]2(C)C(CC=C3C2CC[C@@]2(C)C3CCC2[C@H](C)C2=NCCN2)C1 ; CONC(=O)[C@@H](C)C1CCC2C3=CCC4C[C@@H](OC(C)=O)CC[C@]4(C)C3CC[C@@]21C]

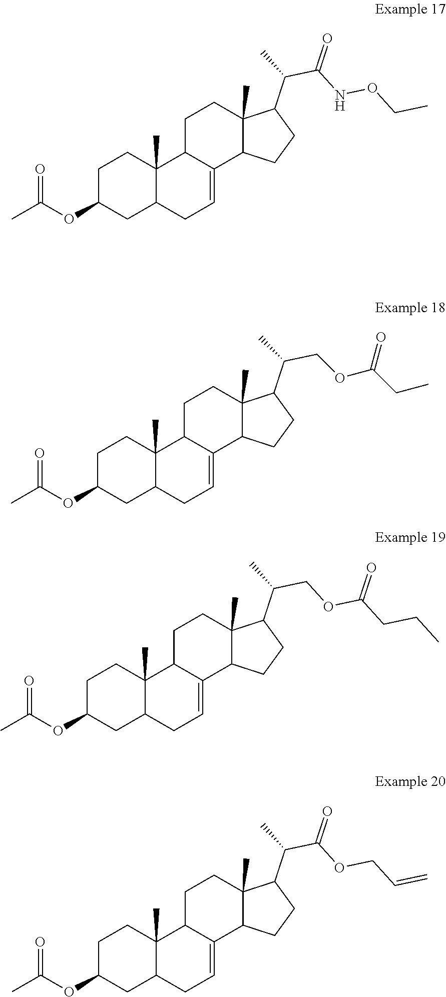

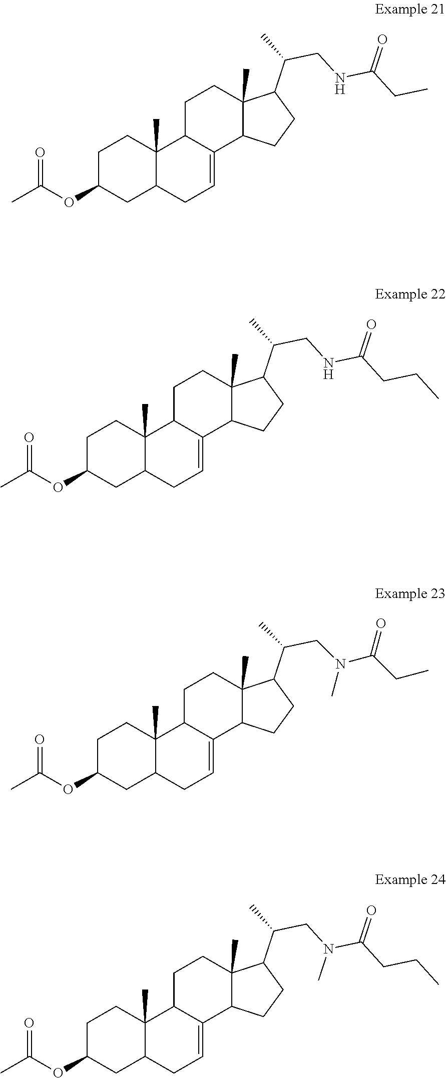

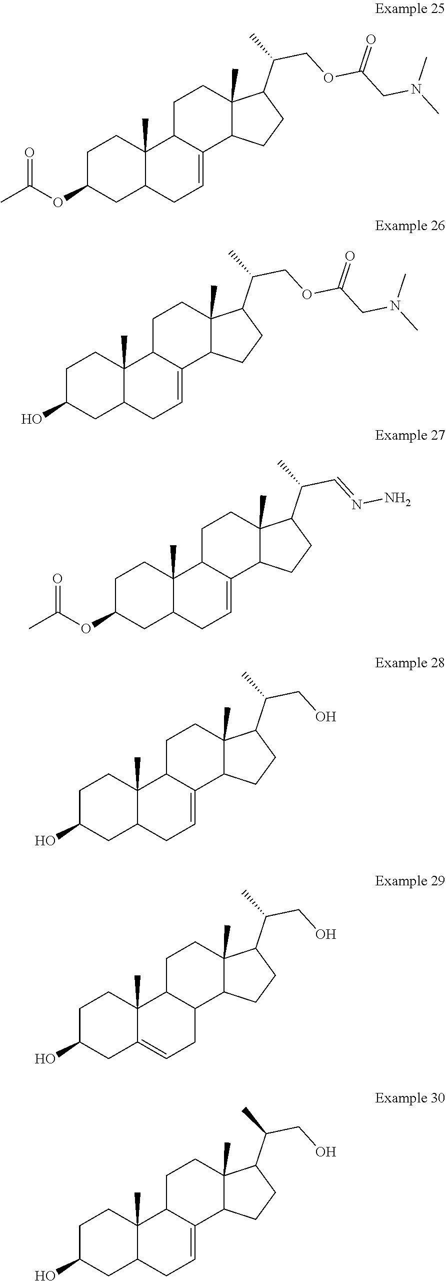

### (3S,20R)-20-(Hydroxymethyl)-pregn-7-en-3-yl Acetate (MiH-7)

A flame dried Schlenk flask was charged with (3S,20S)-20-formylpregn-7-en-3-yl acetate (151 mg, 0.405 mmol) and p-toluenesulfonic acid (7.8 mg, 0.041 mmol). After dissolving in dry toluene (1 mL) under nitrogen atmosphere, piperidine (0.642 mL, 6.5 mmol) was added and the solution was stirred for 3 hours at 70° C. After cooling to room temperature, the reaction mixture was diluted with ethyl acetate (EtOAc) and washed three times with 0.1 M HCl. The organic layer was dried using a hydrophobic filter paper and the solvent was removed in vacuo. The crude product was purified by flash column chromatography (FCC)(hexane/EtOAc 90:10), yielding 95 mg (63%) of a white solid that was directly subjected to reduction. Therefore, the previously obtained aldehyde (184 mg, 0.494 mmol) was dissolved in a flame dried Schlenk flask under nitrogen atmosphere in a 3:2 mixture of dry CHCl₃/MeOH. After the addition of sodium borohydride (18.9 mg, 0.5 mmol) the mixture was stirred for 2 hours at room temperature. The reaction was quenched using saturated NH₄Cl solution and the aqueous layer was extracted with CHCl₃ (3×3 mL). The combined organic layers were dried using a hydrophobic filter paper und the solvent was removed in vacuo. After purification using FCC (hexane/EtOAc 90:10→80:20) (3S,20R)-20-(hydroxymethyl)-pregn-7-en-3-yl acetate was obtained as a colourless solid (65 mg, 0.17 mmol, 35%).

¹H NMR (500 MHZ, CDCl₃) δ 5.16 (m, 1H, H-7), 4.69 (tt, ³J_{H3-H2,H4}=11.3, 4.6 Hz, 1H, H-3), 3.73 (dd, ³J_{H22-H22}=10.6, ³J_{H22-H20}=3.6 Hz, 1H, H-22), 3.49 (dd, ³J_{H22-H22}=10.6, ³J_{H22-H20}=6.9 Hz, 1H, H-22), 2.02 (s, 3H, CH₃CO), 1.93-1.30 (m, 20H), 1.25 (tdz, J=12.9, 4.2 Hz, 1H), 1.17-1.09 (m, 1H, H-17), 0.96 (d, ³J_{H21-H20}=6.7 Hz, 3H, H-21), 0.81 (s, 3H, H-19), 0.55 (s, 3H, H-18). ¹³C NMR (126 MHZ, CDCl₃) δ 170.8 (CH₃CO), 139.3 (C-8), 117.8 (C-7), 73.6 (C-3), 67.1 (C-22), 55.0 (C-14), 52.6 (C—C20), 49.3 (C-9), 43.3 (C-13), 40.2 (C-5), 39.1 (C-12), 38.4 (C-17), 37.0 (C-1), 34.34 (C-10), 33.9 (C-11), 29.7 (C-6), 27.6 (C-2 or C-4), 27.4 (C-2 or C-4), 22.9 (C-15 or C-16), 21.6 (CH₃CO), 21.6 (C-15 or C-16), 16.8 (C-21), 13.1 (C-19), 12.4 (C-18). HR-MS (EI): calcd. for C₂₄H₃₈O₃: 374.2821, found 374.2816.

### (3S,20R)-20-(Hydroxymethyl)-pregn-7-en-3-ol (MiH-8)

To a stirred solution of (3S,20R)-20-(hydroxymethyl)-pregn-7-en-3-yl acetate (65 mg, 0,174 mmol) in MeOH was added a solution of K₂CO₃ (288 mg, 174 mmol) in water (2.35 mL). The mixture was heated to reflux for 2 hours, then cooled to room temperature and concentrated under reduced pressure. The precipitate was redissolved in EtOAc and the organic layer was washed twice each with saturated KHCO₃ solution, water and brine. After filtering the organic layer through a hydrophobic filter paper to remove water, the solvent was removed in vacuo. After purification through FCC (hexane/EtOAc 70:30)(3S,20R)-20-(hydroxymethyl)-pregn-7-en-3-ol was obtained as colourless to greyish solid (28 mg, 0,084 mmol, 48%).

¹H NMR (400 MHZ, DMSO-d₆) δ 5.14-5.08 (m, 1H, H-7), 4.45 (s, 1H, OH), 4.27 (s, 1H, OH), 3.54 (d, J=10.3 Hz, 1H, H-22), 3.33 (s, 6H, H-3 underneath), 3.10 (t, J=8.9 Hz, 1H, H-22), 1.91-0.93 (m, 27H), 0.85 (d, J=6.6 Hz, 3H, H-21), 0.72 (s, 3H, H-19), 0.49 (s, 3H, H-18). ¹³C NMR (101 MHZ, DMSO-d₆) δ 139.1 (C-8), 117.2 (C-7), 68.90 (C-3), 64.9 (C-22), 54.4 (C-14), 52.4 (C-17), 48.9 (C-9), 42.8 (C-13), 39.5 (C-5, underneath DMSO), 38.4 (C-12), 38.1 (C-20), 37.9 (CH₂), 36.7 (CH₂), 33.8 (C-10), 31.3 (C-1), 29.3 (CH₂), 26.6 (CH₂), 22.4 (CH₂), 21.0 (CH₂), 16.7 (C-21), 12.9 (C-19), 12.1 (C-18). HR-MS (EI): calcd. for C₂₂H₃₆O₂ 332.2715, found 332.2711.

### Example 31

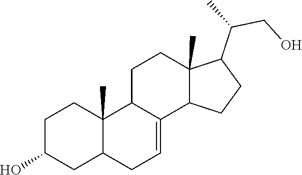 [SMILES: C[C@H](CO)C1CCC2C3=CCC4C[C@H](O)CC[C@]4(C)C3CC[C@@]21C]

### (3S,20S)-20-(Hydroxymethyl)-pregn-7-en-3-ol (MiH-10)

Lithium aluminium hydride (56.7 mg, 1.49 mmol) was dispersed in dry THF in a flame dried Schlenk flask under nitrogen atmosphere. The mixture was cooled to 0° C. in an ice bath and a solution of (3S,20S)-20-formylpregn-7-en-3-yl acetate (105 mg, 0.282 mmol) in THF (1 mL) was added dropwise. Stirring was continued overnight while the mixture was warmed to room temperature. The reaction was quenched with water and the mixture was acidified with 1 M HCl. The aqueous layer was extracted with EtOAc (3×3 mL) and the combined organic layers were filtered through a hydrophobic filter paper to remove water. After removing the solvent under reduced pressure the crude product was subjected to FCC (hexane/EtOAc 1:1). The diol was obtained as colourless solid (63 mg, 0.19 mmol, 67%).

¹H NMR (400 MHZ, CDCl₃) δ 5.19-5.14 (m, 1H, H-7), 3.64 (dd, J=10.8, J=3.5 Hz, 1H, H-22), 3.58 (dt, J=10.6, 4.5 Hz, 1H, H-3), 3.39 (dd, J=10.3, J=7.0 Hz, 1H, H-22), 2.05-1.98 (m, 1H, H-6), 1.93-1.17 (m, 22H), 1.06 (d, J=6.6 Hz, 3H, H-21), 0.80 (s, 3H, H-19), 0.56 (s, 3H, H-18). ¹³C NMR (101 MHZ, CDCl₃) δ 139.5 (C-8), 117.8 (C-7), 71.2 (C-3), 68.1 (C-22), 54.9 (C-14), 52.5 (C-17), 49.6 (C-5), 43.6 (C-13), 40.4 (C-9), 39.6 (C-6), 39.3 (C-20), 38.1 (C-2), 37.3 (C-1), 34.4 (C-10), 31.6 (CH₂), 29.8 (CH₂), 27.6 (CH₂), 23.2 (CH₂), 21.7 (CH₂), 17.0 (C-21), 13.2 (C-19), 12.1 (C-18). HR-MS (EI): calcd. for C₂₂H₃₆O₂: 332.2715, found 332.2710.

### (3R,20S)-20-[(4-Nitrobenzoyloxy)methyl]-pregn-7-en-3-yl 4-nitrobenzoate

(3S,20S)-20-(Hydroxymethyl)-pregn-7-en-3-ol (54 mg, 0.16 mmol), 4-nitrobenzoic acid (204 mg, 1.22 mmol) and triphenylphosphine (319 mg, 1.22 mmol) were dissolved in dry THF (1 mL) under nitrogen atmosphere, followed by cooling the mixture to 0° C. in an ice bath. Diisopropylazodicarboxylate (DIAD)(0.113 mL, 0.568 mmol) was added dropwise. The reaction was warmed to room temperature and stirred overnight, then quenched by the addition of saturated NH₄Cl (2 mL). After extracting the aqueous layer with DCM (3×3 mL), the combined organic extracts were filtered through a hydrophobic filter paper to remove water. Solvent was removed under reduced pressure and the crude product was subjected to FCC (hexane/EtOAc 80:20). (3R,20S)-20-[(4-Nitrobenzoyloxy)methyl]-pregn-7-en-3-yl 4-nitrobenzoate was obtained as a colourless solid (76 mg, 0.12 mmol, 74%).

¹H NMR (500 MHZ, CDCl₃) δ 8.32-8.26 (m, 4H, aromatic), 8.23-8.18 (m, 4H, aromatic), 5.35 (t, J=2.8 Hz, 1H, H-3), 5.24 (m, 1H, H-7), 4.41 (dd, J=10.7, 3.5 Hz, 1H, H-22), 4.10 (dd, J=10.7, 7.6 Hz, 1H, H-22), 2.08 (dt, J=12.5, 3.7 Hz, 1H, H-6), 2.00-1.24 (m, 22H), 1.15 (d, J=6.6 Hz, 3H, H-21), 0.87 (s, 3H, H-19), 0.63 (s, 3H, H-18). ¹³C NMR (126 MHZ, CDCl₃) δ 165.0 (COO), 164.1 (COO), 150.7 (aromatic), 150.6 (aromatic), 139.3 (C-8), 136.6 (aromatic), 136.1 (aromatic), 130.8 (aromatic), 123.7 (aromatic), 123.7 (aromatic), 118.1 (C-7), 72.1 (C-3), 71.0 (C-22), 55.0 (C-14), 53.0 (C-17), 49.7 (C-9), 43.9 (C-13), 39.5 (C-6), 36.6 (C-20), 36.1 (C-5), 34.7 (C-10), 33.2 (CH₂), 32.6 (C-4), 29.5 (CH₂), 27.7 (CH₂), 26.2 (C-2), 23.2 (CH₂), 21.4 (C-1), 17.6 (C-21), 12.4 (C-19), 12.1 (C-18). HR-MS (EI): calcd. for C₃₆H₄₂N₂O₈ 630.2941, found 630.2932.

### (3R,20S)-20-(Hydroxymethyl)-pregn-7-en-3-ol (MiH-12)

(3R,20S)-20-[(4-Nitrobenzoyl)methyl]-pregn-7-en-3-yl 4-nitrobenzoate (80 mg, 0.13 mmol) was dissolved in THF (2 mL) and cooled to 0° C. in an ice bath, 0.38 mL of a 2 M NaOH solution were added slowly, the mixture was warmed to room temperature and stirred overnight. The reaction mixture was diluted with DCM (5 mL) and the organic layer was washed three times each with water, 1 M HCl and brine. After filtration through a hydrophobic filter paper to remove water, the solvent was removed under reduced pressure. The crude product was subjected to FCC (hexanes/EtOAc 70:30) to obtain (3R,20S)-20-(hydroxymethyl)-pregn-7-en-3-ol as a colourless to light yellow solid (36 mg, 0.11 mmol, 85%).

¹H NMR (500 MHZ, DMSO) δ 5.13 (m, 1H, H-7), 4.28 (dd, ³J_{H22-OH}=5.7, 4.8 Hz, 1H, OH), 4.20 (d, ³J_{H3-OH}=3.2 Hz, 1H, OH), 3.83 (q, ³J_{H3-H2}, J_{H3-H4}=2.9 Hz, 1H, H-3), 3.42 (s, 1H, H-22), 3.07 (ddd, ³J_{H17-H20}=10.3, ³J_{H20-H22}=7.1, ³J_{H20-H21}=5.8 Hz, 1H, H-22), 1.99-1.09 (m, 21H), 0.95 (d, ³J_{H21-H20}=6.5 Hz, 3H, H-21), 0.71 (s, 3H, H-19), 0.50 (s, 3H, H-18). 13° C. NMR (126 MHZ, DMSO) δ 139.1 (C-8), 117.5 (C-7), 65.7 (C-22), 64.0 (C-3), 54.3 (C-14), 52.2 (C-17), 49.1 (C-5), 43.1 (C-13), 40.1 (C-20), 38.9 (C-6), 35.3 (C-2), 34.3 (C-10), 34.2 (C-9), 31.7 (C-1), 29.2 (C-11), 28.5 (C-12), 27.1 (C-4), 22.7 (CH₂), 20.8 (CH₂), 17.2 (C-20), 12.1 (C-19), 11.9 (C-18). HR-MS (EI): calcd. for C₂₂H₃₆O₂ 332.2715, found 332.2711.

### Example 32

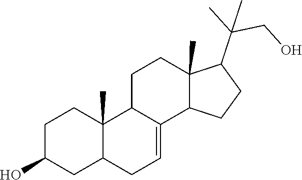 [SMILES: CC(C)(CO)C1CCC2C3=CCC4C[C@@H](O)CC[C@]4(C)C3CC[C@@]21C]

### (3S)-20-Formyl-20-methylpregn-7-en-3-yl Acetate (MiH-6)

(3S,20S)-20-Formylpregn-7-en-3-yl acetate (250 mg, 0.671 mmol) and KOtBu (753 mg, 6.71 mmol) were dissolved in dry DCM (10 mL) at room temperature under nitrogen atmosphere. When a white precipitate had formed, methyl iodide (1.25 mL, 20.1 mmol) was added dropwise. The mixture was stirred for three hours and then quenched by adding saturated NH₄Cl solution (5 mL). The aqueous layer was extracted three times with DCM and the combined organic extracts were filtered through a hydrophobic filter paper to remove water. Solvent was removed under reduced pressure and the crude product was subjected to FCC (hexane/EtOAc 95:5). (3S)-20-Formyl-20-methylpregn-7-en-3-yl acetate was obtained as a colourless solid (155 mg, 0.401 mmol, 59%).

¹H NMR (400 MHZ, CDCl₃) δ 9.63 (s, 1H, CHO), 5.18-5.14 (m, 1H, H-7), 3.34 (s, 3H, H₃CCOO), 3.12 (tt, J=11.1, 4.4 Hz, 1H, H-3), 1.93-1.53 (m, 18H), 1.50-1.13 (m, 10H), 1.11 (s, 3H, H-21 or H-21′), 1.09 (s, 3H, H-21 or H-21′), 1.03 (td, J=13.5, 3.4 Hz, 1H, H-1), 0.76 (s, 3H, H-19), 0.55 (s, 3H, H-18). ¹³C NMR (101 MHZ, CDCl₃) δ 206.80 (CHO), 170.8 (H₃CCOO), 138.8 (C-8), 118.5 (C-7), 79.7 (C-3), 57.5 (C-17), 55.7 (H₃CCOO), 55.0 (C-14), 49.5 (C-9), 48.9 (C-20), 44.2 (C-13), 40.3 (C-5), 39.5 (C-12), 37.2 (C-1), 34.7 (C-10), 34.3 (CH₂), 29.9 (CH₂), 27.8 (CH₂), 23.0 (C-21 or C-21′), 22.6 (CH₂), 22.4 (CH₂), 21.5 (CH₂), 20.9 (C-21 or C-21′), 14.7 (C-18), 13.1 (C-19). HR-MS (EI): calcd. For C₂₅H₃₈O₃ 386.2821, found 386.2826.

### (3S)-20-Hydroxymethyl-20-methylpregn-7-en-3-yl Acetate (MiH-9)

(3S)-20-Formyl-20-methylpregn-7-en-3-yl acetate (128 mg, 0.331 mmol) was dissolved in a flame dried Schlenk flask under nitrogen atmosphere in a 3:2 mixture of dry CHCl₃/MeOH. After the addition of sodium borohydride (12.7 mg, 0.335 mmol) stirring was continued for 1 hour at room temperature. The reaction was quenched using saturated NH₄Cl solution (3 mL) and the aqueous layer was extracted with CHCl₃ (3×3 mL). The combined organic extracts were dried using a hydrophobic filter paper and the solvent was removed under reduced pressure. After purification using FCC (hexane/EtOAc 90:10→80:20)(3S)-20-hydroxymethyl-20-methylpregn-7-en-3-yl acetate was obtained as a colourless solid (87 mg, 0.22 mmol, 67%).

¹H NMR (500 MHZ, CDCl₃) δ 5.16 (dq, J=4.6, 2.3 Hz, 1H, H-7), 3.35 (s, 3H, CH₃CO), 3.40-3.31 (m, 2H, H-22), 3.12 (tt, J=11.2, 4.4 Hz, 1H, H-3), 2.06-2.01 (m, 1H, H-9), 1.91-1.00 (m, 20H), 0.99 (s, 3H, H-21 or H-211), 0.90 (s, 3H, H-21 or H-211), 0.78 (s, 3H, H-19), 0.62 (s, 3H, H-18). ¹³C NMR (126 MHZ, CDCl₃) δ 139.4 (H₃CCO), 118.1 (C-7), 79.7 (C-3), 77.4 (C-8), 72.8 (C-22), 55.7 (H₃CCO), 55.5 (C-5), 55.4 (C-17), 49.5 (C-14), 44.4 (C-13), 40.3 (C-9), 40.0, 39.0 (C-20), 37.2, 34.7 (C-10), 34.4, 29.9, 27.9, 23.6 (C-21 or C-211), 23.5 (C-21 or C-211), 22.8 (CH₂), 22.6 (CH₂), 21.6 (CH₂), 14.6 (C-19), 13.1 (C-18). HR-MS (EI): calcd. for C₂₂H₃₆O₂ 388,2977, found 388,2975.

### (3S)-20-Hydroxymethyl-20-methylpregn-7-en-3-ol (MiH-13)

To a stirred solution of (3S)-20-hydroxymethyl-20-methylpregn-7-en-3-yl acetate (80 mg, 0.21 mmol) in MeOH (25 mL) was added a solution of K₂CO₃ (342 mg, 2.06 mmol) in water (2.8 mL). The mixture was heated to reflux for 4 hours, then cooled to room temperature and concentrated under reduced pressure. The precipitate was re-dissolved in EtOAc and the organic layer was washed successively with saturated KHCO₃ solution (2×5 mL), water and brine. After filtering the organic layer through a hydrophobic filter paper to remove water, the solvent was removed in vacuo. After purification through FCC (hexane/EtOAc 70:30)(3S)-20-Hydroxymethyl-20-methylpregn-7-en-3-ol was obtained as colourless solid (24 mg, 0.069 mmol, 33%).

¹H NMR (500 MHZ, CDCl₃) δ 5.16 (m, 1H, H-7), 3.59 (tt, J=11.0, 4.5 Hz, 1H, H-3), 3.35 (q, J=10.6 Hz, 2H, H-22), 2.03 (ddd, J=12.3, 4.0, 2.6 Hz, 1H, H-9), 1.86-1.04 (m, 22H), 0.99 (s, 3H, H-21 or H-211), 0.90 (s, 3H, H-21 or H-211), 0.79 (s, 3H, H-19), 0.62 (s, 3H, H-18). ¹³C NMR (126 MHZ, CDCl₃) δ 139.4 (C-8), 118.1 (C-7), 72.8 (C-22), 71.2 (C-3), 55.5 (C-14), 55.4 (C-17), 49.5 (C-5), 44.4 (C-13), 40.4 (C-9), 40.0 (C-2), 39.0 (C-20), 38.1 (C-4), 37.3 (C-6), 34.3 (C-10), 31.6 (C-1), 29.8 (C-11), 23.6 (C-21 or C-211), 23.5 (C-21 or C-211), 22.8 (C-15), 22.6 (C-16), 21.6 (C-12), 14.6 (C-18), 13.2 (C-19). HR-MS (EI): calcd. for C₂₃H₃₈O₂ 346.2872, found 346.2876.

### Example 33

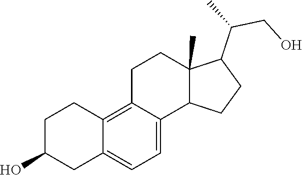 [SMILES: C[C@H](CO)C1CCC2c3ccc4c(c3CC[C@@]21C)CC[C@H](O)C4]

### (3S,20S)-20-(3,4-Dimethylpent-1-en-yl)-pregn-5,7,9-trien-3-ol (MiH-16)

To a solution of eosin Y (5015 mg, 7.25 mmol) in 95% EtOH (150 mL) was added concentrated H₂SO₄ (0.402 mL, 7.25 mmol). The forming white precipitate was removed from the solution through vacuum filtration and nitrogen was passed through for 30 minutes. Ergosterol (5000 mg, 12.6 mmol) and 75 mL THF were added in the dark. The 500 mL round bottom flask containing the reaction mixture and a stirring bead was placed in a box equipped with a LED band fixed onto a highly polished interior. The reaction mixture was vigorously stirred while being exposed to blue LED light for 38 hours. The colourless precipitate which had formed was filtered and washed using cold EtOH, then dried in vacuo to give bis ergostatrienol as a colourless to light yellow solid (3510 mg, 4.44 mmol, 70%), which was used for the next step without further purification. To a flame dried 500 mL round bottom flask, equipped with a boiling bead and a reflux condenser was added the above mentioned solid (930 mg, 1.18 mmol), suspended in dry toluene under nitrogen atmosphere. The mixture was heated to reflux and when all solids had dissolved, N,O-bis(trimethylsilyl) acetamide (0.632 mL, 2.59 mmol) was added dropwise. Stirring was continued for 5.5 hours, then the reaction mixture was cooled to room temperature and the solvent was removed under reduced pressure. To the precipitate were added 30 mL acetone and heated to reflux for 5 minutes. A white precipitate formed upon cooling, which was collected by filtration and dried in vacuo to give bis-TMS-bis ergostatrienol as crude product (816 mg, 0.872 mmol, 74%). After that, the crude product (753 mg, 0.805 mmol) was dissolved in diethyleneglycol ethyl ether (40 mL) under nitrogen atmosphere in a round bottom flask equipped with a reflux condenser and a boiling bead. The mixture was heated to reflux for 10 minutes, then rapidly cooled down to 100° C. and poured into a stirred mixture of ice and water containing a few crystals of p-toluenesulfonic acid. After 24 h at −20° C., the white precipitate was collected by filtration and washed with cold water. Recrystallisation from MeOH gave neoergosterol as white powder (401 mg, 1.05 mmol, 65%).

¹H NMR (400 MHZ, CDCl₃) δ 6.90 (d, J=7.9 Hz, 1H, H-6), 6.85 (d, J=7.8 Hz, 1H, H-7), 5.30-5.18 (m, 2H, H-22, H-23), 4.17-4.07 (m, 1H, H-3), 3.10-3.02 (m, 1H, CH₂), 2.82-2.72 (m, 2H, CH₂), 2.70-2.59 (m, 4H, CH₂), 2.27-2.19 (m, 1H, CH₂), 2.16-2.00 (m, 4H, CH₂, H-20), 1.97-1.77 (m, 4H, CH₂, H-24), 1.73-1.32 (m, 9H, CH₂, H-17, H-25), 1.09 (d, J=6.6 Hz, 3H, H-21), 0.97-0.90 (m, 3H, H-24¹), 0.84 (ddq, J=7.1, 4.5, 2.2, 1.8 Hz, 6H, H-26, H-27), 0.60 (s, 3H, H-18). ¹³C NMR (101 MHZ, CDCl₃) δ 138.1 (C-9), 135.7 (C-22), 134.2 (C-8), 133.6 (C-10), 132.2 (C-23), 131.3 (C-5), 127.0 (C-6), 124.0 (C-7), 67.3 (C-3), 55.2 (C-17), 52.0 (C-14), 43.0 (C-24), 41.9 (C-13), 40.7 (C-20), 39.1 (CH₂), 37.0 (CH₂), 33.3 (C-25), 31.8 (CH₂), 29.3 (CH₂), 25.3 (CH₂), 24.8 (CH₂), 24.3 (CH₂), 21.2 (C-21), 20.1 (C-25 or C-26), 19.8 (C-25 or C-26), 17.8 (C-24¹), 11.4 (C-18). HR-MS (EI): calcd. for C₂₇H₄₀O₁ 380.3079, found 380.3079.

### (3S,20S)-20-(Hydroxymethyl)-pregn-5,7,9-trien-3-ol (MiH-17, Example 33)

To a solution of neoergosterol (180 mg, 0.473 mmol) in DCM (23 mL) in a 100 mL gas washing bottle equipped with a stirring bead and glass frit were added pyridine (0.191 mL, 2.36 mmol) and few crystals of sudan III. While passing through nitrogen, the solution was cooled to −50° C. After that, ozone was introduced through the glass frit (60 L/h, 60 W) until the colour of the solution had changed to light yellow. Nitrogen was introduced for 10 minutes, then methanol (3.6 mL) was added, followed by the addition of sodium borohydride (179 mg, 4.73 mmol) in small portions. Stirring was continued while slowly warming to room temperature. The reaction was quenched by the addition of 0.1 M HCl (10 mL) and the aqueous layer was extracted with DCM (3×10 mL). The combined organic layers were washed with 0.1 M HCl (3×15 mL) and then filtered through a hydrophobic filter paper to remove water. The solvent was removed under reduced pressure. The crude product was subjected to FCC (hexanes/EtOAc 70:30→1:1) to give (3S,20S)-20-(hydroxymethyl)-pregn-5,7,9-trien-3-ol as colourless solid (80 mg, 0.25 mmol, 53%).

¹H NMR (400 MHZ, DMSO) δ 6.79 (d, J=7.8 Hz, 1H, H-6), 6.72 (d, J=7.7 Hz, 1H, H-7), 4.72 (d, J=3.8 Hz, 1H, HO—C-3), 4.30 (t, J=5.3 Hz, 1H, CH₂—OH), 3.82 (tt, J=8.5, 3.7 Hz, 1H, H-3), 3.48-3.41 (m, 1H, H-22), 3.13 (dt, J=10.2, 6.3 Hz, 1H, H-22), 2.85 (dd, J=16.0, 4.7 Hz, 1H, H-4)), 2.69-2.47 (m, 13H), 2.16 (dt, J=12.7, 4.7 Hz, 1H, H-12 und CH₂), 2.06-1.89 (m, 3H), 1.66-1.54 (m, 2H, H-12 und CH₂), 1.51-1.29 (m, 4H, H-17, H-20 und CH₂), 1.02 (d, J=6.4 Hz, 3H, H-21), 0.51 (s, 3H, H-18). ¹³C NMR (101 MHZ, DMSO) δ 136.7 (C-9), 133.3 (C-8), 133.1 (C-10), 132.0 (C-5), 126.4 (C-6), 123.1 (C-7), 65.6 (C-22), 65.1 (C-3), 51.2 (C-17), 51.0 (C-14), 41.4 (C-13), 39.0 (C-20), 38.8 (C-4), 36.3 (C-12), 31.6 (CH₂), 28.0 (CH₂), 24.4 (CH₂), 24.0 (2×CH₂), 17.0 (C-21), 11.0 (C-18). HR-MS (EI): calcd. for C₂₁H₃₀O₂ 314.2246, found 314.2237.

### Example 34

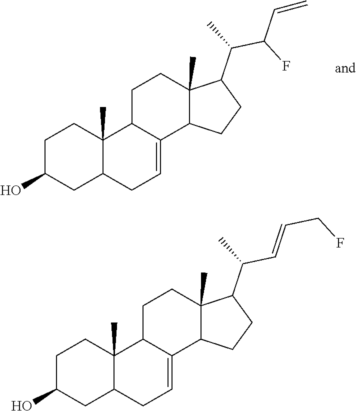 [SMILES: C=CC(F)[C@@H](C)C1CCC2C3=CCC4C[C@@H](O)CC[C@]4(C)C3CC[C@@]21C ; C[C@H](/C=C/CF)C1CCC2C3=CCC4C[C@@H](O)CC[C@]4(C)C3CC[C@@]21C]

### (3S,20R)-20-(2-Formyloxyethyl)-pregn-7-en-3-yl Acetate (MiH-58)

In a flame dried roundbottom flask equipped with a stirring bead, (3S,20R)-20-(hydroxyethyl)-pregn-7-en-3-yl acetate (60 mg, 0.15 mmol)(prepared as described in Sandra Hemmers, “Seitenkettenfunktionalisierte Steroide als Inhibitoren der Ergosterol-und Cholesterolbiosynthese”, PhD thesis, LMU Munich, 2012, DOI: 10.5282/edoc. 15485), and DMAP (9.4 mg, 0.077 mmol) were dissolved in dry DCM (4 mL) under nitrogen atmosphere. To the solution were added dropwise a solution of 1-(3-dimethylaminopropyl)-3-ethylcarbodiimide hydrochloride (136 mg, 0.695 mmol) in dry DCM (3 mL) and formic acid (0.029 mL, 0.70 mmol). The mixture was stirred for 6 days at room temperature, then quenched with 1 M HCl (4 mL). The aqueous layer was extracted with DCM (3×3 mL) and the combined organic extracts were washed with water and brine. Before removing the solvent in vacuo, water was removed through filtration through a hydrophobic filter paper. Purification by FCC (hexanes/EtOAc 90:10) afforded (3S,20R)-20-(hydroxyethyl)-pregn-7-en-3-yl acetate as a colourless solid (44 mg, 0.11 mmol, 68%).

¹H NMR (400 MHZ, CDCl₃) δ 8.05 (s, 1H, CHO), 5.15 (dd, J=5.2, 2.5 Hz, 1H, H-7), 4.69 (tt, J=11.2, 4.6 Hz, 1H, H-3), 4.24 (ddd, J=10.7, 7.8, 5.0 Hz, 1H, H-23), 4.16 (dt, J=10.9, 7.6 Hz, 1H, H-23), 2.02 (s, 3H, H₃CCOO), 2.00 (t, J=3.6 Hz, 1H, H-12), 1.97-1.09 (m, 25H), 0.98 (d, J=6.7 Hz, 3H, H-21), 0.81 (s, 3H, H-18), 0.54 (s, 3H, H-19). ¹³C NMR (101 MHZ, CDCl₃) δ 170.83 (H₃CCOO), 161.38 (CHO), 139.38 (C-8), 117.70 (C-7), 73.59 (C-3), 62.40 (C-23), 56.13 (C-17), 55.08 (C-14), 49.35 (C-9), 43.58 (C-13), 40.17 (C-5), 39.60 (C-12), 36.97 (C-1), 34.55 (C-22), 34.34 (C-10), 33.93 (C-4), 33.62 (C-20), 29.66 (C-6), 28.07 (C-16), 27.63 (C-2), 23.04 (C-15), 21.61 (H₃CCOO), 21.58 (C-11), 18.93 (C-21), 13.08 (C-19), 11.95 (C-18). HR-MS (EI): calcd. for C26H40O4 416.2927, found 416.2919.

### Example 35

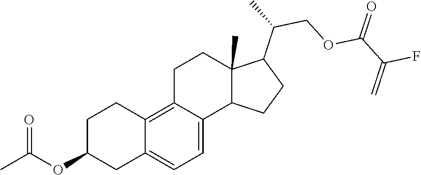 [SMILES: C=C(F)C(=O)OC[C@@H](C)C1CCC2c3ccc4c(c3CC[C@@]21C)CC[C@H](OC(C)=O)C4]

### (3S,20S)-20-(3,4-Dimethylpent-1-en-yl)-pregn-5,7,9-trien-3-yl Acetate (MiH-31)

Neoergosterol (1976 mg, 5.19 mmol) was added to a round bottom flask charged with acetic anhydride (41 mL) and pyridine (0.83 mL, 10 mmol). The mixture was heated to 120° C. for two hours, then cooled to room temperature overnight. A white precipitate was formed which was filtered and washed with cold MeOH. After drying in vacuo, neoergosteryl acetate was obtained as a colourless solid (1452 mg, 3.44 mmol, 66%).

¹H NMR (400 MHZ, CDCl₃) δ 6.87 (q, J=7.9 Hz, 2H, H-6, H-7), 5.31-5.20 (m, 2H, H-22, H-23), 5.15 (tdd, J=8.1, 6.2, 4.2 Hz, 1H, H-3), 3.09 (dd, J=16.3, 5.0 Hz, 1H, H-4), 2.87 (dd, J=16.2, 7.7 Hz, 1H, H-4), 2.67 (d, J=6.1 Hz, 5H, H-14 und CH₂), 2.23 (ddd, J=12.9, 6.7, 2.5 Hz, 1H, CH₂), 2.10 (ddd, J=15.9, 5.4, 4.0 Hz, 2H, H-20 und CH₂), 2.06 (s, 3H, H₃CCOO), 1.98-1.84 (m, 3H, H-24 und CH₂), 1.73-1.63 (m, 1H, CH₂), 1.54-1.13 (m, 5H, H-17 und CH₂), 1.09 (d, J=6.6 Hz, 3H, H-21), 0.94 (d, J=6.9 Hz, 3H, H-24¹), 0.89-0.82 (m, 6H, H-27, H-26), 0.61 (s, 3H, H-18). ¹³C NMR (101 MHz, CDCl₃) δ 171.0 (H₃CCOO), 138.1 (C-9), 135.7 (C-22), 134.2 (C-8), 133.2 (C-10), 132.2 (C-23), 130.8 (C-5), 126.7 (C-6), 124.0 (C-7), 70.0 (C-3), 55.2 (C-17), 51.9 (C-14), 43.0 (C-24), 41.9 (C-13), 40.7 (C-20), 37.0 (C-12), 35.2 (CH₂), 33.3 (C-25), 29.3 (CH₂), 28.2 (CH₂), 25.2 (CH₂), 24.3 (CH₂), 24.3 (CH₂), 21.6 (H₃CCOO), 21.2 (C-21), 20.1 (C-26 or C-27), 19.8 (C-26 or C-27), 17.8 (C-24¹), 11.5 (C-18). HR-MS (EI): calcd. for C₂₉H₄₂O₂ 422.3185, found 362.3080 (—H₃CCOOH).

### (3S,20S)-20-Formylpregn-5,7,9-trien-3-yl Acetate (MiH-32)

To a solution of neoergosteryl acetate (1700 mg, 4.02 mmol) in a gas washing bottle equipped with a stirring bead and a glass frit in dry DCM were added pyridine (2.0 mL, 25 mmol) and a few crystals of sudan III. The solution was cooled to −78° C. using a dry ice-acetone bath while passing nitrogen through the solution using the glass frit. Afterwards, ozone was introduced through the glass frit (60 L/h, 60 W) until the colour of the solution changed from light pink to light yellow. Again, nitrogen was passed through the solution, followed after 10 minutes by the addition of dimethylsulfide (0.59 mL, 8.0 mmol) dissolved in MeOH (20 mL). The stirring was continued for further 30 minutes at −78° C., then the reaction mixture was warmed to room temperature. After removing the solvent under reduced pressure, the oily residue was dissolved in ethyl acetate (40 mL) and washed with 5% KHSO₄ solution (3×40 mL). The organic layer was filtered through a hydrophobic filter paper to remove water, and the solvent was removed in vacuo. The crude product was subjected to FCC (hexane/EtOAc 90:10) to give (3S,20S)-20-formylpregn-5,7,9-trien-3-yl acetate as a colourless solid (1090 mg, 3.08 mmol, 76%).

¹H NMR (400 MHZ, CDCl₃) δ 9.64 (d, J=3.1 Hz, 1H, CHO), 6.90 (d, J=7.9 Hz, 1H, H-6), 6.85 (d, J=7.8 Hz, 1H, H-7), 5.15 (dddd, J=9.1, 7.9, 5.0, 3.1 Hz, 1H, H-3), 3.09 (dd, J=16.3, 5.0 Hz, 1H, H-4), 2.87 (dd, J=16.3, 7.7 Hz, 1H, H-4), 2.77-2.60 (m, 5H, H-14 und CH₂), 2.46 (dqd, J=10.0, 6.8, 3.1 Hz, 1H, H-20), 2.24-2.08 (m, 4H, CH₂), 2.06 (s, 3H, H₃CCOO), 1.98-1.88 (m, 1H, H-2), 1.74 (dt, J=14.5, 8.2 Hz, 2H, H-17, CH₂), 1.60 (ddd, J=10.9, 8.2, 4.1 Hz, 2H, CH₂), 1.21 (d, J=6.8 Hz, 3H, H-21), 0.65 (s, 3H, H-18). ¹³C NMR (101 MHz, CDCl₃) δ 205.0 (CHO), 171.0 (H₃CCOO), 137.3 (C-9), 133.9 (C-8), 133.3 (C-10), 131.2 (C-5), 126.9 (C-6 or C-7), 124.0 (C-6 or C-7), 69.9 (C-3), 51.2 (C-1), 50.2 (C-17), 50.0 (C-20), 42.6 (C-13), 36.8 (C-12), 35.2 (C-4), 28.1 (C-2), 27.9 (CH₂), 25.0 (CH₂), 24.7 (CH₂), 24.3 (CH₂), 21.6 (H₃CCOO), 13.7 (C-21), 11.7 (C-18). HR-MS (EI): calcd. for C₂₃H₃₀O₃: 354.2195, found 294.1978 (—H₃CCOOH).

### (3S,20S)-20-(Hydroxymethyl)-pregn-5,7,9-trien-3-yl Acetate (MiH-45)

(3S,20S)-20-Formylpregn-5,7,9-trien-3-yl acetate (140 mg, 0.395 mmol) was dissolved in a flame dried Schlenk flask under nitrogen atmosphere in a 3:2 mixture of dry CHCl₃/MeOH (4 mL). After the addition of sodium borohydride (15 mg, 0.40 mmol) stirring was continued for 1 hour at room temperature. The reaction was quenched using saturated NH₄Cl solution (3 mL) and the aqueous layer was extracted with CHCl₃ (3×3 mL). The combined organic extracts were dried by filtration through a hydrophobic filter paper and the solvent was removed in vacuo. After purification using FCC (hexane/EtOAc 70:30) the (3S,20S)-20-(hydroxymethyl)-pregn-5,7,9-trien-3-yl acetate was obtained as a colourless solid (130 mg, 0.365 mmol, 92%).

¹H NMR (400 MHZ, CDCl₃) δ 6.87 (q, J=7.9 Hz, 2H, H-6 und H-7), 5.15 (dddd, J=9.1, 8.0, 5.0, 3.1 Hz, 1H, H-3), 3.70 (dd, J=10.5, 3.2 Hz, 1H, H-22), 3.45 (dd, J=10.5, 6.7 Hz, 1H, H-22), 3.09 (dd, J=16.3, 5.1 Hz, 1H, H-4), 2.87 (dd, J=16.3, 7.7 Hz, 1H, H-4), 2.75-2.62 (m, 5H, H-11, H-14, H-15, H16, H-17), 2.25 (ddd, J=13.2, 6.8, 2.5 Hz, 1H, H-12), 2.16-2.08 (m, 3H, H-2, H-15, H-16), 2.06 (s, 3H, H₃CCOO), 1.98-1.88 (m, 1H, H-2), 1.74-1.40 (m, 7H, H-12, H-14, H-15, H-16, H-17, H-20), 1.14 (d, J=6.6 Hz, 3H, H-21), 0.62 (s, 3H, H-18). ¹³C NMR (101 MHz, CDCl₃) δ 171.0 (H₃CCOO), 137.9 (C-9), 134.2 (C-8), 133.2 (C-10), 130.9 (C-5), 126.8 (C-6), 124.0 (C-7), 70.0 (C-3), 68.1 (C-22), 51.6 (C-14 or C-17), 51.6 (C-14 or C-17), 42.1 (C-13), 39.3 (C-20), 36.9 (C-12), 35.2 (C-4), 28.5 (C-1), 28.2 (C-2), 25.2 (C-11), 24.4 (C-15 or C-16), 24.3 (C-15 or C-16), 21.6 (H₃CCOO), 17.0 (C-21), 11.3 (C-18). HR-MS (EI): calcd. for C₂₃H₃₂O₃ 356.2351, found 338.2241 (—H₂O).

### (3S,20S)-20-[(2-Fluoroacryloyloxy)methyl]-pregn-5,7,9-trien-3-yl Acetate (MiH-46, Example 35)

In a flame dried round bottom flask equipped with a stirring bead, (3S,20S)-20-(hydroxymethyl)-pregn-5,7,9-trien-3-yl acetate (36 mg, 0.10 mmol), 2-fluoropropanoic acid (11.2 mg, 0.121 mmol) and DMAP (6.2 mg, 0.051 mmol) were dissolved in dry DCM (2 mL) under nitrogen atmosphere. To the solution was added dropwise a solution of 1-(3-dimethylaminopropyl)-3-ethylcarbodiimide hydrochloride (23.7 mg, 0.121 mmol) in dry DCM (1 mL). The reaction was stirred for 48 hours, then quenched with 1 M HCl (2 mL). The aqueous layer was extracted with DCM (3×3 mL) and the combined organic extracts were washed with water and brine. Before removing the solvent in vacuo, water was removed through filtration through a hydrophobic filter paper. Purification through FCC (hexanes/EtOAc 90:10) afforded (3S,20S)-20-[(2-fluoroacryloyloxy)methyl]-pregn-5,7,9-trien-3-yl acetate as a colourless solid (31.6 mg, 0.0737 mmol, 73%).

¹H NMR (500 MHZ, CDCl₃) δ 6.89 (d, J=7.8 Hz, 1H, H-6), 6.85 (d, J=7.8 Hz, 1H, H-7), 5.68 (dd, J=43.4, 3.2 Hz, 1H, CFCH₂), 5.33 (dd, J=13.0, 3.2 Hz, 1H, CFCH₂), 5.15 (dddd, J=9.2, 7.9, 5.0, 3.0 Hz, 1H, H-3), 4.30 (dd, J=10.8, 3.5 Hz, 1H, H-22), 4.03 (dd, J=10.8, 7.4 Hz, 1H, H-22), 3.09 (dd, J=16.3, 5.0 Hz, 1H, H-4), 2.87 (dd, J=16.2, 7.7 Hz, 1H, H-4), 2.74-2.63 (m, 5H, H-11, H-13), 2.25 (ddd, J=13.0, 7.1, 2.1 Hz, 1H, H-12), 2.17-2.07 (m, 3H, H-1, H-15, H-16), 2.06 (s, 3H, H₃CCOO), 1.98-1.87 (m, 2H, H-2), 1.75-1.67 (m, 1H, H-12), 1.62-1.52 (m, 4H, H-1, H-15, H-16), 1.49-1.42 (m, 1H H-17), 1.14 (d, J=6.6 Hz, 3H, H-21), 0.63 (s, 3H, H-18). ¹³C NMR (126 MHZ, CDCl₃) δ 171.0 (H₃CCOO), 160.8 (d, J=36.3 Hz, OOCCF), 153.6 (d, J=262.3 Hz, OCOCF), 137.6 (C-9), 134.0 (C-8), 133.2 (C-10), 131.0 (C-5), 126.8 (C-6), 124.0 (C-7), 102.7 (d, J=15.3 Hz, CFCH₂), 70.9 (C-22), 69.9 (C-3), 51.9 (C-17), 51.5 (C-14), 42.3 (C-13), 36.9 (C-12), 36.4 (C-20), 35.2 (C-4), 28.5 (C-16), 28.2 (C-2), 25.1 (C-11), 24.4 (C-1 or C-15), 24.3 (C-1 or C-15), 21.6 (H₃CCOO), 17.4 (C-21), 11.3 (C-18). HR-MS (EI): calcd. for C₂₆H₃₃FO₄ 428.2363, found 368.2127 (—H₃CCOOH).

### Example 36

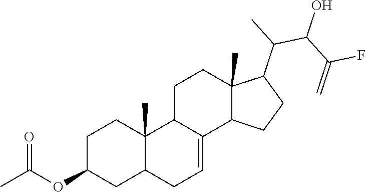 [SMILES: C=C(F)C(O)C(C)C1CCC2C3=CCC4C[C@@H](OC(C)=O)CC[C@]4(C)C3CC[C@@]21C]

### (3S,20RS)-20-(2-Fluoro-1-hydroxyprop-2-enyl)-pregn-7-en-3-yl Acetate (MiH-52)

A solution of tetra-n-butylammonium fluoride (TBAF)(1 M in THF, 0.024 mL, 0.082 mmol) in THF (2 mL) was cooled to −78° C., then fluorovinyldiphenylsilane (0.187 mL, 0.816 mmol) and a solution of (3S,20S)-20-formyl-pregn-7-en-3-yl acetate (152 mg, 0.408 mmol) in dry THF (2 mL) were added dropwise. Stirring was continued for one hour at −78° C., then the mixture was warmed to room temperature and stirred overnight. The reaction then was quenched by the addition of water, the two layers were separated and the aqueous layer was extracted with DCM (3×5 mL). the combined organic layers were dried by filtration through a hydrophobic filter paper and the solvent was removed under reduced pressure. Purification via FCC (hexane/EtOAc 90:10) afforded the (3S,20RS)-20-(2-fluoro-1-hydroxyprop-2-enyl)-pregn-7-en-3-yl acetate as a mixture of epimers as colourless solid (40 mg, 0.096 mmol, 23%).

¹H NMR (500 MHZ, CDCl₃) δ 5.20-5.14 (m, 1H, H-7), 4.76-4.53 (m, 3H, H-3, CFCH₂), 4.48 (s, 1H, H-22), 4.29 (s, 1H, H-22), 2.03 (s, 3H, H₃CCOO), 2.01-1.27 (m, 21H), 1.14 (td, J=14.4, 13.9, 4.0 Hz, 1H, H-4), 0.94 (d, J=6.8 Hz, 2H, H-21), 0.86 (d, J=7.0 Hz, 1H, H-21), 0.81 (s, 3H, H-19), 0.59 (s, 1H, H-18), 0.57 (s, 2H, H-18). ¹³C NMR (126 MHz, CDCl₃) δ 170.9 (H₃CCOO), 166.8 (d, J=259.0 Hz, CF), 166.8 (d, J=258.6 Hz, CF), 139.3 (C-8), 139.1 (C-8), 117.9 (C-7), 117.8 (C-7), 89.8 (d, J=16.9 Hz, CFCH₂), 89.6 (d, J=16.6 Hz, CFCH₂), 73.6 (C-3), 73.6 (C-3), 71.8 (d, J=34.8 Hz, C-22), 70.8 (d, J=34.6 Hz, C-22), 55.1 (C-14), 55.0 (C-14), 52.1 (C-17), 51.7 (C-17), 49.3 (C-5), 43.5 (C-13), 43.3 (C-13), 40.2 (C-12), 39.5 (C-12), 39.2 (C-20), 38.2 (C-20), 37.1 (C-4), 37.0 (C-4), 34.4 (C-10), 33.9 (C-1 or C-2), 29.7 (C-6), 27.7 (C-11), 27.6 (C-11), 27.6 (C-1 or C-2), 23.0 (C-15 or C-16), 22.9 (C-15 or C-16), 21.6 (H₃CCOO), 21.6 (C-15 or C-16), 13.1 (C-19), 13.1 (C-19), 12.3 (C-21), 12.0 (C-18), 12.0 (C-18), 11.8 (C-21). HR-MS (EI): calcd. for C₂₆H₃₉FO₃ 418.2883, found 418.2877.

### Example 37

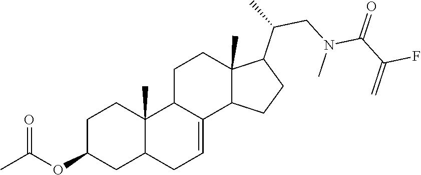 [SMILES: C=C(F)C(=O)N(C)C[C@@H](C)C1CCC2C3=CCC4C[C@@H](OC(C)=O)CC[C@]4(C)C3CC[C@@]21C]

### (3S,20S)-20-[(2-Fluoro-N-methylacrylamido)methyl]-pregn-7-en-3-yl Acetate

In a flame dried round bottom flask equipped with a boiling bead, the secondary amine from example 13 (61 mg, 0.16 mmol), 2-fluoropropanoic acid (17.5 mg, 0.189 mmol) and DMAP (9.6 mg, 0.079 mmol) were dissolved in dry DCM (2 mL) under nitrogen atmosphere. To the solution was added dropwise a solution of 1-(3-dimethylaminopropyl)-3-ethylcarbodiimide hydrochloride (36.9 mg, 0.189 mmol) in dry DCM (1 mL). The reaction was stirred for 48 hours, then quenched with 1 M HCl (2 mL). The aqueous layer was extracted with DCM (3×3 mL) and the combined organic extracts were washed with water (2×5 mL) and brine (5 mL). The combined organic extracts were dried by filtration through a hydrophobic filter paper and the solvent was removed under reduced pressure. Purification through FCC (hexane/EtOAc 70:30) afforded (3S,20S)-20-[(2-fluoro-N-methylacrylamido)methyl]-pregn-7-en-3-yl acetate as a colourless solid (42.3 mg, 0.092 mmol, 58%).

¹H NMR (500 MHZ, CDCl₃; mixture of rotamers) δ 5.31-5.03 (m, 3H, H-7, CFCH₂), 4.69 (tt, J=11.3, 4.6 Hz, 1H, H-3), 3.52-3.08 (m, 2H. H-22), 3.04 (s, 3H, NCH₃), 2.92 (s, 3H, NCH₃), 2.02 (s, 4H, H₃CCOO, with H-12 underneath), 1.97-1.17 (m, 19H), 1.13 (td, J=14.6, 13.9, 3.9 Hz, 1H, H-4), 0.90 (dd, J=13.2, 6.1 Hz, 3H, H-21), 0.81 (s, 3H, H-19), 0.57 (s, 3H, H-18). ¹³C NMR (126 MHZ, CDCl₃; mixture of rotamers) δ 170.8 (H₃CCOO), 163.2 (d, J=29.0 Hz, COCF), 162.8 (d, J=30.1 Hz, COCF), 159.4 (d, J=23.3 Hz, CF), 157.2 (d, J=22.2 Hz, CF), 139.2 (C-8), 139.1 (C-8), 118.0 (C-7), 117.8 (C-7), 99.2 (CFCH₂), 99.1 (CFCH₂), 73.6 (C-3), 55.7 (C-22), 54.8 (C-14), 54.6 (C-14), 53.8 (C-22), 49.3 (C-5), 43.9 (C-13), 40.2 (C-9), 39.5 (C-12), 37.0 (C-4), 36.9 (NCH₃), 35.7 (C-17), 35.2 (C-20), 34.3 (C-10), 34.0 (NCH₃), 33.9 (C-11), 29.6 (C-6), 27.8 (C-1 or C-2), 27.6 (C-1 or C-2), 23.2 (C-16), 21.6 (H₃CCOO), 21.5 (C-15), 16.8 (C-21), 16.3 (C-21), 13.1 (C-19), 12.1 (C-18). HR-MS (EI): calcd. for C₂₈H₄₂FNO₃ 459.3149, found 459.3144.

### Biological Activity of Compounds

### Assay

A GC/MS based screening assay for distal cholesterol biosynthesis inhibitors was used (see Müller et al., Nature Protocols, 2019, 14(8), pp. 2546-2570).

Two test concentrations (1 μM and 50 μM) were used. The compounds were dissolved in ethanol or DMSO and the testing-stock solutions are prepared taking a final dilution of 1:100 into account. Ultimately 10 μL of testing stock solution were added to 990 μL of lipid-free medium containing 1% lipoprotein deficient serum (LPDS) without antibiotics. HL-60 cells (1×10⁶ cells) were incubated in 24-well plates in 1.0 mL of medium consisting of 990 μL of lipid free medium with 1% LPDS and 10 μL of respective inhibitor solution.

After a 24 h incubation period (conditions: 37° C. in a humidified atmosphere containing 5% CO₂) the content of each well was transferred into a 2 mL plastic tube and the wells were washed with 750 μL of phosphate-buffered saline (PBS) and washes are combined with medium samples. The cells were collected by centrifugation at 540×g for 5 min, washed once with 1 mL of cold PBS, and centrifuged again under identical conditions. The samples were transferred to glass vials and one mL of 1 M NaOH is added to each vial and saponification is carried out at 70° C. for 60 min.

After the samples have cooled to room temperature, they were transferred back to the 2 mL plastic vials and lipids are extracted by the addition of 100 μL of internal standard solution (cholestane in MtBE, 10 μg/mL) and 650 μL of MtBE. The tubes were vigorously shaken for 1 min and then centrifuged at 9200×g for 5 min. The extraction was repeated with 750 μL of MtBE and the organic extracts are combined in a plastic tube containing 40 mg of dried sodium sulphate and 5 mg of Bondesil PSA. Samples were subsequently centrifuged for 5 min at 9200×g. One millilitre of the purified extract was transferred into an autosampler glass vial and evaporated to dryness under a mild stream of nitrogen.

To each vial, 950 μL of MtBE and 50 μL of a mixture of N-methyl-N-(trimethylsilyl)trifluoroacetamide (MSTFA) and N-trimethylsilylimidazole (TSIM)(10:1) were added. The silylation reaction was carried out for 1 h at room temperature. The so prepared sample was analyzed on a GC-MS system, equipped with helium as carrier gas and a 5-MS separation column. The obtained sterol patterns will qualitatively reveal the target enzyme in distal cholesterol biosynthesis (for details see Müller et al., Nature Protocols, 2019, 14 (8), pp. 2546-2570).

For IC₅₀ determination, the incorporation of labelled acetate in the target molecule cholesterol is being assessed and the protocol is altered in the following manner: to each well 10 μL of a sterile sodium 2-¹³C-acetate solution (6.25 mg/mL) were added before the addition of the inhibitor solution, leading to a final ¹³C-acetate concentration of 62.5 μg/mL. After saponification 3×25 μL aliquots are taken for protein determination applying the Bradford reagent. After work-up and silylation, GC-MS analysis allows the quantification ¹³C incorporation into cholesterol according to the below given formula. The percentage inhibition (see formula) relative to untreated control samples (0% inhibition) was plotted against the logarithmic inhibitor concentration using Graph Pad Prism 4 (or higher). A bottom level constant equal to 0 is set as constraint using a sigmoidal dose-response model with a various slope. All samples are normalised to their protein. For each concentration the inhibitory percentage is determined in triplicate.

$$\% inhibition = [ 1 - ( \frac{{A}_{S} \times {A}_{I . S . C .} \times P {C}_{C}}{{A}_{C} \times {A}_{I . S . S .} \times P {C}_{S}} ) ] \times 100$$

The above formula is the calculation formula for the percentage inhibition, where A_{S} represents the area sample; A_{I.S.C}. represents the area internal standard control; PC_{C} represents the protein content control; A_{C} represents the area control; A_{I.S.S}. represents the area internal standard sample; and PCs represents the protein content sample.

### Results

The results for are shown in Table 1.

TABLE 1

| Example | DHCR24 | HL60 assay |
|---|---|---|
| No. | Activity | activity [nM] |
| 1 | Y | 3 |
| 2 | Y | — |
| 3 | Y | — |
| 4 | Y | — |
| 5 | Y | 417 |
| 6 | Y | — |
| 7 | Y | — |
| 8 | Y | — |
| 9 | Y | 6 |
| 10 | Y | 203 |
| 11 | Y | 4 |
| 12 | Y | 198 |
| 13 | Y | — |
| 14 | Y | — |
| 15 | Y | — |
| 16 | Y | 805 |
| 17 | Y | — |
| 18 | Y | — |
| 19 | Y | — |
| 20 | Y | — |
| 21 | Y | 16900 |
| 22 | Y | — |
| 23 | Y | — |
| 24 | Y | — |
| 25 | Y | 6 |
| 26 | Y | — |
| 27 | Y | — |
| 28 | Y | 0.1 |
| 29 | Y | 3 |
| 30 | Y | 300 |
| 31 | Y | 280 |
| 32 | Y | — |
| 33 | Y | 175 |
| 34 | Y | 1000 |
| 35 | Y | 130 |
| 36 | Y | — |
| 37 | Y | 110 |

### In Vivo Experiments Using Example 11 (SH42)

Hemizygous APOE*3-Leiden (E3L) mice were crossbred with homozygous human cholesteryl ester transfer protein (CETP) transgenic mice to generate heterozygous E3L.CETP mice on a C57BL/6J background [M. Westerterp et al., “Cholesteryl ester transfer protein decreases high-density lipoprotein and severely aggravates atherosclerosis in APOE*3-Leiden mice”; Arterioscler Thromb Vasc Biol 26, 2552-2559 (2006)]. Because of phenotypical heterogeneity of E3L. CETP mice, non-responder mice were identified by 4-hour fasting plasma lipid levels, i.e., total cholesterol levels <2 mM and triglyceride levels <2 mM, and excluded before special diet treatment. LXRα-deficient mice (also on C57BL/6J background), generated by Deltagen using gene-targeting methods as described [T. Plosch et al., “Abcg5/Abcg8-independent pathways contribute to hepatobiliary cholesterol secretion in mice”; Am J Physiol Gastrointest Liver Physiol, 291, G414-423 (2006)(33)] were provided by Tularik (San Francisco, CA, US). Mice were group-housed in individually ventilated cages in standard conditions at room temperature (22° C.) with 40±5% relative humidity and a 12-h light/dark (7 am lights on; 7 pm lights off) cycle. All animals received humane care according to the criteria outlined in the NIH “Guide for the Care and Use of Laboratory Animals”. All animal procedures were performed in conformance with the guidelines from Directive 2010/63/EU of the European Parliament on the protection of animals used for scientific purposes.

At the age of 10-12 weeks, male E3L.CETP mice and LXRα-deficient mice were fed a high fat high cholesterol diet (HFCD; Altromin, Germany) containing 60% (energy) fat and 1% (wt/wt) cholesterol, and were randomized into two groups treated with either the DHCR24 inhibitor compound of Example 11 [also referred to herein as SH42] (0.5 mg·mouse⁻¹) or vehicle (saline containing 3.3% ethanol and 3.3% Cremophor EL) 3 times per week by intraperitoneal injection. E3L.CETP mice were treated for 4 weeks (n=6 mice per group) and 8 weeks (n=8 mice per group) to evaluate effects on hepatic immune cells via flow cytometry analysis and hepatic steatosis via quantitative lipidomic analysis, respectively. LXRα-deficient mice were treated with either SH42 (n=11 mice) or vehicle (n=10 mice) for 4 weeks to evaluate effects on hepatic steatosis and immune cells. In a rescue experiment, male E3L.CETP mice were fed with HFCD for 10 weeks first and cotreated with SH42 (0.5 mg mouse⁻¹) or vehicle (saline containing 3.3% ethanol and 3.3% Cremophor EL) 3 times per week by intraperitoneal injection for additional 8 weeks.

Body weight was measured weekly. Body composition (i.e. fat body and lean body mass; EchoMRI-100; EchoMRI, Houston, TX, USA) was evaluated before and after intervention. Food intake was determined during the treatment period.

### Results

### Inhibition of DHCR24 by SH42 Markedly Increases Liver Desmosterol Levels and Ameliorates Hepatic Steatosis

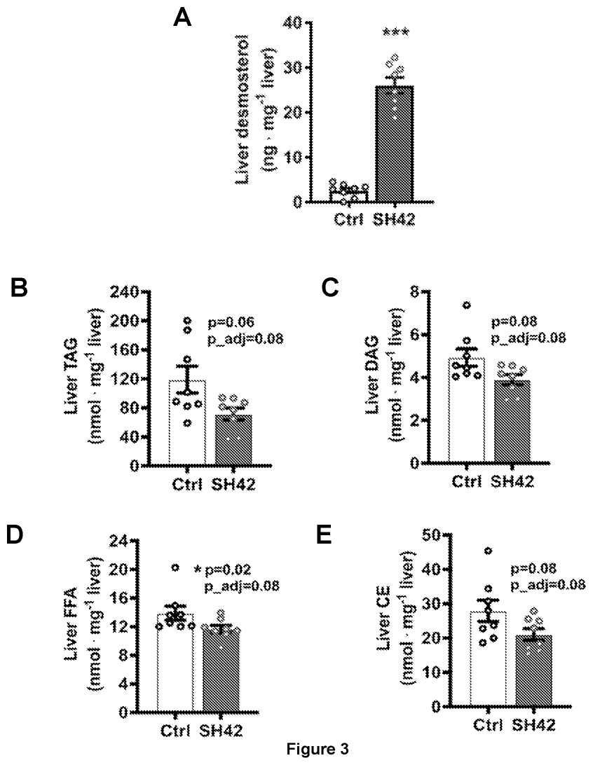

To assess the effects of DHCR24 inhibition on hepatic steatosis, E3L. CETP mice were fed a HFCD while being treated with vehicle or the synthetic DCHR24 inhibitor SH42 for a period of 8 weeks. SH42 treatment did not affect food intake, while temporarily preventing HFCD-induced body weight gain as compared to control group. After 8 weeks of treatment, body weight and body composition, i.e., lean body mass and fat body mass of SH42 treated mice was comparable to that of the control group. In addition, the weight of various tissues (i.e., liver, white adipose tissue, kidney, heart, lung, spleen, and brown adipose tissue) was unchanged by SH42 treatment. SH42 markedly increased hepatic desmosterol levels (10-fold, see FIG. 3A).

Hepatic steatosis was evaluated by HE staining and scored. As compared to the control treatment, SH42 treatment ameliorated diet-induced hepatic steatosis, as was evident by a clear reduction of the hepatic steatosis score (−58%), and liver lipid area (−35%). Liver lipid profiles were analysed by comprehensive lipidomic analysis. Firstly, a clear alteration in the lipid class composition after SH42 treatment was observed. Specifically, SH42 treatment caused a relative reduction of TAG (−21%) and DAG (−22%), accompanied by a relative increase of the other lipid classes, including CER, PC, PE, and SM.

The majority of significantly altered lipid species were down-regulated, and most of them were TAGs. Consistently, SH42 tended to decrease hepatic concentrations of triacylglycerides (TAG)(−39%; see FIG. 3B) and diacylglycerides (DAG)(−20%; see FIG. 3C). In addition, SH42 treatment reduced hepatic free fatty acids (FFA)(−16%; see FIG. 3D) and cholesterol esterase (CE) levels (−25%; see FIG. 3E). Despite these strong effects on hepatic lipid content, SH42 did not affect fasting plasma glucose, insulin, or homeostatic Model Assessment for Insulin Resistance (HOMA-IR) scores. Taken together, inhibition of DHCR24 by SH42 markedly increases liver desmosterol levels, accompanied by amelioration of diet-induced hepatic steatosis without marked effects on body composition and glucose homeostasis.

Inhibition of DHCR24 by SH42 Prevents Kupffer Cell Activation and Reduces Immune Cell Infiltration into the Liver

The effect of DHCR24 inhibition on the hepatic inflammation in mice treated with SH42 or vehicle for 8 weeks by immunohistochemistry. SH42 treatment significantly reduced the hepatic F4/80 content (−29%) as well as the number of hepatic crown-like structures of macrophages surrounding dying hepatocytes (−79%), which is a prominent feature of NASH development.

Flow cytometry on MACS-purified hepatic leukocytes revealed that 4 weeks SH42 treatment tended to decrease total hepatic leukocytes (−37%; p=0.09). A significant increase in total Kupffer cells (KCs) were observed upon SH42 treatment (+44%). Although, SH42 did not significantly reduce MHCII⁺/CD11c⁺ activated KCs, MHCII⁻/CD11c⁻ resting KCs were significantly increased (+21%), indicative of prevented KC activation. In addition, SH42 treatment reduced monocytes in both liver (−79%) and blood (−43%; p=0.06), and decreased hepatic neutrophils (−50%) without affecting circulating neutrophils. Together, these data indicate that SH42 treatment prevents KC activation, limits hepatic immune cell recruitment and dampens hepatic inflammation.

Inhibition of DHCR24 by SH42 does not Increase Circulating Lipids

Consistent with the potent increase in hepatic desmosterol levels, SH42 also markedly increased plasma desmosterol levels from undetectable levels (<0.5 μg·mL⁻¹) to 3.1±0.4 μg·mL⁻¹. Since synthetic LXR agonists usually induce lipogenesis and hypertriglyceridemia as unwanted effects, the effect of 8 weeks of SH42 treatment on circulating lipid levels was determined using quantitative comprehensive lipidomic analysis. Analysis of the plasma lipidome revealed that SH42 treatment relatively decreased circulating CE while relatively increasing lactosylceramides (LCER), phosphatidylcholine (PC) and phosphatidylethanolamine (PE). With respect to absolute lipid concentrations, SH42 did not affect plasma levels of total TAG and DAG, while significantly decreasing plasma levels of FFA (−16%) and CE (−24%; p=0.08). These data imply that inhibition of DHCR24 by SH42 increases plasma desmosterol levels and decreases FFA and CE levels, importantly without inducing hypertriglyceridemia.

### Inhibition of DHCR24 by SH42 Delays High Fat Diet-Induced Liver Inflammation, Fibrosis and Injury

E3L. CETP mice were fed with a HFCD for 10 weeks first to establish NAFLD, and then treated with vehicle or SH42 while still on HFCD for additional 8 weeks. SH42 treatment did not significantly influence either total body weight or composition. No difference in liver weight and hepatic steatosis was observed, SH42 treatment reduced liver inflammation mainly by reducing hepatic crown-like structures (−89%) without significant influence on the F4/80 positive area, and ameliorated liver fibrosis as evidenced by reduced collagen content (−50%). In addition, SH42 treatment reduced plasma levels of the liver injury marker, plasma alanine transaminase (ALT)(−42%). These effects were accompanied with a robust increase in plasma desmosterol levels (>400-fold). In summary, inhibition of DHCR24 by SH42 delays high fat diet-induced NAFLD/NASH progression from simple steatosis to advanced stages with severe liver inflammation, fibrosis and injury.

### In Vivo Experiments Using Example 11 (SH42) and Example 33 (MiH-17)

Experiments were performed to determine if the compound of Example 33 (MIH17) increased brain and circulating desmosterol levels.

Mice received a daily intraperitoneally injection with either vehicle (saline containing 6% ethanol and 6% Cremophor EL; 5 μl/g mouse) and either SH42 or MIH17 (50 μg/g mouse; 5 μl/g mouse) for three days. 24 hours after the last injection and after 4 hours of fasting (10-14 h), the mice were killed by CO₂ inhalation (in accordance with Zenya: ‘Euthanasia with the use of the CO₂ box (versie 4)’) after which blood was collected via heart puncture and organs were isolated. Desmosterol levels were determined by GC/MS as described below.

To each sample, 5 μL of an internal standard mixture consisting of 1 mg/mL cholesterol, 20 μg/mL desmosterol-d6 and 20 μg/mL 25-OH-cholesterol-d6 was added. Lipids were extracted twice into methyl-tert-butyl ether after hydrolysis in 80% (v/v) ethanolic sodium hydroxide (1 M). After drying under nitrogen and derivatization with N-methyl-N-(trimethylsilyl)trifluoroacetamide, sterols were analysed on a Agilent GC-MS 5977B gas chromatograph coupled to a single quadrupole mass analyzer. For sterol quantification the following ion traces were monitored: 333 and 327 for respectively desmosterol-d6 and desmosterol. Group comparison was done based on area ratios, correcting the desmosterol signal for its internal standard desmosterol d6.

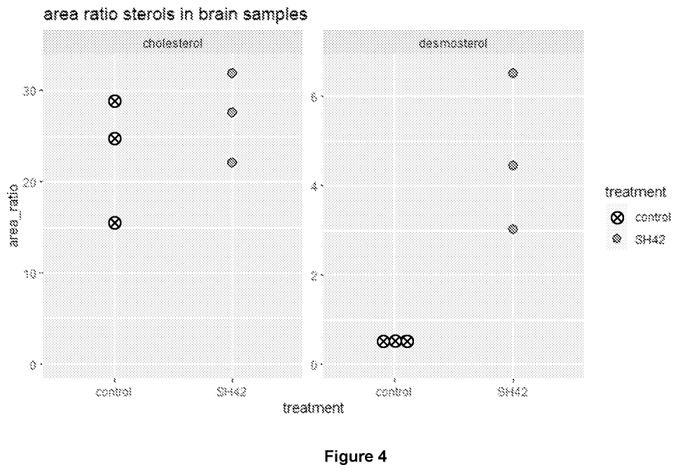

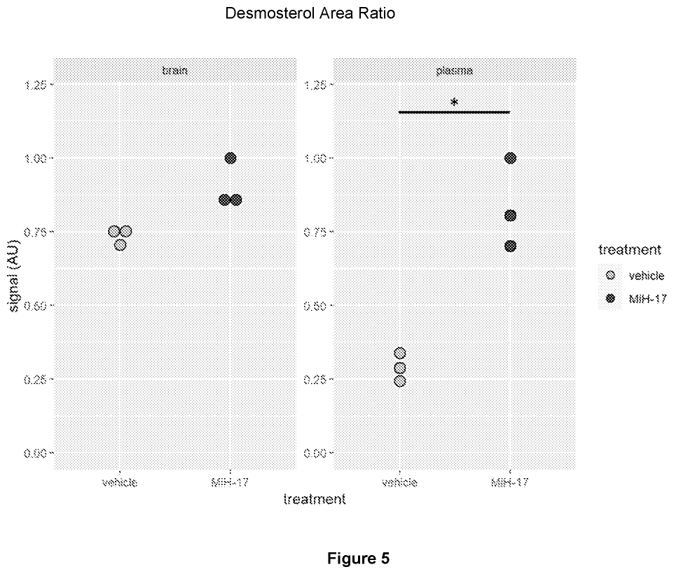

The desmosterol signals that were detected in the samples are shown in FIG. 4 and FIG. 5 (FIG. 5; brain: p-value 0.059; plasma: p-value 0.017).

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g. “such as”) provided herein, is intended merely to better illuminate the invention and does not pose a limitation on the scope of the invention unless otherwise paragraphed. No language in the specification should be construed as indicating any non-paragraphed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the paragraphs appended hereto as permitted by applicable law.

1.-27. (canceled)

28. A method of treating or preventing a DHCR24-mediated disorder, wherein the method comprises administering to a subject a compound of formula (10) or a pharmaceutically acceptable salt, solvate, hydrate or prodrug thereof, wherein the DHCR24-mediated disorder is selected from metabolic dysfunction-associated steatohepatitis (MASH) a.k.a. non-alcoholic steatohepatitis (NASH), atherosclerotic cardiovascular disease (asCVD), multiple sclerosis, hepatocellular carcinoma, breast cancer, endometrial carcinoma, B-cell lymphoma, drug-resistant cancer, hepatitis C, liver inflammation, liver fibrosis or liver injury, wherein the compound of formula (10) is: 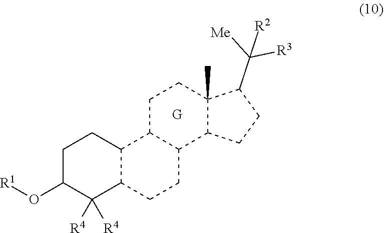
wherein:
G is a fused ring system selected from: 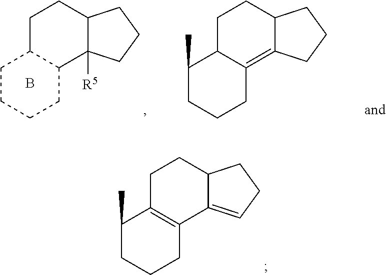
B is selected from: 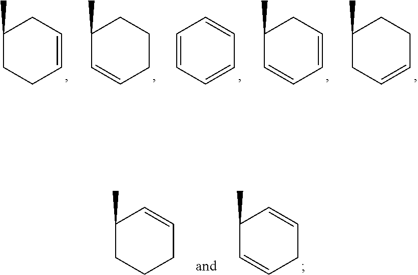
R¹ is selected from hydrogen and —C(═O)R⁶;
R² is selected from hydrogen and C₁₋₆ alkyl;
R³ is selected from a group represented by formula (2), —[C(R⁷)₂]ₙ—X, —CH═CR⁶R⁷, —CR⁷═N—N(R⁶)₂, 5- to 10-membered heteroaryl and 5- to 10-membered heterocycloalkyl, wherein the 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy; 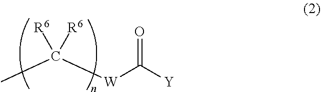
n is an integer from 1 to 6;
W is selected from O and NR⁶;
Y is selected from hydrogen and C₂ alkenyl, wherein the C₂ alkenyl is optionally substituted with one or more halo;
X is selected from halo, —OH, —SH, —O—Z, —S—Z, —S—S—Z, 5- to 10-membered heteroaryl and 5- to 10-membered heterocycloalkyl, wherein the 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy;
each R⁴ is independently selected from hydrogen and C₁₋₆ alkyl;
R⁵ is selected from hydrogen and C₁₋₆ alkyl;
each R⁶ is independently selected from hydrogen and C₁₋₆ alkyl;
each R⁷ is independently selected from H, C₁₋₆ alkyl and C₂₋₆ alkenyl, wherein the C₁₋₆ alkyl and C₂₋₆ alkenyl are optionally substituted with one or more halo;
Z is selected from C₆₋₁₀ aryl, 5- to 10-membered heteroaryl and 5- to 10-membered heterocycloalkyl, wherein the C₆₋₁₀ aryl, 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy; and
wherein the prodrug is an ester, an amide, a hydrazone or a disulfide of the compound of formula (10).

29. The method according to claim 28, wherein the treatment or prevention of metabolic dysfunction-associated steatohepatitis (MASH) a.k.a. non-alcoholic steatohepatitis (NASH) or atherosclerotic cardiovascular disease (asCVD) is without inducing at least one of hyperlipidemia or hypertriglyceridemia.

30. The method according to claim 28, wherein G is: 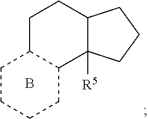
and B is selected from:

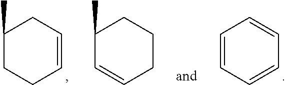

31. The method according to claim 28, wherein R³ is a group represented by formula (2): 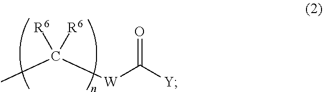
wherein each R⁶ is independently selected from hydrogen, methyl and ethyl.

32. The method according to claim 28, wherein R³ is —CR⁷═N—N(R⁶)₂.

33. The method according to claim 28, wherein R³ is selected from 5- to 10-membered heteroaryl and 5- to 10-membered heterocycloalkyl, wherein the 5- to 10-membered heteroaryl and 5- to 10-membered heterocycloalkyl each contain at least one nitrogen atom.

34. The method according to claim 28, wherein R⁶ is H.

35. The method according to claim 28, wherein R₇ is selected from hydrogen, C₁₋₆ alkyl, halo-C₁₋₆ alkyl, C₂₋₆ alkenyl and halo-C₂₋₆ alkenyl.

36. The method according to claim 28, wherein the compound of formula (10) is:

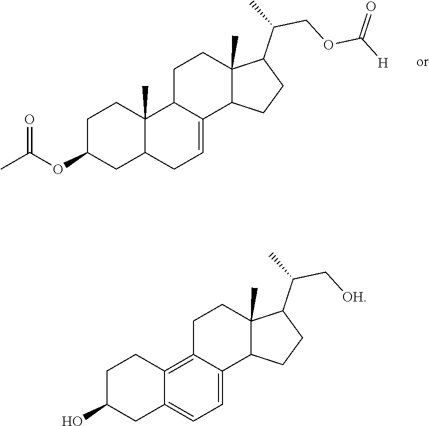 [SMILES: CC(=O)O[C@H]1CC[C@@]2(C)C(CC=C3C2CC[C@@]2(C)C3CCC2[C@H](C)COC=O)C1 ; "or" ; C[C@H](CO)C1CCC2c3ccc4c(c3CC[C@@]21C)CC[C@H](O)C4]

37. The method according to claim 28, wherein the compound of formula (10) is selected from:

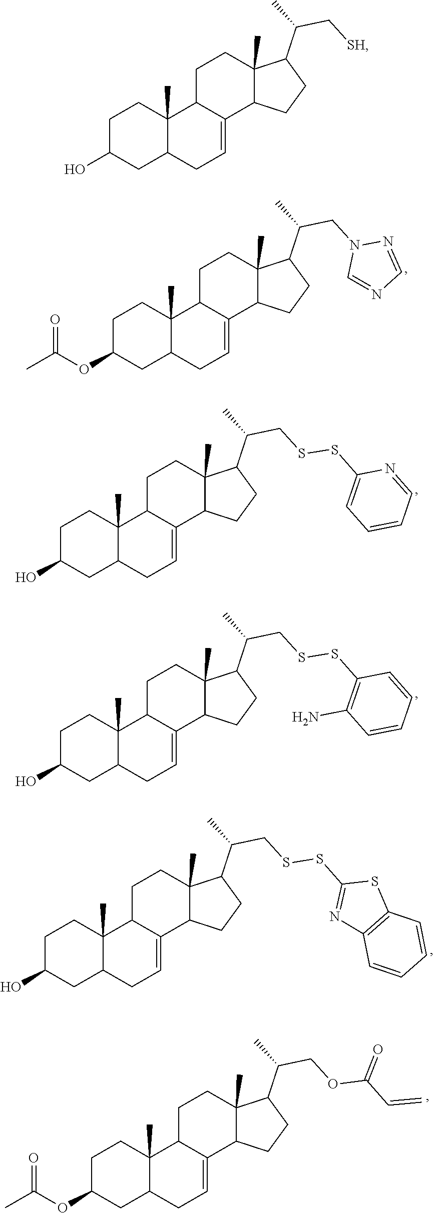 [SMILES: C[C@H](CS)C1CCC2C3=CCC4CC(O)CC[C@]4(C)C3CC[C@@]21C ; CC(=O)O[C@H]1CC[C@@]2(C)C(CC=C3C2CC[C@@]2(C)C3CCC2[C@H](C)Cn2cncn2)C1 ; C[C@H](CSSc1ccccn1)C1CCC2C3=CCC4C[C@@H](O)CC[C@]4(C)C3CC[C@@]21C ; C[C@H](CSSc1ccccc1N)C1CCC2C3=CCC4C[C@@H](O)CC[C@]4(C)C3CC[C@@]21C ; C[C@H](CSSc1nc2ccccc2s1)C1CCC2C3=CCC4C[C@@H](O)CC[C@]4(C)C3CC[C@@]21C ; C=CC(=O)OC[C@@H](C)C1CCC2C3=CCC4C[C@@H](OC(C)=O)CC[C@]4(C)C3CC[C@@]21C]

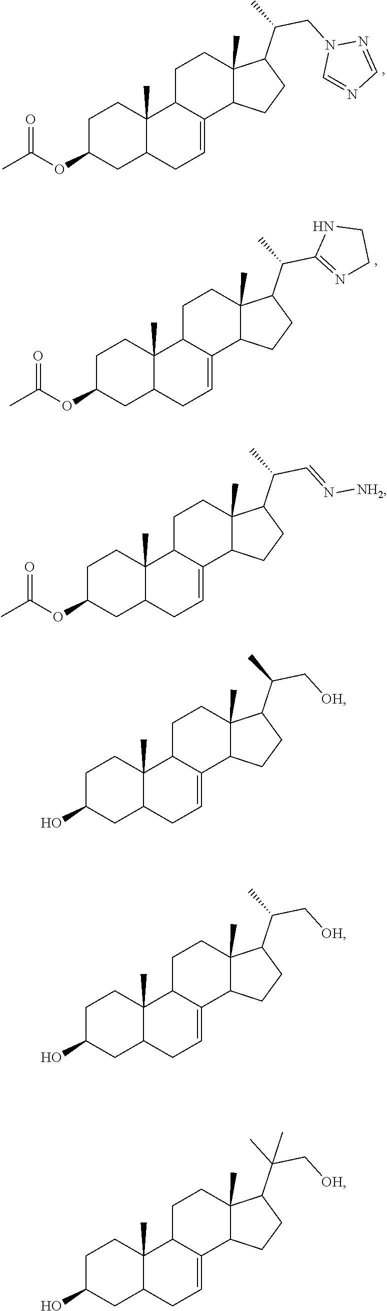 [SMILES: CC(=O)O[C@H]1CC[C@@]2(C)C(CC=C3C2CC[C@@]2(C)C3CCC2[C@H](C)Cn2cncn2)C1 ; CC(=O)O[C@H]1CC[C@@]2(C)C(CC=C3C2CC[C@@]2(C)C3CCC2[C@H](C)C2=NCCN2)C1 ; CC(=O)O[C@H]1CC[C@@]2(C)C(CC=C3C2CC[C@@]2(C)C3CCC2[C@H](C)/C=N/N)C1 ; C[C@@H](CO)C1CCC2C3=CCC4C[C@@H](O)CC[C@]4(C)C3CC[C@@]21C ; C[C@H](CO)C1CCC2C3=CCC4C[C@@H](O)CC[C@]4(C)C3CC[C@@]21C ; CC(C)(CO)C1CCC2C3=CCC4C[C@@H](O)CC[C@]4(C)C3CC[C@@]21C]

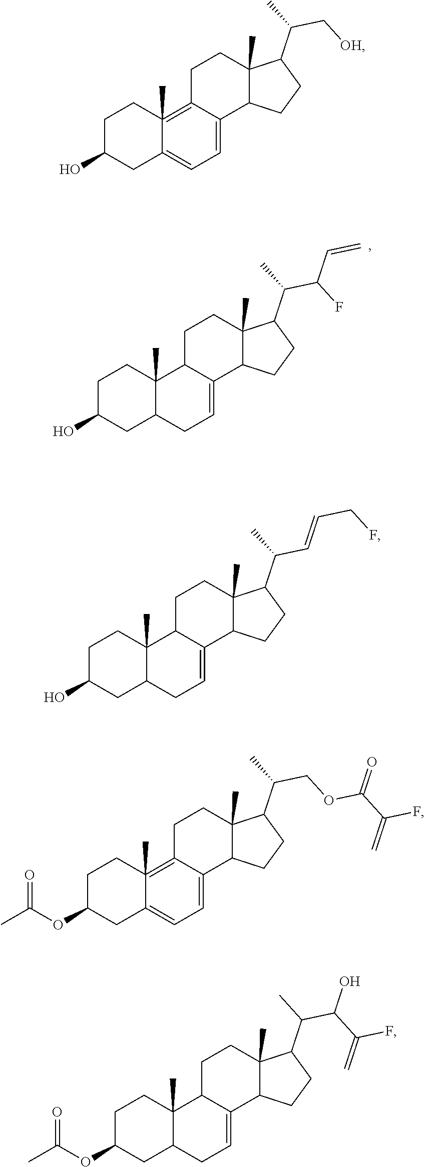

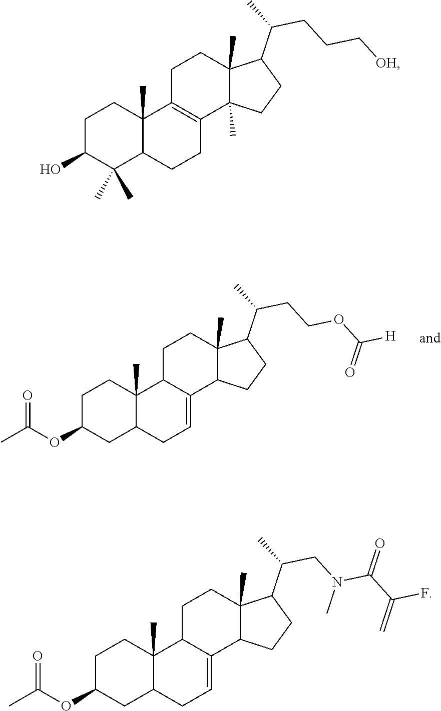

38. A compound of formula (10) or a salt, solvate, hydrate or prodrug thereof, 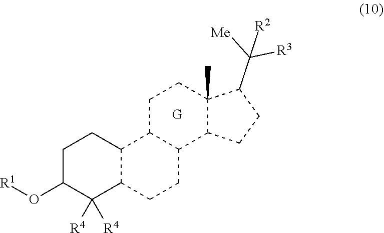
wherein:
G is a fused ring system selected from: 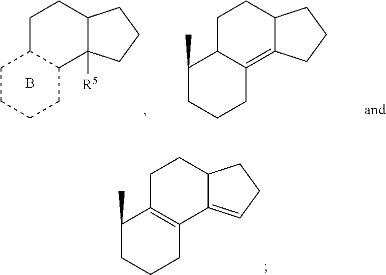
B is selected from: 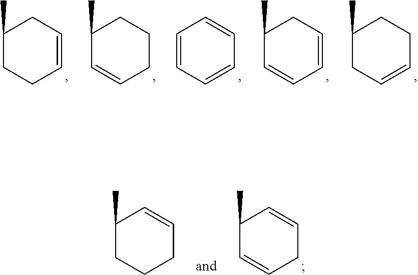
R¹ is selected from hydrogen and —C(═O)R⁶;
R² is selected from hydrogen and C₁₋₆ alkyl;
R³ is selected from a group represented by formula (2), —[C(R⁷)₂]ₙ—X, —CH═CR⁶R⁷, —CR⁷═N—N(R⁶)₂, 5- to 10-membered heteroaryl and 5- to 10-membered heterocycloalkyl, wherein the 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy; 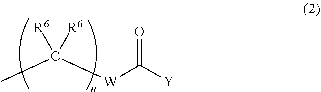
n is an integer from 1 to 6;
W is selected from O and NR⁶;
Y is selected from hydrogen and C₂ alkenyl, wherein the C₂ alkenyl is optionally substituted with one or more halo;
X is selected from halo, —OH, —SH, —O—Z, —S—Z, —S—S—Z, 5- to 10-membered heteroaryl and 5- to 10-membered heterocycloalkyl, wherein the 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy;
each R⁴ is independently selected from hydrogen and C₁₋₆ alkyl;
R⁵ is selected from hydrogen and C₁₋₆ alkyl;
each R⁶ is independently selected from hydrogen and C₁₋₆ alkyl;
each R⁷ is independently selected from H, C₁₋₆ alkyl and C₂₋₆ alkenyl, wherein the C₁₋₆ alkyl and C₂₋₆ alkenyl are optionally substituted with one or more halo;
Z is selected from C₆₋₁₀ aryl, 5- to 10-membered heteroaryl and 5- to 10-membered heterocycloalkyl, wherein the C₆₋₁₀ aryl, 5- to 10-membered heteroaryl and 5- to 10-membered heterocycloalkyl are optionally substituted by one or more groups selected from —OH, halo, —CN, —NH₂, —NO₂, C₁₋₆ alkyl and C₁₋₆ alkoxy; and
wherein the prodrug is an ester, an amide, a hydrazone or a disulfide of the compound of formula (10); and
wherein the compound is not one of:

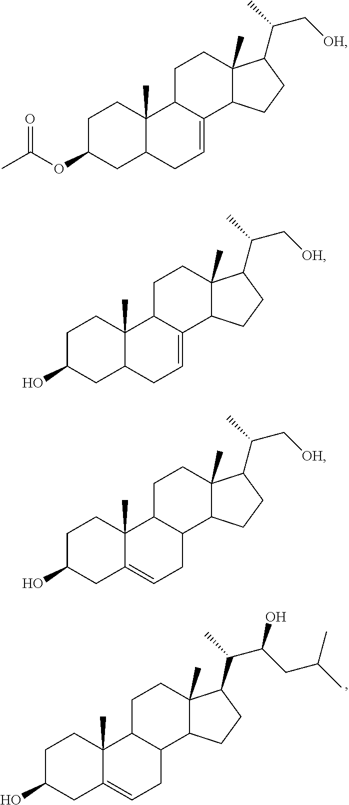 [SMILES: CC(=O)O[C@H]1CC[C@@]2(C)C(CC=C3C2CC[C@@]2(C)C3CCC2[C@H](C)CO)C1 ; C[C@H](CO)C1CCC2C3=CCC4C[C@@H](O)CC[C@]4(C)C3CC[C@@]21C ; C[C@H](CO)C1CCC2C3CC=C4C[C@@H](O)CC[C@]4(C)C3CC[C@@]21C ; CC(C)C[C@H](O)[C@@H](C)[C@H]1CCC2C3CC=C4C[C@@H](O)CC[C@]4(C)C3CC[C@@]21C]

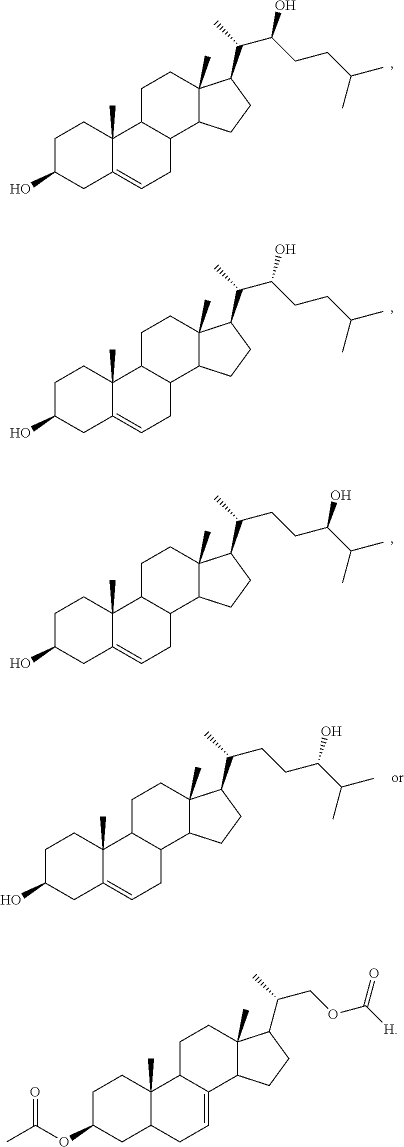

39. The compound according to claim 38, wherein G is: 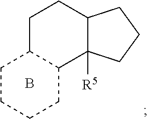
and B is selected from:

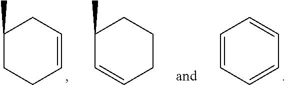

40. The compound according to claim 38, wherein R³ is a group represented by formula (2): 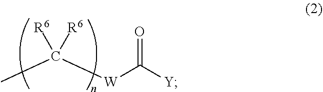
wherein each R⁶ is independently selected from hydrogen, methyl and ethyl.

41. The compound according to claim 38, wherein R³ is —CR⁷═N—N(R⁶)₂.

42. The compound according to claim 38, wherein R³ is selected from 5- to 10-membered heteroaryl and 5- to 10-membered heterocycloalkyl, wherein the 5- to 10-membered heteroaryl and 5- to 10-membered heterocycloalkyl each contain at least one nitrogen atom.

43. The compound according to claim 38, wherein R⁶ is H.

44. The compound according to claim 38, wherein R₇ is selected from hydrogen, C₁₋₆ alkyl, halo-C₁₋₆ alkyl, C₂₋₆ alkenyl and halo-C₂₋₆ alkenyl.

45. The compound according to claim 38, which is:

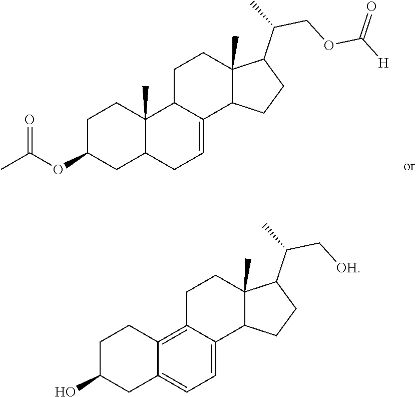 [SMILES: CC(=O)O[C@H]1CC[C@@]2(C)C(CC=C3C2CC[C@@]2(C)C3CCC2[C@H](C)COC=O)C1 ; C[C@H](CO)C1CCC2c3ccc4c(c3CC[C@@]21C)CC[C@H](O)C4]

46. The compound according to claim 38, which is selected from: 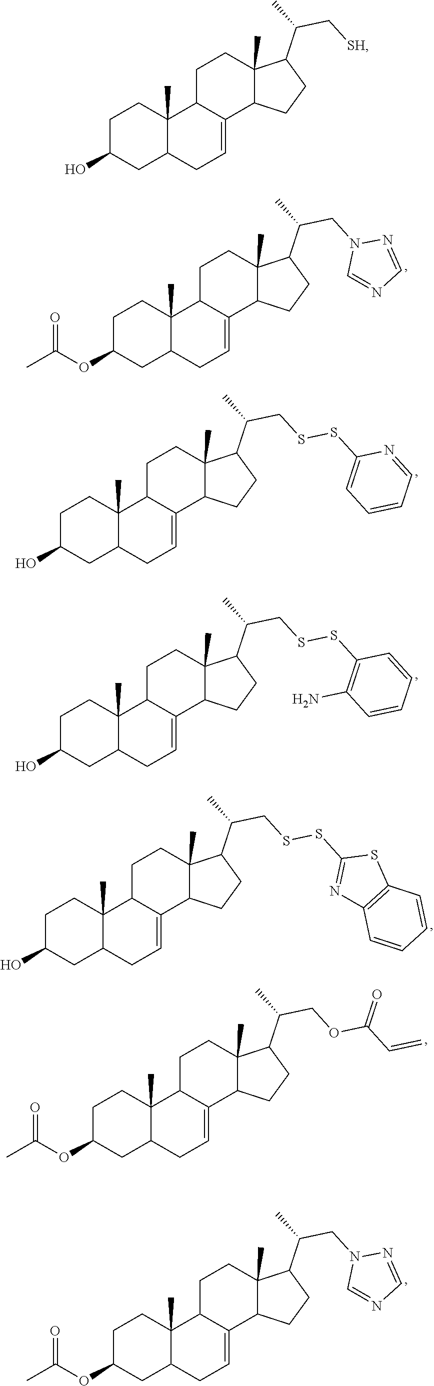 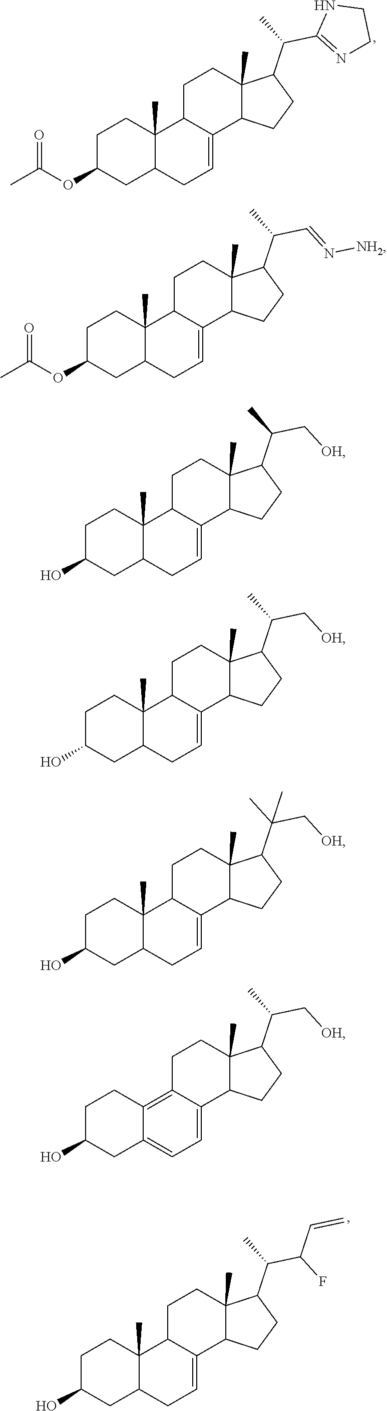 [chemical structure]
